SEQUENCE LISTING (80 sequences)

SEQ ID NO: 1 - protein, 14 aa, Artificial sequence
  Synthetic peptide
IEGPTLRQWLAARA

SEQ ID NO: 2 - protein, 28 aa, Artificial sequence
  Synthetic peptide
IEGPTLRQWLAARAARAALWQRLTPGEI

SEQ ID NO: 3 - DNA, 684 bp, Homo sapiens
atggacaaaactcacacatgtccaccttgtccagctccggaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 4 - DNA, 684 bp, Homo sapiens
tacctgttttgagtgtgtacaggtggaacaggtcgaggccttgaggacccccctggcagtcagaaggagaaggggggttttgggttcctgtgggagtactagagggcctggggactccagtgtacgcaccaccacctgcactcggtgcttctgggactccagttcaagttgaccatgcacctgccgcacctccacgtattacggttctgtttcggcgccctcctcgtcatgttgtcgtgcatggcacaccagtcgcaggagtggcaggacgtggtcctgaccgacttaccgttcctcatgttcacgttccagaggttgtttcgggagggtcgggggtagctcttttggtagaggtttcggtttcccgtcggggctcttggtgtccacatgtgggacgggggtagggccctactcgactggttcttggtccagtcggactggacggaccagtttccgaagatagggtcgctgtagcggcacctcaccctctcgttacccgtcggcctcttgttgatgttctggtgcggagggcacgacctgaggctgccgaggaagaaggagatgtcgttcgagtggcacctgttctcgtccaccgtcgtccccttgcagaagagtacgaggcactacgtactccgagacgtgttggtgatgtgcgtcttctcggagagggacagaggcccattt

SEQ ID NO: 5 - protein, 228 aa, Artificial sequence
  Synthetic peptide
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 8 aa, Artificial sequence
  Synthetic peptide
GGGKGGGG

SEQ ID NO: 7 - protein, 8 aa, Artificial sequence
  Synthetic peptide
GGGNGSGG

SEQ ID NO: 8 - protein, 8 aa, Artificial sequence
  Synthetic peptide
GGGCGGGG

SEQ ID NO: 9 - protein, 4 aa, Artificial sequence
  Synthetic peptide
GPNG

SEQ ID NO: 10 - protein, 32 aa, Artificial sequence
  Synthetic peptide
IEGPTLRQWLAARAGPNGIEGPTLRQWLAARA

SEQ ID NO: 11 - protein, 36 aa, Artificial sequence
  Synthetic peptide
IEGPTLRQCLAARAGGGGGGGGIEGPTLRQCLAARA

SEQ ID NO: 12 - protein, 36 aa, Artificial sequence
  Synthetic peptide
IEGPTLRQCLAARAGGGGGGGGIEGPTRLQCLAARA

SEQ ID NO: 13 - protein, 36 aa, Artificial sequence
  Synthetic peptide
IEGPTLRQALAARAGGGGGGGGIEGPTLRQALAARA

SEQ ID NO: 14 - protein, 36 aa, Artificial sequence
  Synthetic peptide
IEGPTLRQWLAARAGGGKGGGGIEGPTLRQWLAARA

SEQ ID NO: 15 - protein, 36 aa, Artificial sequence; identical to SEQ ID NO: 14
  Synthetic peptide

SEQ ID NO: 16 - protein, 36 aa, Artificial sequence
  Synthetic peptide
IEGPTLRQWLAARAGGGCGGGGIEGPTLRQWLAARA

SEQ ID NO: 17 - protein, 36 aa, Artificial sequence; identical to SEQ ID NO: 14
  Synthetic peptide

SEQ ID NO: 18 - protein, 36 aa, Artificial sequence; identical to SEQ ID NO: 16
  Synthetic peptide

SEQ ID NO: 19 - protein, 36 aa, Artificial sequence
  Synthetic peptide
IEGPTLRQWLAARAGGGNGSGGIEGPTLRQWLAARA

SEQ ID NO: 20 - protein, 36 aa, Artificial sequence; identical to SEQ ID NO: 16
  Synthetic peptide

SEQ ID NO: 21 - protein, 36 aa, Artificial sequence
  Synthetic peptide
IEGPTLRQWLAARAGGGGGGGGIEGPTLRQWLAARA

SEQ ID NO: 22 - protein, 32 aa, Artificial sequence; identical to SEQ ID NO: 10
  Synthetic peptide

SEQ ID NO: 23 - protein, 32 aa, Artificial sequence; identical to SEQ ID NO: 10
  Synthetic peptide

SEQ ID NO: 24 - protein, 36 aa, Artificial sequence; identical to SEQ ID NO: 21
  Synthetic peptide

SEQ ID NO: 25 - protein, 34 aa, Artificial sequence
  Synthetic peptide
GGIEGPTLRQWLAARAGPNGIEGPTLRQWLAARA

SEQ ID NO: 26 - protein, 36 aa, Artificial sequence; identical to SEQ ID NO: 21
  Synthetic peptide

SEQ ID NO: 27 - protein, 36 aa, Artificial sequence; identical to SEQ ID NO: 11
  Synthetic peptide

SEQ ID NO: 28 - protein, 36 aa, Artificial sequence; identical to SEQ ID NO: 11
  Synthetic peptide

SEQ ID NO: 29 - protein, 36 aa, Artificial sequence; identical to SEQ ID NO: 13
  Synthetic peptide

SEQ ID NO: 30 - protein, 36 aa, Artificial sequence; identical to SEQ ID NO: 14
  Synthetic peptide

SEQ ID NO: 31 - protein, 36 aa, Artificial sequence; identical to SEQ ID NO: 16
  Synthetic peptide

SEQ ID NO: 32 - protein, 36 aa, Artificial sequence; identical to SEQ ID NO: 19
  Synthetic peptide

SEQ ID NO: 33 - protein, 36 aa, Artificial sequence; identical to SEQ ID NO: 16
  Synthetic peptide

SEQ ID NO: 34 - protein, 41 aa, Artificial sequence
  Synthetic peptide
GGGGGIEGPTLRQWLAARAGGGGGGGGIEGPTLRQWLAARA

SEQ ID NO: 35 - DNA, 60 bp, Artificial sequence
  Synthetic polynucleotide
aaaggtggaggtggtggtatcgaaggtccgactctgcgtcagtggctggctgctcgtgct

SEQ ID NO: 36 - DNA, 48 bp, Artificial sequence
  Synthetic polynucleotide
acctccaccaccagcacgagcagccagccactgacgcagagtcggacc

SEQ ID NO: 37 - DNA, 66 bp, Artificial sequence
  Synthetic polynucleotide
ggtggtggaggtggcggcggaggtattgagggcccaacccttcgccaatggcttgcagcacgcgca

SEQ ID NO: 38 - DNA, 76 bp, Artificial sequence
  Synthetic polynucleotide
aaaaaaaggatcctcgagattatgcgcgtgctgcaagccattggcgaagggttgggccctcaatacctccgccgcc

SEQ ID NO: 39 - DNA, 126 bp, Artificial sequence
  Synthetic polynucleotide
aaaggtggaggtggtggtatcgaaggtccgactctgcgtcagtggctggctgctcgtgctggtggtggaggtggcggcggaggtattgagggcccaacccttcgccaatggcttgcagcacgcgca

SEQ ID NO: 40 - DNA, 124 bp, Artificial sequence
  Synthetic polynucleotide
ccaggctgagacgcagtcaccgaccgacgagcacgaccaccacctccaccgccgcctccataactcccgggttgggaagcggttaccgaacgtcgtgcgcgtattagagctcctaggaaaaaaa

SEQ ID NO: 41 - protein, 42 aa, Artificial sequence
  Synthetic peptide
KGGGGGIEGPTLRQWLAARAGGGGGGGGIEGPTLRQWLAARA

SEQ ID NO: 42 - DNA, 22 bp, Artificial sequence
  Synthetic polynucleotide
aacataagtacctgtaggatcg

SEQ ID NO: 43 - DNA, 52 bp, Artificial sequence
  Synthetic polynucleotide
ttcgataccaccacctccacctttacccggagacagggagaggctcttctgc

SEQ ID NO: 44 - DNA, 861 bp, Homo sapiens
tctagatttgttttaactaattaaaggaggaataacatatggacaaaactcacacatgtccaccttgtccagctccggaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggaggtggtggtatcgaaggtccgactctgcgtcagtggctggctgctcgtgctggtggtggaggtggcggcggaggtattgagggcccaacccttcgccaatggcttgcagcacgcgcataatctcgaggatccg

SEQ ID NO: 45 - DNA, 861 bp, Homo sapiens
agatctaaacaaaattgattaatttcctccttattgtatacctgttttgagtgtgtacaggtggaacaggtcgaggccttgaggacccccctggcagtcagaaggagaaggggggttttgggttcctgtgggagtactagagggcctggggactccagtgtacgcaccaccacctgcactcggtgcttctgggactccagttcaagttgaccatgcacctgccgcacctccacgtattacggttctgtttcggcgccctcctcgtcatgttgtcgtgcatggcacaccagtcgcaggagtggcaggacgtggtcctgaccgacttaccgttcctcatgttcacgttccagaggttgtttcgggagggtcgggggtagctcttttggtagaggtttcggtttcccgtcggggctcttggtgtccacatgtgggacgggggtagggccctactcgactggttcttggtccagtcggactggacggaccagtttccgaagatagggtcgctgtagcggcacctcaccctctcgttacccgtcggcctcttgttgatgttctggtgcggagggcacgacctgaggctgccgaggaagaaggagatgtcgttcgagtggcacctgttctcgtccaccgtcgtccccttgcagaagagtacgaggcactacgtactccgagacgtgttggtgatgtgcgtcttctcggagagggacagaggcccatttccacctccaccaccatagcttccaggctgagacgcagtcaccgaccgacgagcacgaccaccacctccaccgccgcctccataactcccgggttgggaagcggttaccgaacgtcgtgcgcgtattagagctcctaggc

SEQ ID NO: 46 - protein, 269 aa, Artificial sequence [sequence omitted]

SEQ ID NO: 47 - protein, 14 aa, Artificial sequence
  Synthetic peptide
IEGPTLRQALAARA

SEQ ID NO: 48 - protein, 31 aa, Artificial sequence
  Synthetic peptide
IEGPTLRQALAARAXKXARAALAQRLTPGEI

SEQ ID NO: 49 - protein, 31 aa, Artificial sequence; identical to SEQ ID NO: 48
  Synthetic peptide

SEQ ID NO: 50 - protein, 31 aa, Artificial sequence; identical to SEQ ID NO: 48
  Synthetic peptide

SEQ ID NO: 51 - protein, 29 aa, Artificial sequence
  Synthetic peptide
IEGPTLRQALAARXKXRAALAQRLTPGEI

SEQ ID NO: 52 - protein, 30 aa, Artificial sequence
  Synthetic peptide
IEGPTLRQWLAARAKXARAALWQRLTPGEI

SEQ ID NO: 53 - protein, 30 aa, Artificial sequence
  Synthetic peptide
IEXPTLRQALAARAKXARAALAQRLTPXEI

SEQ ID NO: 54 - protein, 29 aa, Artificial sequence
  Synthetic peptide
IEGFTLRQWLAARAKARAALWQRLTFGEI

SEQ ID NO: 55 - protein, 31 aa, Artificial sequence
  Synthetic peptide
IEGFTLRQWLAARAXKXARAALWQRLTFGEI

SEQ ID NO: 56 - protein, 29 aa, Artificial sequence
  Synthetic peptide
IEGFTLRQWLAARXKXRAALWQRLTFGEI

SEQ ID NO: 57 - protein, 26 aa, Artificial sequence
  Synthetic peptide
IEGPTLRQWLXRKXRXLWQRLTPGEI

SEQ ID NO: 58 - protein, 30 aa, Artificial sequence; identical to SEQ ID NO: 52
  Synthetic peptide

SEQ ID NO: 59 - protein, 30 aa, Artificial sequence; identical to SEQ ID NO: 52
  Synthetic peptide

SEQ ID NO: 60 - protein, 29 aa, Artificial sequence
  Synthetic peptide
IEGPTLRQWLAAFAKAFAALWQRLTPGEI

SEQ ID NO: 61 - protein, 29 aa, Artificial sequence
  Synthetic peptide
IEGPTLRQWLAAKAKAKAALWQRLTPGEI

SEQ ID NO: 62 - protein, 29 aa, Artificial sequence
  Synthetic peptide
IEGPTLKQWLAAKAKAKAALWQKLTPGEI

SEQ ID NO: 63 - protein, 27 aa, Artificial sequence
  Synthetic peptide
IEGPTLRQALAAXKXAALAQRLTPGEI

SEQ ID NO: 64 - protein, 29 aa, Artificial sequence
  Synthetic peptide
IEGPTLRQWLAARXKXRAALWQRLTPGEI

SEQ ID NO: 65 - protein, 29 aa, Artificial sequence
  Synthetic peptide
IEGPTLRQALAARXKXRAALAQRLTPGEI

SEQ ID NO: 66 - protein, 29 aa, Artificial sequence
  Synthetic peptide
IEGPTLRQALAARXKXRAALAQRLTPGEI

SEQ ID NO: 67 - protein, 29 aa, Artificial sequence
  Synthetic peptide
IEGPTLRQFLAARXKXRAALFQRLTPGEI

SEQ ID NO: 68 - protein, 29 aa, Artificial sequence
  Synthetic peptide
IEGPTLRQALAAKXKXKAALAQRLTPGEI

SEQ ID NO: 69 - protein, 29 aa, Artificial sequence
  Synthetic peptide
IEGPTLREALAAKXKXKAALAERLTPGEI

SEQ ID NO: 70 - protein, 29 aa, Artificial sequence
  Synthetic peptide
IEGPTLAQALAAKXKXKAALAQALTPGEI

SEQ ID NO: 71 - protein, 29 aa, Artificial sequence
  Synthetic peptide
IEGPTLAEALAAKXKXKAALAEALTPGEI

SEQ ID NO: 72 - protein, 29 aa, Artificial sequence
  Synthetic peptide
IEGPTLRQALAAXXKXXAALAQRLTPGEI

SEQ ID NO: 73 - protein, 29 aa, Artificial sequence
  Synthetic peptide
IEGPTLXQALAAXXKXXAALAQXLTPGEI

SEQ ID NO: 74 - protein, 25 aa, Artificial sequence
  Synthetic peptide
IEGPTLRQWLXAKXXLWQRLTPGEI

SEQ ID NO: 75 - protein, 27 aa, Artificial sequence
  Synthetic peptide
IEGPTLRQWLXARKXRXLWQRLTPGEI

SEQ ID NO: 76 - protein, 26 aa, Artificial sequence
  Synthetic peptide
ADGPTLREWIXAKXRXIWERLTPGDA

SEQ ID NO: 77 - protein, 25 aa, Artificial sequence
  Synthetic peptide
ADGPTLREWISFXADGPTLREWISP

SEQ ID NO: 78 - protein, 31 aa, Artificial sequence
  Synthetic peptide
CIEGPTLRQWLAARAKARAALWQRLTPGEIC

SEQ ID NO: 79 - protein, 31 aa, Artificial sequence; identical to SEQ ID NO: 48
  Synthetic peptide

SEQ ID NO: 80 - protein, 14 aa, Artificial Sequence
  Synthetic peptide
CADGPTLREWIXAC

(19) United States
(12) Patent Application Publication
(10) Pub. No.: US 2011/0071077 A1
(43) Pub. Date: Mar. 24, 2011

(54) Thrombopoietic Compounds

(75) Inventors: Janet L. Nichol, Oxnard, CA (US); James Reginald Kelly, JR., Sherman Oaks, CA (US); Mon-Gy Chen, Thousand Oaks, CA (US)

(73) Assignee: AMGEN INC., Thousand Oaks, CA (US)

(21) Appl. No.: 12/917,415

(22) Filed: Nov. 1, 2010

Related U.S. Application Data
(63) Continuation of application No. 11/698,502, filed on Jan. 25, 2007, now abandoned.
(60) Provisional application No. 60/761,874, filed on Jan. 25, 2006.

(51) Int. Cl. A61K 38/18; A61K 38/10; A61P 7/02; A61P 31/12; A61P 31/18; A61P 1/16; A61P 29/00

(52) U.S. Cl. 514/7.8

(57) ABSTRACT

The invention relates to the use of compounds, especially peptides or polypeptides, that have thrombopoietic activity, and pegylated forms thereof. The peptides and polypeptides of the invention may be used to increase platelets or platelet precursors (e.g., megakaryocytes) in a mammal.

### CROSS-REFERENCE TO RELATED APPLICATIONS

This application is a continuation of U.S. Ser. No. 11/698,502, filed 25 Jan. 2007, now pending, which claims priority to U.S. Provisional Patent Application Ser. No. 60/761,874, filed Jan. 25, 2006, both of which are hereby incorporated by reference.

### FIELD OF THE INVENTION

Generally, the invention relates to the field of compounds, especially peptides and polypeptides that have thrombopoietic activity. The compounds of the invention may be used to increase production of platelets or platelet precursors (e.g., megakaryocytes) in a mammal.

### BACKGROUND OF THE INVENTION

This invention relates to compounds, especially peptides that have the ability to stimulate in vitro and in vivo production of platelets and their precursor cells such as megakaryocytes. Before discussing the nature of the inventive compounds, the following is provided as a background regarding two proteins that have thrombopoietic activity: thrombopoietin (TPO) and megakaryocyte growth and development factor (MGDF).

The cloning of endogenous thrombopoietin (TPO) (Lok et al., Nature 369:568-571 (1994); Bartley et al., Cell 77:1117-1124 (1994); Kuter et al., Proc. Natl. Acad. Sci. USA 91:11104-11108 (1994); de Sauvage et al., Nature 369:533-538 (1994); Kato et al., Journal of Biochemistry 119:229-236 (1995); Chang et al., Journal of Biological Chemistry 270:511-514 (1995)) has rapidly increased our understanding of megakaryopoiesis (megakaryocyte production) and thrombopoiesis (platelet production).

Endogenous human TPO, a 60 to 70 kDa glycosylated protein primarily produced in the liver and kidney, consists of 332 amino acids (Bartley et al., Cell 77:1117-1124 (1994); Chang et al., Journal of Biological Chemistry 270:511-514 (1995)). The protein is highly conserved between different species, and has 23% homology with human erythropoietin (Gurney et al., Blood 85:981-988 (1995)) in the amino terminus (amino acids 1 to 172) (Bartley et al., Cell 77:1117-1124 (1994)). Endogenous TPO has been shown to possess all of the characteristics of the key biological regulator of thrombopoiesis. Its in vitro actions include specific induction of megakaryocyte colonies from both purified murine hematopoietic stem cells (Zeigler et al., Blood 84:4045-4052 (1994)) and human CD34⁺ cells (Lok et al., Nature 369:568-571 (1994); Rasko et al., Stem Cells 15:33-42 (1997)), the generation of megakaryocytes with increased ploidy (Broudy et al., Blood 85:402-413 (1995)), and the induction of terminal megakaryocyte maturation and platelet production (Zeigler et al., Blood 84:4045-4052 (1994); Choi et al., Blood 85:402-413 (1995)). Conversely, synthetic antisense oligodeoxynucleotides to the TPO receptor (c-Mpl) significantly inhibit the colony-forming ability of megakaryocyte progenitors (Methia et al., Blood 82:1395-1401 (1993)). Moreover, c-Mpl knock-out mice are severely thrombocytopenic and deficient in megakaryocytes (Alexander et al., Blood 87:2162-2170 (1996)).

Recombinant human MGDF (rHuMGDF, Amgen Inc., Thousand Oaks, Calif.) is another thrombopoietic polypeptide related to TPO. It is produced using E. coli transformed with a plasmid containing cDNA encoding a truncated protein encompassing the amino-terminal receptor-binding domain of human TPO (Ulich et al., Blood 86:971-976 (1995)). The polypeptide is extracted, refolded, and purified, and a poly[ethylene glycol] (PEG) moiety is covalently attached to the amino terminus. The resulting molecule is referred to herein as PEG-rHuMGDF or MGDF for short.

Various studies using animal models (Ulich, T. R. et al., Blood 86:971-976 (1995); Hokom, M. M. et al, Blood 86:4486-4492 (1995)) have clearly demonstrated the therapeutic efficacies of TPO and MGDF in bone marrow transplantation and in the treatment of thrombocytopenia, a condition that often results from chemotherapy or radiation therapy. Preliminary data in humans have confirmed the utility of MGDF in elevating platelet counts in various settings. (Basser et al., Lancet 348:1279-81 (1996); Kato et al., Journal of Biochemistry 119:229-236 (1995); Ulich et al., Blood 86:971-976 (1995)). MGDF might be used to enhance the platelet donation process, since administration of MGDF increases circulating platelet counts to about three-fold the original value in healthy platelet donors.

TPO and MGDF exert their action through binding to the c-Mpl receptor which is expressed primarily on the surface of certain hematopoietic cells, such as megakaryocytes, platelets, CD34⁺ cells and primitive progenitor cells (Debili, N. et al., Blood 85:391-401 (1995); de Sauvage, F. J. et al, Nature 369:533-538 (1994); Bartley, T. D., et al., Cell 77:1117-1124 (1994); Lok, S. et al., Nature 369: 565-8 (1994)). Like most receptors for interleukins and protein hormones, c-Mpl belongs to the class I cytokine receptor superfamily (Vigon, I. et al., Proc. Natl. Acad. Sci. USA 89:5640-5644 (1992)). Activation of this class of receptors involves ligand-binding induced receptor homodimerization which in turn triggers the cascade of signal transducing events.

In general, the interaction of a protein ligand with its receptor often takes place at a relatively large interface. However, as demonstrated in the case of human growth hormone bound to its receptor, only a few key residues at the interface actually contribute to most of the binding energy (Clackson, T. et al., Science 267:383-386 (1995)). This and the fact that the bulk of the remaining protein ligand serves only to display the binding epitopes in the right topology makes it possible to find active ligands of much smaller size.

In an effort toward this, the phage peptide library display system has emerged as a powerful technique in identifying small peptide mimetics of large protein ligands (Scott, J. K. et al., Science 249:386 (1990); Devlin, J. J. et al., Science 249:404 (1990)). By using this technique, small peptide molecules that act as agonists of the c-Mpl receptor were discovered (Cwirla, S. E. et al., Science 276:1696-1699 (1997)).

In such a study, random small peptide sequences displayed as fusions to the coat proteins of filamentous phage were affinity-eluted against the antibody-immobilized extracellular domain of c-Mpl and the retained phages were enriched for a second round of affinity purification. This binding selection and repropagation process was repeated many times to enrich the pool of tighter binders. As a result, two families of c-Mpl-binding peptides, unrelated to each other in their sequences, were first identified. Mutagenesis libraries were then created to further optimize the best binders, which finally led to the isolation of a very active peptide with an IC₅₀=2 nM and an EC₅₀=400 nM (Cwirla, S. E. et al., Science 276:1696-1699 (1997)). This 14-residue peptide, designated as a TMP (for TPO Mimetic Peptide), has no apparent sequence homology to TPO or MGDF. The structure of this TMP compound is as follows:

SEQ ID NO: 1
Ile Glu Gly Pro Thr Leu Arg Gln Trp Leu Ala
Ala Arg Ala
or
IEGPTLRQWLAARA

using single letter amino acid abbreviations.

Previously, in a similar study on EPO mimetic peptides, an EPO mimetic peptide (EMP) was discovered using the same technique (Wrighton, N. C. et al., Science 273:458-463 (1996)), and was found to act as a dimer in binding to the EPO receptor (EPOR). The (ligand)₂/(receptor)₂ complex thus formed had a C2 symmetry according to X-ray crystallographic data (Livnah, O. et al., Science 273:464-471 (1996)). Based on this structural information, a covalently linked dimer of EMP in which the C-termini of two EMP monomers were crosslinked with a flexible spacer was designed and found to have greatly enhanced binding as well as in vitro/in vivo bioactivity (Wrighton, N. C., et al., Nature Biotechnology 15:1261-1265 (1997)).

A similar C-terminal dimerization strategy was applied to the TPO mimetic peptide (TMP) (Cwirla, S. E. et al., Science 276:1696-1699 (1997)). It was found that a C-terminally linked dimer (C—C link) of TMP had an improved binding affinity of 0.5 nM and a remarkably increased in vitro activity (EC₅₀=0.1 nM) in cell proliferation assays (Cwirla, S. E. et al., Science 276:1696-1699 (1997)). The structure of this TMP C—C dimer is shown below:

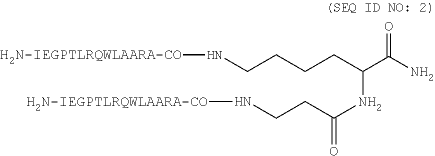

In another aspect of the present invention, the tandem dimers may be further attached to one or more moieties that are derived from immunoglobulin proteins, referred to generally as the Fc region of such immunoglobulins. The resulting compounds are referred to as Fc fusions of TMP tandem dimers.

The following is a brief background section relating to the Fc regions of antibodies that are useful in connection with some of the present compounds.

Antibodies comprise two functionally independent parts, a variable domain known as “Fab”, which binds antigen, and a constant domain, known as “Fc” which provides the link to effector functions such as complement fixation or phagocytosis. The Fc portion of an immunoglobulin has a long plasma half-life, whereas the Fab is short-lived. (Capon, et al., Nature 337:525-531 (1989)).

Therapeutic protein products have been constructed using the Fc domain to attempt to provide longer half-life or to incorporate functions such as Fc receptor binding, protein A binding, complement fixation, and placental transfer which all reside in the Fc region of immunoglobulins (Capon, et al., Nature 337:525-531 (1989)). For example, the Fc region of an IgG1 antibody has been fused to CD30-L, a molecule which binds CD30 receptors expressed on Hodgkin's Disease tumor cells, anaplastic lymphoma cells, T-cell leukemia cells and other malignant cell types. See, U.S. Pat. No. 5,480,981. IL-10, an anti-inflammatory and antirejection agent has been fused to murine Fcγ2a in order to increase the cytokine's short circulating half-life (Zheng, X. et al., The Journal of Immunology, 154: 5590-5600 (1995)). Studies have also evaluated the use of tumor necrosis factor receptor linked with the Fc protein of human IgG1 to treat patients with septic shock (Fisher, C. et al., N. Engl. J. Med., 334: 1697-1702 (1996); Van Zee, K. et al., The Journal of Immunology, 156: 2221-2230 (1996)). Fc has also been fused with CD4 receptor to produce a therapeutic protein for treatment of AIDS. See, Capon et al., Nature, 337:525-531 (1989). In addition, interleukin 2 has been fused to the Fc portion of IgG1 or IgG3 to overcome the short half life of interleukin 2 and its systemic toxicity. See, Harvill et al., Immunotechnology, 1: 95-105 (1995).

In spite of the availability of TPO and MGDF, there remains a need to provide additional compounds that have a biological activity of stimulating the production of platelets (thrombopoietic activity) and/or platelet precursor cells, especially megakaryocytes (megakaryopoietic activity). The present invention provides new compounds having such activity(ies), and related aspects.

### SUMMARY OF THE INVENTION

The present invention provides the use of compounds that are capable of binding to and triggering a transmembrane signal through, i.e., activating, the c-Mpl receptor, which is the same receptor that mediates the activity of endogenous thrombopoietin (TPO). Thus, the inventive compounds have thrombopoietic activity, i.e., the ability to stimulate, in vivo and in vitro, the production of platelets, and/or megakaryocytopoietic activity, i.e., the ability to stimulate, in vivo and in vitro, the production of platelet precursors.

In one embodiment, the invention includes the use of a compound that binds to an mpl receptor in the production of a medicament to stimulate megakaryocyte or platelet production, said compound formulated in a dosage amount of about 3 μg/kg to about 10 μg/kg and comprising the structure

TMP₁-(L₁)ₙ-TMP₂
wherein TMP₁ and TMP₂ are each independently selected from the group of core compounds comprising the structure:

X₂-X₃-X₄-X₅-X₆-X₇-X₈-X₉-X₁₀,
wherein,
X₂ is selected from the group consisting of Glu, Asp, Lys, and Val;
X₃ is selected from the group consisting of Gly and Ala;
X₄ is Pro;
X₅ is selected from the group consisting of Thr and Ser;
X₆ is selected from the group consisting of Leu, Ile, Val, Ala, and Phe;
X₇ is selected from the group consisting of Arg and Lys;
X₈ is selected from the group consisting of Gln, Asn, and Glu;
X₉ is selected from the group consisting of Trp, Tyr, Cys, Ala, and Phe;
X₁₀ is selected from the group consisting of Leu, Ile, Val, Ala, Phe, Met, and Lys;
L₁ is a linker; and
n is 0 or 1;
and physiologically acceptable salts thereof.

In one embodiment, L₁ comprises (Gly)ₙ, wherein n is 1 through 20, and when n is greater than 1, up to half of the Gly residues may be substituted by another amino acid selected from the remaining 19 natural amino acids or a stereoisomer thereof.

In addition to the core structure X₂-X₁₀ set forth above for TMP₁ and TMP₂, other related structures are also possible wherein one or more of the following is added to the TMP′ and/or TMP₂ core structure: X₁ is attached to the N-terminus and/or X₁₁, X₁₂, X₁₃, and/or X₁₄ are attached to the C-terminus, wherein X₁, X₁₂, X₁₃, and X₁₄ are as follows:
X₁ is selected from the group consisting of Ile, Ala, Val, Leu, Ser, and Arg;
X₁₁ is selected from the group consisting of Ala, Ile, Val, Leu, Phe, Ser, Thr, Lys, H is, and Glu;
X₁₂ is selected from the group consisting of Ala, Ile, Val, Leu, Phe, Gly, Ser, and Gln;
X₁₃ is selected from the group consisting of Arg, Lys, Thr, Val, Asn, Gln, and Gly; and
X₁₄ is selected from the group consisting of Ala, Ile, Val, Leu, Phe, Thr, Arg, Glu, and Gly.
In another preferred embodiment, the invention includes the use of such a compound wherein said TMP₁ and TMP₂ are independently selected form the group consisting of
X₂-X₃-X₄-X₅-X₅-X₇-X₈-X₉-X₁₀-X₁₁;
X₂-X₃-X₄-X₅-X₆-X₇-X₈-X₉-X₁₀-X₁₁-X₁₂;
X₂-X₃-X₄-X₅-X₆-X₇-X₈-X₉-X₁₀-X₁₁-X₁₂-X₁₃;
X₂-X₃-X₄-X₅-X₆-X₂-X₈-X₉-X₁₀-X₁₁-X₁₂-X₁₃-X₁₄;
X₁-X₂-X₃-X₄-X₅-X₆-X₇-X₈-X₉-X₁₀;
X₁-X₂-X₃-X₄-X₅-X₆-X₂-X₈-X₉-X₁₀-X₁₁;
X₁-X₂-X₃-X₄-X₅-X₆-X₇-X₈-X₉-X₁₀-X₁₁-X₁₂;
X₁-X₂-X₃-X₄-X₅-X₆-X₇-X₈-X₉-X₁₀-X₁₁-X₁₂-X₁₃; and
X₁-X₂-X₃-X₄-X₅-X₆-X₇-X₈-X₉-X₁₀-X₁₁-X₁₂-X₁₃-X₁₄,
wherein X₂-X₁₀ are as defined;
X₁ is selected from the group consisting of Ile, Ala, Val, Leu, Ser, and Arg;
X₁₁ is selected from the group consisting of Ala, Ile, Val, Leu, Phe, Ser, Thr, Lys, His, and Glu;
X₁₂ is selected from the group consisting of Ala, Ile, Val, Leu, Phe, Gly, Ser, and Gln;
X₁₃ is selected from the group consisting of Arg, Lys, Thr, Val, Asn, Gln, and Gly; and
X₁₄ is selected from the group consisting of Ala, Ile, Val, Leu, Phe, Thr, Arg, Glu, and Gly.

The invention also includes the use of such a compound wherein any of X₁, X₂, X₃, X₄, X₅, X₆, X₇, X₈, X₉, X₁₀, X₁₁, X₁₂, X₁₃, and X₁₄ is a non-naturally occurring amino acid.

The invention further includes the use of such a compound wherein said TMP₁ and/or TMP₂ are derivatized as set forth in one or more of the following:

one or more of the peptidyl [—C(O)NR-] linkages (bonds) have been replaced by a non-peptidyl linkage such as a —CH₂-carbamate linkage [—CH₂—OC(O)NR-]; a phosphonate linkage; a —CH₂-sulfonamide [—CH₂—S(O)₂NR-] linkage; a urea [—NHC(O)NH-] linkage; a —CH₂-secondary amine linkage; or an alkylated peptidyl linkage [—C(O)NR⁶— where R⁶ is lower alkyl];

the N-terminus is a —NRR¹ group; to a —NRC(O)R group; to a —NRC(O)OR group; to a —NRS(O)₂R group; to a —NHC(O)NHR group where R and R¹ are hydrogen and lower alkyl with the proviso that R and R¹ are not both hydrogen; to a succinimide group; to a benzyloxycarbonyl-NH—(CBZ—NH—) group; or to a benzyloxycarbonyl-NH— group having from 1 to 3 substituents on the phenyl ring selected from the group consisting of lower alkyl, lower alkoxy, chloro, and bromo;

the C terminus is —C(O)R² where R² is selected from the group consisting of lower alkoxy and —NR³R⁴ where R³ and R⁴ are independently selected from the group consisting of hydrogen and lower alkyl.

The invention includes such a compound wherein all amino acids in the compound have a D configuration. Likewise, the invention includes such a compound wherein at least one amino acid in the compound has a D configuration. Likewise, the invention includes such a compound wherein the compound is cyclic.

In one embodiment, the invention includes the use of a compound wherein TMP₁ and TMP₂ are each

(SEQ ID NO: 1)
Ile-Glu-Gly-Pro-Thr-Leu-Arg-Gln-Trp-Leu-Ala-Ala-
Arg-Ala..

The invention also includes the use of a compound wherein L₁ comprises a peptide. L₁ may also comprise Yₙ, wherein Y is a naturally-occurring amino acid or a stereoisomer thereof and n is 1 through 20. Likewise, L₁ may comprise (Gly)ₙ, wherein n is 1 through 20, and when n is greater than 1, up to half of the Gly residues may be substituted by another amino acid selected from the remaining 19 natural amino acids or a stereoisomer thereof. The invention also contemplates the use of a compound wherein L₁ is selected from the group consisting of

| | |
|---|---|
| (Gly)₃Lys(Gly)₄; | (SEQ ID NO: 6) |
| (Gly)₃AsnGlySer(Gly)₂; | (SEQ ID NO: 7) |
| (Gly)₃Cys(Gly)₄; | (SEQ ID NO: 8) |
| and | |
| GlyProAsnGly. | (SEQ ID NO: 9) |

The invention further comprises the use of a compound wherein L₁ comprises a Cys residue. The invention contemplates the use of such a compound when it is a dimer. They dimer may comprise the structure

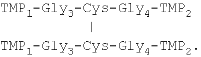

The invention further comprises the use of a compound wherein L₁ comprises (CH₂)ₙ, wherein n is 1 through 20.

In a further embodiment, the invention includes the use of compounds selected from the group consisting of

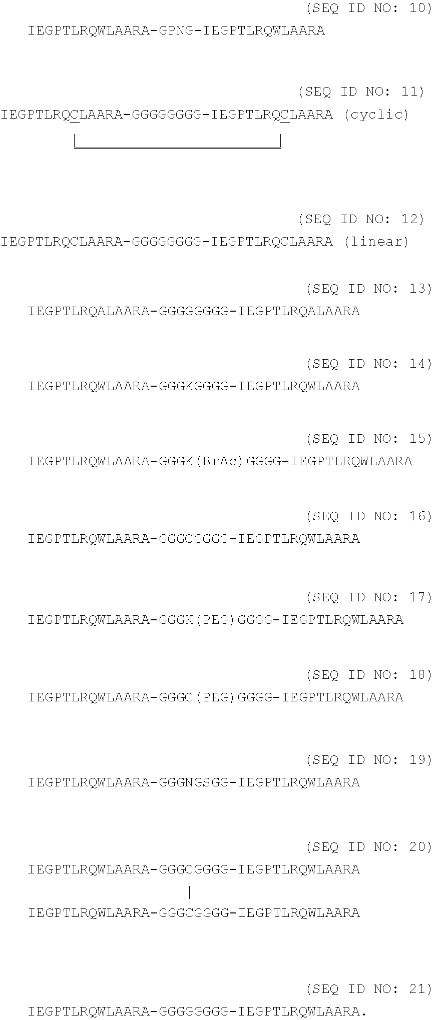

In another embodiment, the invention includes the use of compounds comprising the structure

(Fc)ₘ-(L₂)_{q}-TMP₁-(L₁)ₙ-TMP₂-(L₃)ᵣ-(Fc)ₚ

wherein L₁, L₂ and L₃ are linker groups which are each independently selected from the linker groups consisting of Yₙ, wherein Y is a naturally-occurring amino acid or a stereoisomer thereof and n is 1 through 20;

(Gly)ₙ, wherein n is 1 through 20, and when n is greater than 1, up to half of the Gly residues may be substituted by another amino acid selected from the remaining 19 natural amino acids or a stereoisomer thereof;

(Gly)₃Lys(Gly)₄ (SEQ ID NO: 6);

(Gly)₃AsnGlySer(Gly)₂ (SEQ ID NO: 7);

(Gly)₃Cys(Gly)₄ (SEQ ID NO: 8);

GlyProAsnGly (SEQ ID NO: 9);

a Cys residue; and

(CH₂)ₙ, wherein n is 1 through 20, and

wherein Fc is a constant region of an immunoglobulin; m, p, q and r are each independently selected from the group consisting of 0 and 1, wherein at least one of m or p is 1, and further wherein if m is 0 then q is 0, and if p is 0, then r is 0; and physiologically acceptable salts thereof.

The invention includes the use of such compounds wherein L₁, L₂ and L₃ are each independently selected from the group consisting of Yₙ, wherein Y is selected a naturally-occurring amino acid or a stereoisomer thereof and n is 1 through 20. The invention further includes the use of compounds wherein L₁ comprises (Gly)ₙ, wherein n is 1 through 20, and when n is greater than 1, up to half of the Gly residues may be substituted by another amino acid selected from the remaining 19 natural amino acids or a stereoisomer thereof. In addition, the invention includes the use of compounds wherein L₁, L₂ and L₃ are independently selected from the group consisting of

(Gly)₃Lys(Gly)₄ (SEQ ID NO: 6);

(Gly)₃AsnGlySer(Gly)₂ (SEQ ID NO: 7);

(Gly)₃Cys(Gly)₄ (SEQ ID NO: 8); and

GlyProAsnGly (SEQ ID NO: 9).

The invention also includes the use of a compound wherein L₁, L₂, or L₃ comprises a Cys residue or wherein L₁, L₂ or L₃ comprises (CH₂)ₙ, wherein n is 1 through 20.

In a further embodiment, the invention includes the use of the compounds as dimers.

In another embodiment, the invention includes the use of compounds have the general formula:

(Fc)ₘ-(L₂)_{q}-TMP₁-(L₁)ₙ-TMP₂-(L₃)ᵣ-(Fc)ₚ

wherein TMP₁, TMP₂ and n are each as described above; L₁, L₂ and L₃ are linker groups which are each independently selected from the linker groups described herein;

Fc is an Fc region of an immunoglobulin (as defined herein below); m, p, q and r are each independently selected from the group consisting of 0 and 1, wherein at least one of m or p is 1, and further wherein if m is 0 then q is 0, and if p is 0, then r is 0; and physiologically acceptable salts thereof. In one embodiment, L₁, L₂, and L₃ independently comprise (Gly)ₙ, wherein n is 1 through 20, and when n is greater than 1, up to half of the Gly residues may be substituted by another amino acid selected from the remaining 19 natural amino acids or a stereoisomer thereof.

Such a compound is selected from the group consisting of

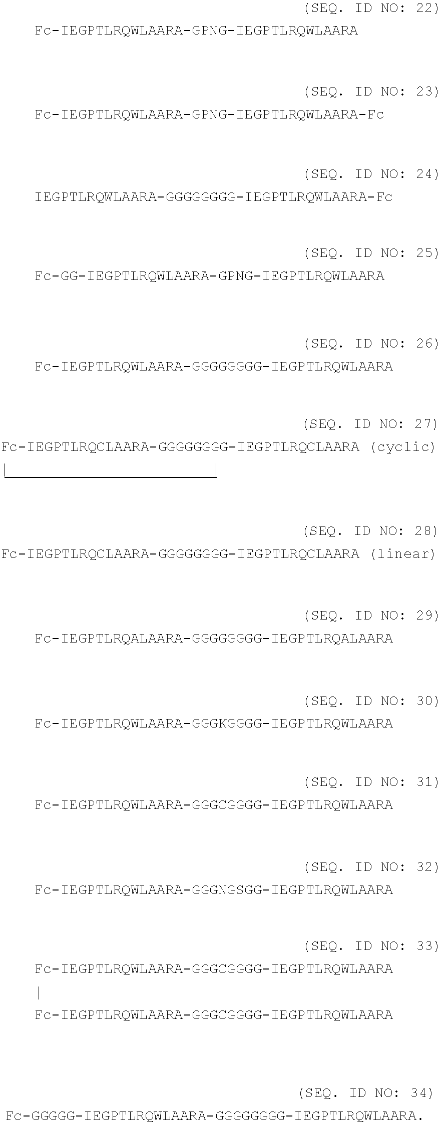

In yet another embodiment, the invention includes the use of a compound selected from the group consisting of

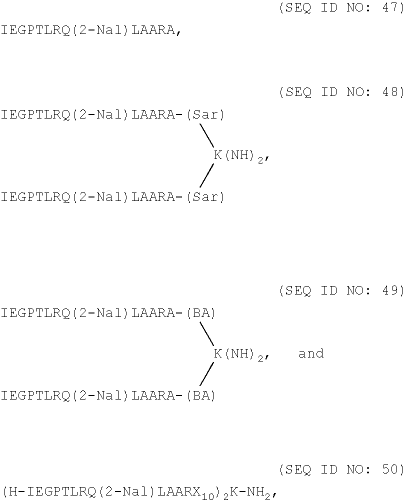

and pegylated forms thereof.

In a further embodiment, the invention includes the use of a compound selected from the group consisting of

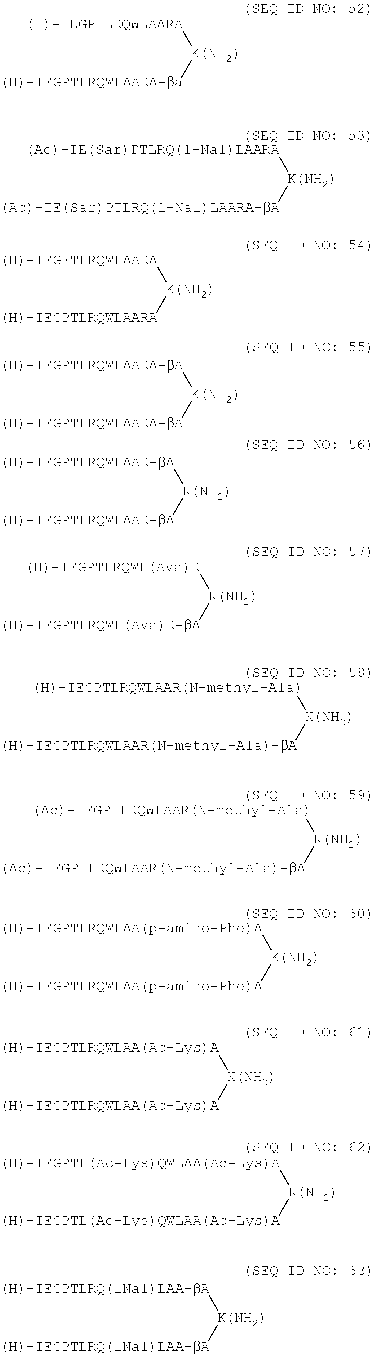

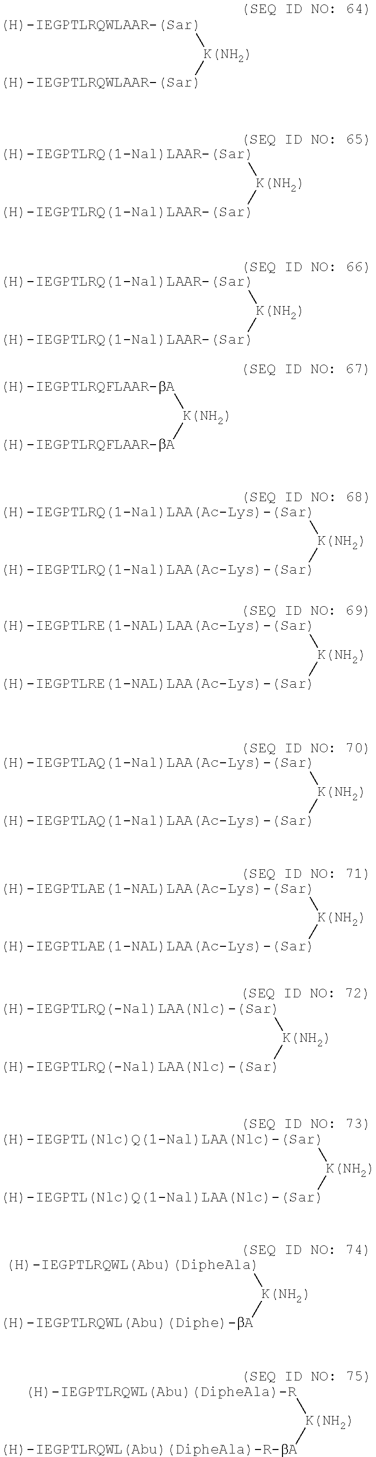

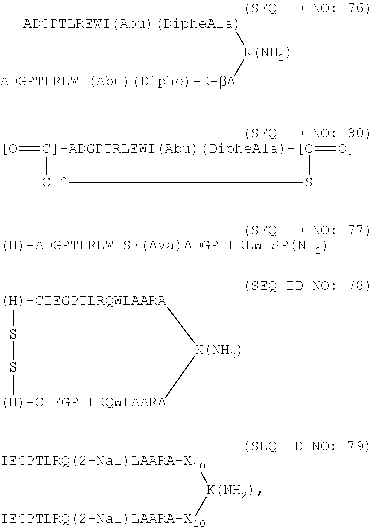

and pegylated forms thereof.

The compounds used in the invention are preferably peptides, and they may be prepared by standard synthetic methods or any other methods of preparing peptides. The compounds of this invention that encompass non-peptide portions may be synthesized by standard organic chemistry reactions, in addition to standard peptide chemistry reactions when applicable. However, the invention also includes the use of a compound wherein said compound is selected from the group consisting of an mpl-activating antibody; a microprotein comprising one or more mpl-binding sequences; a TPO mimetic sequence grafted into a human antibody framework; and a thrombopoietin synthebody.

Derivatives of any of the above compounds are also encompassed for use in the invention.

The invention includes the use of compounds for therapeutic or prophylactic purposes by incorporating them with appropriate pharmaceutical carrier materials and administering an effective amount to a subject, such as a human (or other mammal).

In a further embodiment, the invention includes the use of a compounds wherein said compound is formulated in amount to double megakaryocyte or platelet production over a baseline level. Such compounds may be formulated in amount to increase megakaryocyte or platelet production to a level of about 20×10⁹/L to about 2000×10⁹/L. Such compounds may also be formulated in amount to increase megakaryocyte or platelet production to a level of about 50×10⁹/L to about 250×10⁹/L. Likewise, the invention contemplates the use of such compounds formulated in amount to increase megakaryocyte or platelet production to a level of about 300×10⁹/L to about 1000×10⁹/L.

In another embodiment, the invention includes the use of the compounds described herein in the treatment of a hepatic disease or condition associated with thrombocytopenia. Such disease or condition treated includes, but is not limited to, alcoholic hepatitis, autoimmune hepatitis, drug-induced hepatitis, epidemic hepatitis, infectious hepatitis, long-incubation hepatitis, noninfectious hepatitis, serum hepatitis, short-incubation hepatitis, toxic hepatitis, transfusion hepatitis, viral hepatitis B (HBV), viral hepatitis C(HCV), viral hepatitis D (HDV), delta hepatitis, viral hepatitis E (HEV), viral hepatitis F (HFV), viral hepatitis G (HGV), liver disease, inflammation of the liver, and hepatic failure.

In yet another embodiment, the invention includes the treatment of thrombocytopenia resulting from the treatment of AIDS.

In a further embodiment, the invention includes a method of stimulating megakaryocyte or platelet production comprising administering a compound that binds to an mpl receptor in a dosage amount of about 3 μg/kg to about 10 μg/kg and comprising a structure TMP₁−(L₁)ₙ−TMP₂
wherein TMP₁ and TMP₂ are each independently selected from the group of core compounds comprising the structure:

X₂-X₃-X₄-X₅-X₆-X₇-X₈-X₉-X₁₀,
wherein,
X₂ is selected from the group consisting of Glu, Asp, Lys, and Val;
X₃ is selected from the group consisting of Gly and Ala;
X₄ is Pro;
X₅ is selected from the group consisting of Thr and Ser;
X₆ is selected from the group consisting of Leu, Ile, Val, Ala, and Phe;
X₇ is selected from the group consisting of Arg and Lys;
X₈ is selected from the group consisting of Gln, Asn, and Glu;
X₉ is selected from the group consisting of Trp, Tyr, Cys, Ala, and Phe;
X₁₀ is selected from the group consisting of Leu, Ile, Val, Ala, Phe, Met, and Lys;
L₁ is a linker; and
n is 0 or 1;
and physiologically acceptable salts thereof.

In one embodiment, L₁ comprises (Gly)ₙ, wherein n is 1 through 20, and when n is greater than 1, up to half of the Gly residues may be substituted by another amino acid selected from the remaining 19 natural amino acids or a stereoisomer thereof.

In addition to the core structure X₂-X₁₀ set forth above for TMP₁ and TMP₂, other related structures are also possible wherein one or more of the following is added to the TMP₁, and/or TMP₂ core structure: X₁ is attached to the N-terminus and/or X₁₁, X₁₂, X₁₃, and/or X₁₄ are attached to the C-terminus, wherein X₁, X₁₂, X₁₃, and X₁₄ are as follows:

X₁ is selected from the group consisting of Ile, Ala, Val, Leu, Ser, and Arg;
X₁₁ is selected from the group consisting of Ala, Ile, Val, Leu, Phe, Ser, Thr, Lys, H is, and Glu;
X₁₂ is selected from the group consisting of Ala, Ile, Val, Leu, Phe, Gly, Ser, and Gln;
X₁₃ is selected from the group consisting of Arg, Lys, Thr, Val, Asn, Gln, and Gly; and
X₁₄ is selected from the group consisting of Ala, Ile, Val, Leu, Phe, Thr, Arg, Glu, and Gly.

In another embodiment, the invention includes a compound wherein said TMP₁ and TMP₂ are independently selected form the group consisting of:

X₂-X₃-X₄-X₅-X₆-X₇-X₈-X₉-X₁₀-X₁₁;

X₂-X₃-X₄-X₅-X₆-X₇-X₈-X₉-X₁₀-X₁₁-X₁₂;

X₂-X₃-X₄-X₅-X₆-X₇-X₈-X₉-X₁₀-X₁₁-X₁₂-X₁₃;

X₂-X₃-X₄-X₅-X₆-X₇-X₈-X₉-X₁₀-X₁₁-X₁₂-X₁₃-X₁₄;

X₁-X₂-X₃-X₄-X₅-X₆-X₇-X₈-X₉-X₁₀;

X₁-X₂-X₃-X₄-X₅-X₆-X₇-X₈-X₉-X₁₀-X₁₁;

X₁-X₂-X₃-X₄-X₅-X₆-X₇-X₈-X₉-X₁₀-X₁₁-X₁₂;

X₁-X₂-X₃-X₄-X₅-X₆-X₇-X₈-X₉-X₁₀-X₁₁-X₁₂-X₁₃; and X₁-X₂-X₃-X₄-X₅-X₆-X₇-X₈-X₉-X₁₀-X₁₁-X₁₂-X₁₃-X₁₄,

wherein X₂-X₁₀ are as defined;

X₁ is selected from the group consisting of Ile, Ala, Val, Leu, Ser, and Arg;

X₁₁ is selected from the group consisting of Ala, Ile, Val, Leu, Phe, Ser, Thr, Lys, H is, and Glu;

X₁₂ is selected from the group consisting of Ala, Ile, Val, Leu, Phe, Gly, Ser, and Gln;

X₁₃ is selected from the group consisting of Arg, Lys, Thr, Val, Asn, Gln, and Gly; and

X₁₄ is selected from the group consisting of Ala, Ile, Val, Leu, Phe, Thr, Arg, Glu, and Gly.

The invention also includes a compound wherein any of X₁, X₂, X₃, X₄, X₅, X₆, X₇, X₈, X₉, X₁₀, X₁₁, X₁₂, X₁₃, and X₁₄ is a non-naturally occurring amino acid.

The invention further includes a compound wherein said TMP₁ and/or TMP₂ are derivatized as set forth in one or more of the following:

one or more of the peptidyl [—C(O)NR-] linkages (bonds) have been replaced by a non-peptidyl linkage such as a —CH₂-carbamate linkage [—CH₂—OC(O)NR-]; a phosphonate linkage; a —CH₂-sulfonamide [—CH₂—S(O)₂NR-] linkage; a urea [—NHC(O)NH-] linkage; a —CH₂-secondary amine linkage; or an alkylated peptidyl linkage [—C(O)NR⁶— where R⁶ is lower alkyl];

the N-terminus is a —NRR¹ group; to a —NRC(O)R group; to a —NRC(O)OR group; to a —NRS(O)₂R group; to a —NHC(O)NHR group where R and R¹ are hydrogen and lower alkyl with the proviso that R and R¹ are not both hydrogen; to a succinimide group; to a benzyloxycarbonyl-NH—(CBZ—NH—) group; or to a benzyloxycarbonyl-NH— group having from 1 to 3 substituents on the phenyl ring selected from the group consisting of lower alkyl, lower alkoxy, chloro, and bromo;

the C terminus is —C(O)R² where R² is selected from the group consisting of lower alkoxy and —NR³R⁴ where R³ and R⁴ are independently selected from the group consisting of hydrogen and lower alkyl.

The invention includes such a compound wherein all amino acids in the compound have a D configuration. Likewise, the invention includes such a compound wherein at least one amino acid in the compound has a D configuration. Likewise, the invention includes such a compound wherein the compound is cyclic.

In one embodiment, the invention includes a compound wherein TMP₁ and TMP₂ are each

Ile-Glu-Gly-Pro-Thr-Leu-Arg-Gln-Trp-Leu-Ala-Ala-Arg-Ala. (SEQ ID NO: 1).

The invention also includes a compound wherein L₁ comprises a peptide. L₁ may also comprise Yₙ, wherein Y is a naturally-occurring amino acid or a stereoisomer thereof and n is 1 through 20. Likewise, L₁ may comprise (Gly)ₙ, wherein n is 1 through 20, and when n is greater than 1, up to half of the Gly residues may be substituted by another amino acid selected from the remaining 19 natural amino acids or a stereoisomer thereof. The invention also contemplates the use of a compound wherein L₁ is selected from the group consisting of

(Gly)₃Lys(Gly)₄ (SEQ ID NO: 6);

(Gly)₃AsnGlySer(Gly)₂ (SEQ ID NO: 7);

(Gly)₃Cys(Gly)₄ (SEQ ID NO: 8); and

GlyProAsnGly (SEQ ID NO: 9).

The invention further comprises a compound wherein L₁ comprises a Cys residue. The invention contemplates the use of such a compound when it is a dimer. They dimer may comprise the structure

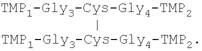

The invention further comprises a compound wherein L₁ comprises (CH₂)ₙ, wherein n is 1 through 20.

In a further embodiment, the invention includes a compound selected from the group consisting of

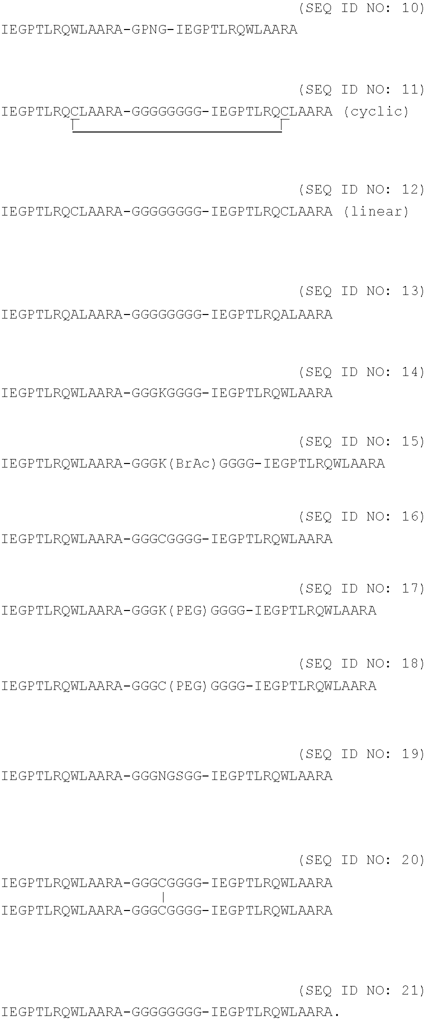

In another embodiment, the invention includes a compound comprising the structure

(Fc)ₘ-(L₂)_{q}-TMP₁-(L₁)ₙ-TMP₂-(L₃)ᵣ-(Fc)ₚ

wherein L₁, L₂ and L₃ are linker groups which are each independently selected from the linker groups consisting of

Yₙ, wherein Y is a naturally-occurring amino acid or a stereoisomer thereof and n is 1 through 20;

(Gly)ₙ, wherein n is 1 through 20, and when n is greater than 1, up to half of the Gly residues may be substituted by another amino acid selected from the remaining 19 natural amino acids or a stereoisomer thereof;

(Gly)₃Lys(Gly)₄ (SEQ ID NO: 6);

(Gly)₃AsnGlySer(Gly)₂ (SEQ ID NO: 7);

(Gly)₃Cys(Gly)₄ (SEQ ID NO: 8);

GlyProAsnGly (SEQ ID NO: 9);

a Cys residue; and

(CH₂)ₙ, wherein n is 1 through 20, and

wherein Fc is a constant region of an immunoglobulin; m, p, q and r are each independently selected from the group consisting of 0 and 1, wherein at least one of m or p is 1, and further wherein if m is 0 then q is 0, and if p is 0, then r is 0; and physiologically acceptable salts thereof.

The invention includes a compound wherein L₁, L₂ and L₃ are each independently selected from the group consisting of Yₙ, wherein Y is selected a naturally-occurring amino acid or a stereoisomer thereof and n is 1 through 20. The invention further includes the use of compounds wherein L₁ comprises (Gly)ₙ, wherein n is 1 through 20, and when n is greater than 1, up to half of the Gly residues may be substituted by another amino acid selected from the remaining 19 natural amino acids or a stereoisomer thereof. In addition, the invention includes the use of compounds wherein L₁, L₂ and L₃ are independently selected from the group consisting of

(Gly)₃Lys(Gly)₄ (SEQ ID NO: 6);

(Gly)₃AsnGlySer(Gly)₂ (SEQ ID NO: 7);

(Gly)₃Cys(Gly)₄ (SEQ ID NO: 8); and

GlyProAsnGly (SEQ ID NO: 9).

The invention also a compound wherein L₁, L₂, or L₃ comprises a Cys residue or wherein L₁, L₂ or L₃ comprises (CH₂)ₙ, wherein n is 1 through 20.

In a further embodiment, the invention includes a compound as a dimer.

In another embodiment, the invention includes a compound having the general formula:

(Fc)ₘ-(L₂)_{q}-TMP₁-(L₁)ₙ-TMP₂-(L₃)ᵣ-(Fc)ₚ

wherein TMP₁, TMP₂ and n are each as described above; L₁ L₂ and L₃ are linker groups which are each independently selected from the linker groups described herein;

Fc is an Fc region of an immunoglobulin (as defined herein below); m, p, q and r are each independently selected from the group consisting of 0 and 1, wherein at least one of m or p is 1, and further wherein if m is 0 then q is 0, and if p is 0, then r is 0; and physiologically acceptable salts thereof. In one embodiment, L₁, L₂, and L₃ independently comprise (Gly)ₙ, wherein n is 1 through 20, and when n is greater than 1, up to half of the Gly residues may be substituted by another amino acid selected from the remaining 19 natural amino acids or a stereoisomer thereof.

Such a compound is selected from the group consisting of

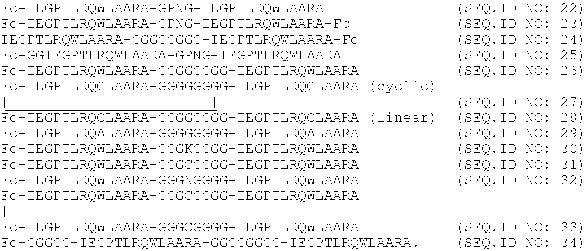

In yet another embodiment, the invention includes a compound selected from the group consisting of

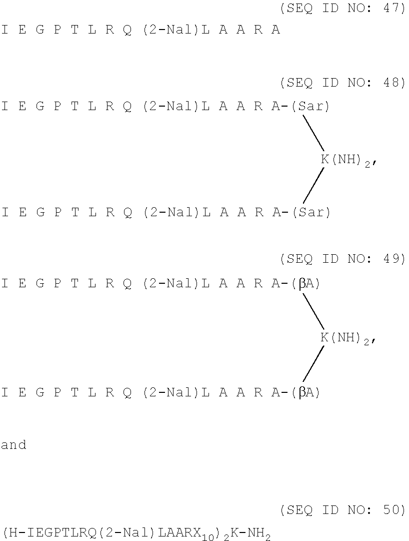

and pegylated forms thereof.

In a further embodiment, the invention includes a compound selected from the group consisting of

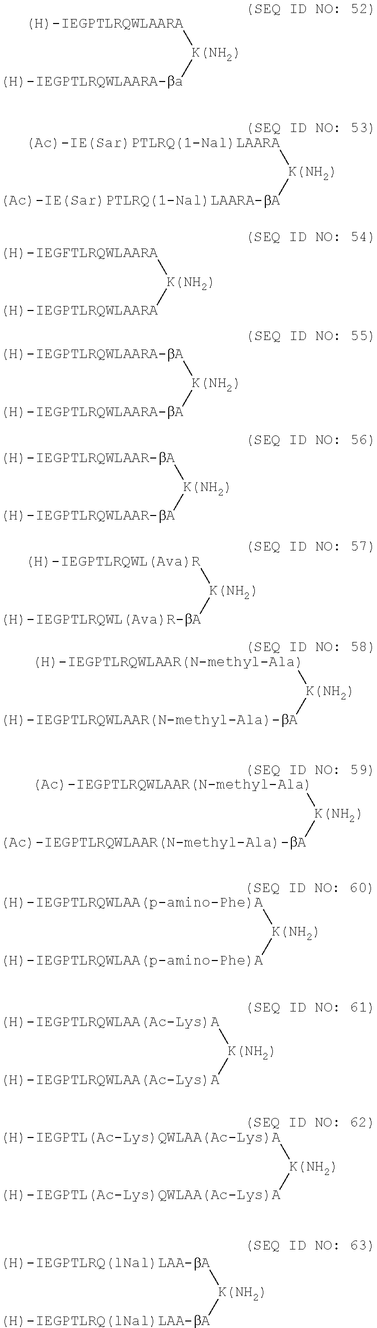

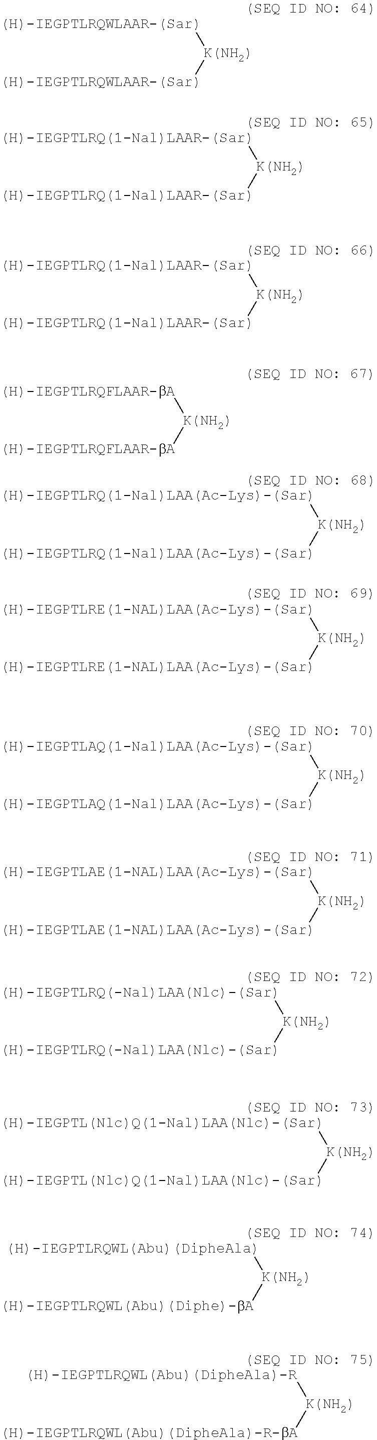

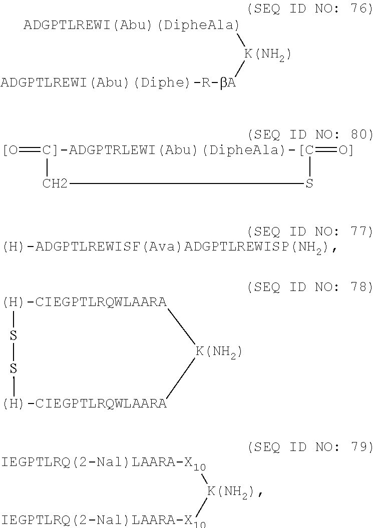

and pegylated forms thereof.

The compounds used in the methods of the invention are preferably peptides, and they may be prepared by standard synthetic methods or any other methods of preparing peptides. The compounds of this invention that encompass non-peptide portions may be synthesized by standard organic chemistry reactions, in addition to standard peptide chemistry reactions when applicable. However, the invention also includes the use of a compound wherein said compound is selected from the group consisting of an mpl-activating antibody; a microprotein comprising one or more mpl-binding sequences; a TPO mimetic sequence grafted into a human antibody framework; and a thrombopoietin synthebody.

Derivatives of any of the above compounds are also encompassed for use in the methods of the invention.

The invention includes compounds for therapeutic or prophylactic purposes by incorporating them with appropriate pharmaceutical carrier materials and administering an effective amount to a subject, such as a human (or other mammal).

In a further embodiment, the invention includes methods of stimulating megakaryocyte or platelet production comprising administering a compound wherein said compound is administered in a dosage amount effective to double megakaryocyte or platelet production over a baseline level. Such compounds may be administered in a dosage amount effective to increase megakaryocyte or platelet production to a level of about 20×10⁹/L to about 2000×10⁹/L. Such compounds may also be administered in a dosage amount effective to increase megakaryocyte or platelet production to a level of about 50×10⁹/L to about 250×10⁹/L. Likewise, the invention contemplates that compounds are administered in a dosage amount effective in amount to increase megakaryocyte or platelet production to a level of about 300×10⁹/L to about 1000×10⁹/L.

In another embodiment, the invention includes methods of stimulating megakaryocyte or platelet production described herein in the treatment of a hepatic disease or condition associated with thrombocytopenia. Such disease or condition treated includes, but is not limited to, alcoholic hepatitis, autoimmune hepatitis, drug-induced hepatitis, epidemic hepatitis, infectious hepatitis, long-incubation hepatitis, noninfectious hepatitis, serum hepatitis, short-incubation hepatitis, toxic hepatitis, transfusion hepatitis, viral hepatitis B (HBV), viral hepatitis C(HCV), viral hepatitis D (HDV), delta hepatitis, viral hepatitis E (HEV), viral hepatitis F (HFV), viral hepatitis G (HGV), liver disease, inflammation of the liver, and hepatic failure.

In yet another embodiment, the invention includes methods of stimulating megakaryocyte or platelet production in the treatment of thrombocytopenia resulting from the treatment of AIDS.

Other related aspects are also included in the instant invention.

### BRIEF DESCRIPTION OF THE FIGURES

Numerous other aspects and advantages of the present invention will therefore be apparent upon consideration of the following detailed description thereof, reference being made to the drawings wherein:

FIG. 1 shows exemplary Fc polynucleotide and protein sequences (SEQ ID NO: 3 is the coding strand reading 5′→3′, SEQ ID NO: 4 is the complementary strand reading 3′→5′; and SEQ ID NO: 5 is the encoded amino acids sequence) of human IgG1 that may be used in the Fc fusion compounds of this invention.

FIG. 2 shows a synthetic scheme for the preparation of pegylated peptide 19 (SEQ ID NO:17).

FIG. 3 shows a synthetic scheme for the preparation of pegylated peptide 20 (SEQ ID NO:18).

FIG. 4 shows the number of platelets generated in vivo in normal female BDF1 mice treated with one 100 μg/kg bolus injection of various compounds, as follows: PEG-MGDF means 20 kD average molecular weight PEG attached to the N-terminal amino group by reductive amination of amino acids 1-163 of native human TPO, which is expressed in E. coli (so that it is not glycosylated) (See WO 95/26746 entitled “Compositions and Methods for Stimulating Megakaryocyte Growth and Differentiation”); TMP means the compound of SEQ ID NO: 1; TMP-TMP means the compound of SEQ ID NO: 21; PEG-TMP-TMP means the compound of SEQ ID NO: 18, wherein the PEG group is a 5 kD average molecular weight PEG attached as shown in FIG. 3; TMP-TMP-Fc is defined herein below and Fc-TMP-TMP is the same as TMP-TMP-Fc except that the Fc group is attached at the N-terminal end rather than the C-terminal end of the TMP-TMP peptide.

FIG. 5 shows the number of platelets generated in vivo in normal BDF1 mice treated with various compounds delivered via implanted osmotic pumps over a 7-day period. The compounds are defined in the same manner as set forth above for FIG. 4.

FIGS. 6A, 6B, and 6C show schematic diagrams of preferred compounds of the present invention. FIG. 6A shows an Fc fusion compound wherein the Fc moiety is fused at the N-terminus of the TMP dimer, and wherein the Fc portion is a monomeric (single chain) form. FIG. 6B shows an Fc fusion compound wherein the Fc region is fused at the N-terminus of the TMP dimer, and wherein the Fc portion is dimeric, and one Fc monomer is attached to a TMP dimer. FIG. 6C shows an Fc fusion compound wherein the Fc moiety is fused at the N-terminus of the TMP dimer, and wherein the Fc portion is dimeric and each Fc monomer is attached to a TMP dimer.

FIG. 7 illustrates the study schemas for Part A (top) and Part B (bottom). Arrows indicate treatment days.

FIG. 8 shows peak individual platelet counts by dose and cohort for Part A. The baseline platelet count and the peak platelet count after dose 1 and dose 2 are indicated. Three patients did not receive a second dose. The shaded area shows the target platelet count response. Platelet counts associated with rescue medication are excluded.

FIG. 9 shows peak platelet count by treatment group in Part B. Platelet counts associated with rescue medication are excluded. Platelet counts are rounded to the nearest 10 for display purposes. Unrounded platelet medians appear as dashes within treatments. The shaded area shows the target platelet response.

FIG. 10 shows platelet counts over time in responding patients (top) and non-responding patients (bottom) treated with an AMP2 molecule or placebo (weekly treatment for 6 weeks). Placebo patients are indicated by a bold red line. The shaded area shows the target platelet count response. Platelet counts associated with rescue medication are excluded.

FIG. 11 provides additional compounds included in the present invention.

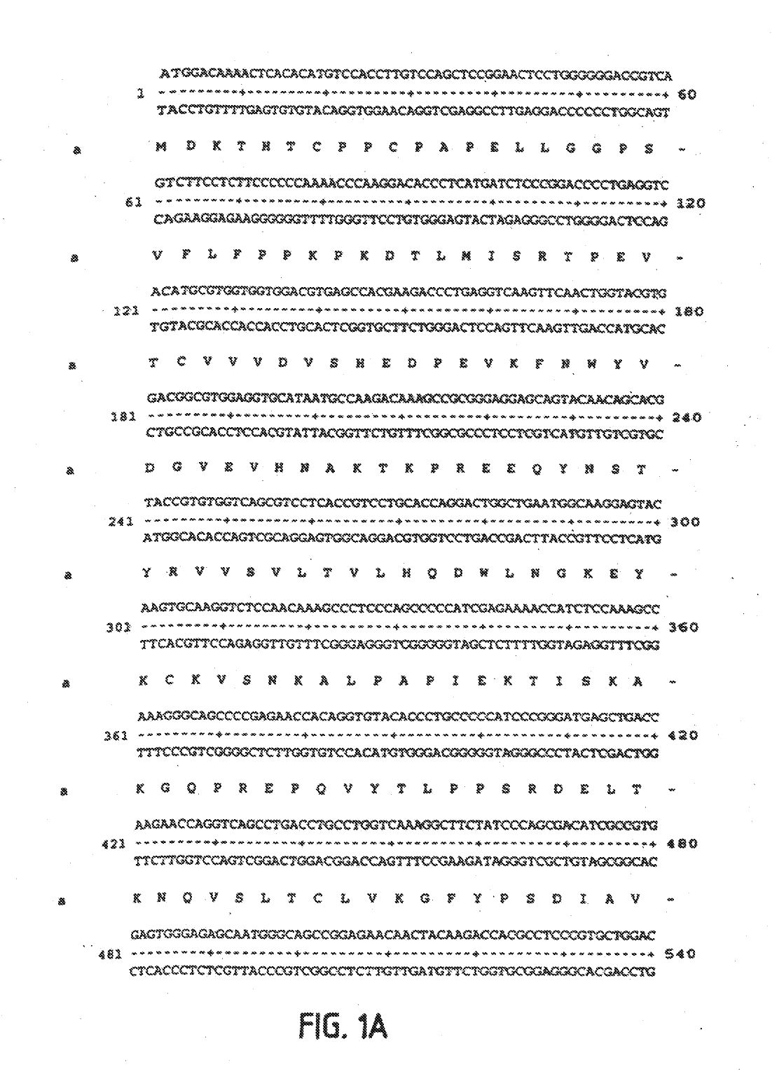

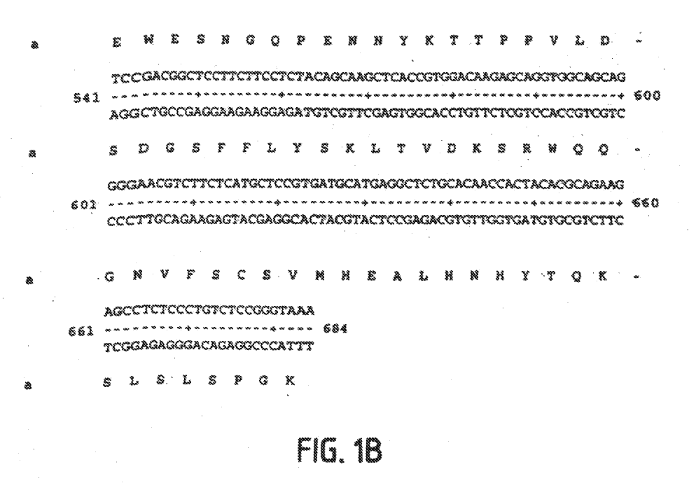

### DETAILED DESCRIPTION OF THE PREFERRED EMBODIMENTS

In an effort to seek small structures as lead compounds in the development of therapeutic agents with more desirable properties, a different type of dimer of TMP and related structures were designed in which the C-terminus of one TMP peptide was linked to the N-terminus of a second TMP peptide, either directly or via a linker and the effects of this dimerization strategy on the bioactivity of the resulting dimeric molecules were then investigated. In some cases, these so-called tandem dimers (C—N link) were designed to have linkers between the two monomers, the linkers being preferably composed of natural amino acids, therefore rendering their synthesis accessible to recombinant technologies.

The present invention is based on the discovery of a group of compounds that have thrombopoietic activity and which are thought to exert their activity by binding to the endogenous TPO receptor, c-Mpl.

### Compounds and Derivatives

In a first preferred embodiment, the inventive compounds comprise the following general structure:

TMP₁−(L₁)ₙ−TMP₂
wherein TMP₁ and TMP₂ are each independently selected from the group of compounds comprising the core structure:

X₂-X₃-X₄-X₅-X₆-X₇-X₈-X₉-X₁₀,
wherein,
X₂ is selected from the group consisting of Glu, Asp, Lys, and Val;
X₃ is selected from the group consisting of Gly and Ala;
X₄ is Pro;
X₅ is selected from the group consisting of Thr and Ser;
X₆ is selected from the group consisting of Leu, Ile, Val, Ala, and Phe;
X₇ is selected from the group consisting of Arg and Lys;
X₈ is selected from the group consisting of Gln, Asn, and Glu;
X₉ is selected from the group consisting of Trp, Tyr, Cys, Ala, and Phe;
X₁₀ is selected from the group consisting of Leu, Ile, Val, Ala, Phe, Met, and Lys;
L₁ is a linker as described herein; and
n is 0 or 1; and physiologically acceptable salts thereof.

In one embodiment, L₁, comprises (Gly)ₙ, wherein n is 1 through 20, and when n is greater than 1, up to half of the Gly residues may be substituted by another amino acid selected from the remaining 19 natural amino acids or a stereoisomer thereof.

In addition to the core structure X₂-X₁₀ set forth above for TMP₁ and TMP₂, other related structures are also possible wherein one or more of the following is added to the TMP₁ and/or TMP₂ core structure: X₁ is attached to the N-terminus and/or X₁₁, X₁₂, X₁₃, and/or X₁₄ are attached to the C-terminus, wherein X₁, X₁₁, X₁₂, X₁₃, and X₁₄ are as follows:

X₁ is selected from the group consisting of Ile, Ala, Val, Leu, Ser, and Arg;

X₁₁ is selected from the group consisting of Ala, Ile, Val, Leu, Phe, Ser, Thr, Lys, His, and Glu;

X₁₂ is selected from the group consisting of Ala, Ile, Val, Leu, Phe, Gly, Ser, and Gln;

X₁₃ is selected from the group consisting of Arg, Lys, Thr, Val, Asn, Gln, and Gly; and X₁₄ is selected from the group consisting of Ala, Ile, Val, Leu, Phe, Thr, Arg, Glu, and Gly.

The term “TMP” is used to mean a moiety made up of, i.e., comprising, at least 9 subunits (X₂-X₁₀), wherein X₂-X₁₀ comprise the core structure. The X₂-X₁₄ subunits are preferably amino acids independently selected from among the 20 naturally-occurring amino acids, however, the invention embraces compounds where X₂-X₁₄ are independently selected from the group of atypical, non-naturally occurring amino acids well known in the art.

The invention includes TMPs which comprise naturally occurring or non-naturally occurring amino acids. Such non-naturally occurring amino acids include, but are not limited to, norleucine, α-alanine, sarcosine, and b-(2-naphthyl)alanine. For instance, naphthylalanine can be substituted for tryptophan, facilitating synthesis. Other synthetic amino acids that can be substituted into the peptides of the present invention include L-hydroxypropyl, L-3,4-dihydroxyphenylalanyl, d amino acids such as L-d-hydroxylysyl and D-d-methylalanyl, L-□-methylalanyl, γamino acids, and isoquinolyl. D amino acids and non-naturally occurring synthetic amino acids can also be incorporated into the peptides of the present invention (see, e.g., Roberts, et al., Unusual Amino/Acids in Peptide Synthesis, 5(6):341-449 (1983)).

The invention also includes the replacement of naturally occurring side chains of the 20 genetically encoded amino acids (or D amino acids) with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered hetereocyclic. In particular, proline analogs in which the ring size of the proline residue is changed from 5 members to 4, 6, or 7 members can be employed. Cyclic groups can be saturated or unsaturated, and if unsaturated, can be aromatic or non-aromatic. Heterocyclic groups preferably contain one or more nitrogen, oxygen, and/or sulphur heteroatoms. Examples of such groups include the furazanyl, furyl, imidazolidinyl, imidazolyl, imidazolinyl, isothiazolyl, isoxazolyl, morpholinyl (e.g. morpholino), oxazolyl, piperazinyl (e.g. I-piperazinyl), piperidyl (e.g. I-piperidyl, piperidino), pyranyl, pyrazinyl, pyrazolidinyl, pyrazolinyl, pyrazolyl, pyridazinyl, pyridyl, pyrimidinyl, pyrrolidinyl (e.g. I-pyrrolidinyl), pyrrolinyl, pyrrolyl, thiadiazolyl, thiazolyl, thienyl, thiomorpholinyl (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl.

Specific preferred amino acids are identified for each position. For example, X₂ may be Glu, Asp, Lys, or Val. Both three-letter and single letter abbreviations for amino acids are used herein; in each case, the abbreviations are the standard ones used for the 20 naturally-occurring amino acids or well-known variations thereof.

These amino acids may have either L or D stereochemistry (except for Gly, which is neither L nor D), and the TMPs may comprise a combination of stereochemistries. However, the L stereochemistry is preferred for all of the amino acids in the TMP chain. The invention also provides reverse TMP molecules wherein the amino terminal to carboxy terminal sequence of the amino acids is reversed. For example, the reverse of a molecule having the normal sequence X₁-X₂-X₃ would be X₃-X₂-X₁. The invention also provides retro-reverse TMP molecules wherein, like a reverse TMP, the amino terminal to carboxy terminal sequence of amino acids is reversed and residues that are normally “L” enantiomers in TMP are altered to the “D” stereoisomer form.

AMP2 is one of the TMP molecules of the invention.

In one embodiment, the invention also includes peptide compounds wherein from zero to all of the—C(O)NH—linkages of the peptide have been replaced by a linkage selected from the group consisting of a—CH₂OC(O)NR—linkage; a phosphonate linkage; a—CH₂S(O)₂NR—linkage; a—CH₂2NR—linkage; and a—C(O)NR⁶-linkage; and a—NHC(O)NH—linkage where R is hydrogen or lower alkyl and R⁶ is lower alkyl, further wherein the N-terminus of said peptide or peptide mimetic is selected from the group consisting of a—NRR¹ group; a—NRC(O)R group; a—NRC(O)OR group; a—NRS(O)₂R group; a—NHC(O)NHR group; a succinimide group; a benzyloxycarbonyl-NH—group; and a benzyloxycarbonyl-NH—group having from 1 to 3 substituents on the phenyl ring selected from the group consisting of lower alkyl, lower alkoxy, chloro, and bromo, where R and R¹ are independently selected from the group consisting of hydrogen and lower alkyl, and still further wherein the C-terminus of said peptide or peptide mimetic has the formula—C(O)R² where R² is selected from the group consisting of hydroxy, lower alkoxy, and —NR³R⁴ where R³ and R⁴ are independently selected from the group consisting of hydrogen and lower alkyl and where the nitrogen atom of the—NR.sup.3R4 group can optionally be the amine group of the N-terminus of the peptide so as to form a cyclic peptide, and physiologically acceptable salts thereof.

Additionally, physiologically acceptable salts of the TMPs are also encompassed. “Physiologically acceptable salts” means any salts that are known or later discovered to be pharmaceutically acceptable. Some specific preferred examples are: acetate, trifluoroacetate, hydrochloride, hydrobromide, sulfate, citrate, tartrate, glycolate, oxalate.

It is also contemplated that “derivatives” of the TMPs may be substituted for the above-described TMPs. Such derivative TMPs include moieties wherein one or more of the following modifications have been made:

one or more of the peptidyl [—C(O)NR-] linkages (bonds) have been replaced by a non-peptidyl linkage such as a —CH₂-carbamate linkage [—CH₂—OC(O)NR-]; a phosphonate linkage; a —CH₂-sulfonamide [—CH₂—S(O)₂NR-] linkage; a urea [—NHC(O)NH-] linkage; a —CH₂-secondary amine linkage; or an alkylated peptidyl linkage [—C(O)NR⁶— where R⁶ is lower alkyl];

peptides wherein the N-terminus is derivatized to a —NRR¹ group; to a —NRC(O)R group; to a —NRC(O)OR group; to a —NRS(O)₂R group; to a —NHC(O)NHR group, where R and R¹ are hydrogen or lower alkyl, with the proviso that R and R¹ are not both hydrogen; to a succinimide group; to a benzyloxycarbonyl-NH— (CBZ—NH—) group; or to a benzyloxycarbonyl-NH— group having from 1 to 3 substituents on the phenyl ring selected from the group consisting of lower alkyl, lower alkoxy, chloro, and bromo; and

peptides wherein the free C terminus is derivatized to —C(O)R² where R² is selected from the group consisting of lower alkoxy and —NR³R⁴ where R³ and R⁴ are independently selected from the group consisting of hydrogen and lower alkyl. By “lower” is meant a group having from 1 to 6 carbon atoms.

Additionally, modifications of individual amino acids may be introduced into the TMP molecule by reacting targeted amino acid residues of the peptide with an organic derivatizing agent that is capable of reacting with selected side chains or terminal residues. The following are exemplary:

Lysinyl and amino terminal residues may be reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues may be modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine guanidino group.

The specific modification of tyrosyl residues per se has been studied extensively, with particular interest in introducing spectral labels into tyrosyl residues by reaction with aromatic diazonium compounds or tetranitromethane. Most commonly, N-acetylimidizole and tetranitromethane may be used to form O-acetyl tyrosyl species and 3-nitro derivatives, respectively.

Carboxyl side groups (aspartyl or glutamyl) may be selectively modified by reaction with carbodiimides (R′—N═C═N—R′) such as 1-cyclohexyl-3-(2-morpholinyl-(4-ethyl) carbodiimide or 1-ethyl-3-(4-azonia-4,4-dimethylpentyl) carbodiimide. Furthermore, aspartyl and glutamyl residues may be converted to asparaginyl and glutaminyl residues by reaction with ammonium ions.

Glutaminyl and asparaginyl residues are frequently deamidated to the corresponding glutamyl and aspartyl residues. Alternatively, these residues may be deamidated under mildly acidic conditions. Either form of these residues falls within the scope of this invention.

Derivatization with bifunctional agents is useful for cross-linking the peptides or their functional derivatives to a water-insoluble support matrix or to other macromolecular carriers. Commonly used cross-linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis (succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 may be employed for protein immobilization.

Other possible modifications include hydroxylation of proline and lysine, phosphorylation of hydroxyl groups of seryl or threonyl residues, oxidation of the sulfur atom in Cys, methylation of the alpha-amino groups of lysine, arginine, and histidine side chains (Creighton, T. E., Proteins: Structure and Molecule Properties, W. H. Freeman & Co., San Francisco, pp. 79-86 (1983)), acetylation of the N-terminal amine, and, in some instances, amidation of the C-terminal carboxyl groups.

Such derivatized moieties preferably improve one or more characteristics including thrombopoietic activity, solubility, absorption, biological half life, and the like of the inventive compounds. Alternatively, derivatized moieties result in compounds that have the same, or essentially the same, characteristics and/or properties of the compound that is not derivatized. The moieties may alternatively eliminate or attenuate any undesirable side effect of the compounds and the like.

In addition to the core structure set forth above, X₂-X₁₀, other structures that are specifically contemplated are those in which one or more additional X groups are attached to the core structure. Thus, X₁, and/or X₁₁, X₁₂, X₁₃, and X₁₄ may be attached to the core structure. Some exemplary additional structures are the following:

X₂-X₃-X₄-X₅-X₆-X₇-X₈-X₉-X₁₀-X₁₁;

X₂-X₃-X₄-X₅-X₆-X₇-X₈-X₉-X₁₀-X₁₁-X₁₂;

X₂-X₃-X₄-X₅-X₆-X₇-X₈-X₉-X₁₀-X₁₁-X₁₂-X₁₃;

X₂-X₃-X₄-X₅-X₆-X₇-X₃-X₉-X₁₀-X₁₁-X₁₂-X₁₃-X₁₄;

X₁-X₂-X₃-X₄-X₅-X₆-X₇-X₈-X₉-X₁₀;

X₁-X₂-X₃-X₄-X₅-X₆-X₇-X₈-X₉-X₁₀-X₁₁;

X₁-X₂-X₃-X₄-X₅-X₆-X₇-X₈-X₉-X₁₀-X₁₁-X₁₂;

X₁-X₂-X₃-X₄-X₅-X₆-X₇-X₈-X₉-X₁₀-X₁₁-X₁₂-X₁₃;

X₁-X₂-X₃-X₄-X₅-X₆-X₇-X₈-X₉-X₁₀-X₁₁-X₁₂-X₁₃-X₁₄,

wherein X₁ through X₁₄ are as described above. Each of TMP₁ and TMP₂ may be the same or different in sequence and/or length. In some preferred embodiments, TMP₁ and TMP₂ are the same.

A particularly preferred TMP is the following:

Ile-Glu-Gly-Pro-Thr-Leu-Arg-Gln-Trp-Leu-Ala-Ala-Arg-Ala (SEQ ID NO:1).

As used herein “comprising” means, inter alia, that a compound may include additional amino acids on either or both of the—or C-termini of the given sequence. However, as long as a structure such as X₂ to X₁₀, X₁ to X₁₄, or one of the other exemplary structures is present, the remaining chemical structure is relatively less important. Of course, any structure outside of the core X₂ to X₁₀ structure, or the X₁ to X₁₄, structure, should not significantly interfere with thrombopoietic activity of the compound. For example, an N-terminal Met residue is envisioned as falling within this invention. Additionally, although many of the preferred compounds of the invention are tandem dimers in that they possess two TMP moieties, other compounds of this invention are tandem multimers of the TMPs, i.e., compounds of the following exemplary structures:

TMP₁-L-TMP₂-L-TMP₃;

TMP₁-L-TMP₂-L-TMP₃-L-TMP₄;

TMP₁-L-TMP₂-L-TMP₃-L-TMP₄-L-TMP₅;

wherein TMP₁, TMP₂, TMP₃, TMP₄, and TMP₅ can have the same or different structures, and wherein each TMP and L is defined as set forth herein, and the linkers are each optional. Preferably, the compounds of this invention will have from 2-5 TMP moieties, particularly preferably 2-3, and most preferably 2. The compounds of the first embodiment of this invention will preferably have less than about 60, more preferably less than about 40 amino acids in total (i.e., they will be peptides).

As noted above, the compounds of the first embodiment of this invention are preferably TMP dimers which are either bonded directly or are linked by a linker group. The monomeric TMP moieties are shown in the conventional orientation from N to C terminus reading from left to right. Accordingly, it can be seen that the inventive compounds are all oriented so that the C terminus of TMP₁ is attached either directly or through a linker to the N-terminus of TMP₂. This orientation is referred to as a tandem orientation, and the inventive compounds may be generally referred to as “tandem dimers”. These compounds are referred to as dimers even if TMP₁ and TMP₂ are structurally distinct. That is, both homodimers and heterodimers are envisioned.

The “linker” group (“L₁”) is optional. When it is present, it is not critical what its chemical structure is, since it serves primarily as a spacer. The linker should be chosen so as not to interfere with the biological activity of the final compound and also so that immunogenicity of the final compound is not significantly increased. The linker is preferably made up of amino acids linked together by peptide bonds. Thus, in preferred embodiments, the linker comprises Yₙ, wherein Y is a naturally occurring amino acid or a steroisomer thereof and “n” is any one of 1 through 20. The linker is therefore made up of from 1 to 20 amino acids linked by peptide bonds, wherein the amino acids are selected from the 20 naturally-occurring amino acids. In a more preferred embodiment, the 1 to 20 amino acids are selected from Gly, Ala, Pro, Asn, Gln, Cys, Lys. Even more preferably, the linker is made up of a majority of amino acids that are sterically un-hindered, such as Gly, Gly-Gly [(Gly)₂], Gly-Gly-Gly [(Gly)₃] . . . (Gly)₂₀, Ala, Gly-Ala, Ala-Gly, Ala-Ala, etc. Other specific examples of linkers are:

(Gly)₃Lys(Gly)₄ (SEQ ID NO: 6);

(Gly)₃AsnGlySer(Gly)₂ (SEQ ID NO: 7)

(this structure provides a site for glycosylation, when it is produced recombinantly in a mammalian cell system that is capable of glycosylating such sites);

(Gly)₃Cys(Gly)₄ (SEQ ID NO: 8); and

GlyProAsnGly (SEQ ID NO: 9).

To explain the above nomenclature, for example, (Gly)₃Lys(Gly)₄ means Gly-Gly-Gly-Lys-Gly-Gly-Gly-Gly. Combinations of Gly and Ala are also preferred.

Non-peptide linkers are also possible. For example, alkyl linkers such as —HN—(CH₂)ₛ—CO—, wherein s=2-20 could be used. These alkyl linkers may further be substituted by any non-sterically hindering group such as lower alkyl (e.g., C₁-C₆), lower acyl, halogen (e.g., Cl, Br), CN, NH₂, phenyl, etc.

Another type of non-peptide linker is a polyethylene glycol group, such as:

—HN—CH₂—CH₂—(O—CH₂—CH₂)ₙ—O—CH₂—CO

wherein n is such that the overall molecular weight of the linker ranges from approximately 101 to 5000, preferably 101 to 500.

In general, it has been discovered that a linker of a length of about 0-14 sub-units (e.g., amino acids) is preferred for the thrombopoietic compounds of the first embodiment of this invention.

The peptide linkers may be altered to form derivatives in the same manner as described above for the TMPs.

The compounds of this first group may further be linear or cyclic. By “cyclic” is meant that at least two separated, i.e., non-contiguous, portions of the molecule are linked to each other. For example, the amino and carboxy terminus of the ends of the molecule could be covalently linked to form a cyclic molecule. Alternatively, the molecule could contain two or more Cys residues (e.g., in the linker), which could cyclize via disulfide bond formation. It is further contemplated that more than one tandem peptide dimer can link to form a dimer of dimers. Thus, for example, a tandem dimer containing a Cys residue can form an intermolecular disulfide bond with a Cys of another such dimer. See, for example, the compound of SEQ ID NO: 20, below.

The compounds of the invention may also be covalently or noncovalently associated with a carrier molecule, such as a linear polymer (e.g., polyethylene glycol, polylysine, dextran, etc.), a branched-chain polymer (see, for example, U.S. Pat. No. 4,289,872 to Denkenwalter et al., issued Sep. 15, 1981; 5,229,490 to Tam, issued Jul. 20, 1993; WO 93/21259 by Frechet et al., published 28 Oct. 1993); a lipid; a cholesterol group (such as a steroid); or a carbohydrate or oligosaccharide. Other possible carriers include one or more water soluble polymer attachments such as polyoxyethylene glycol, or polypropylene glycol as described U.S. Pat. Nos. 4,640,835, 4,496,689, 4,301,144, 4,670,417, 4,791,192 and 4,179,337. Still other useful polymers known in the art include monomethoxy-polyethylene glycol, dextran and dextran derivatives as described in U.S. Pat. No. 5,869,451, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of these polymers.

A preferred such carrier is polyethylene glycol (PEG). The PEG group may be of any convenient molecular weight and may be straight chain or branched. The PEG group may range in molecular weight from about 1 kDa, to about 2 kDa, to about 3 kDa, to about 4 kDa, to about 5 kDa, to about 10 kDa, to about 20 kDa, to about 30 kDa, to about 40 kDa, to about 50 kDa, to about 60 kDa, to about 70 kDa, to about 80 kDa, to about 90 kDa, to about 100 kDa, to about 200 kDa. The average molecular weight of the PEG will preferably range from about 2 kDa to about 100 kDa, more preferably from about 5 kDa to about 50 kDa, most preferably from about 5 kDa to about 10 kDa.

The PEG groups will generally be attached to the compounds of the invention via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group on the target compound (e.g., an aldehyde, amino, ester, thiol, α-haloacetyl, maleimido or hydrazino group).

Carbohydrate (oligosaccharide) groups may conveniently be attached to sites that are known to be glycosylation sites in proteins. Generally, O-linked oligosaccharides are attached to serine (Ser) or threonine (Thr) residues while N-linked oligosaccharides are attached to asparagine (Asn) residues when they are part of the sequence Asn-X-Ser/Thr, where X can be any amino acid except proline. X is preferably one of the 19 naturally occurring amino acids not counting proline. The structures of N-linked and O-linked oligosaccharides and the sugar residues found in each type are different. One type of sugar that is commonly found on both is N-acetylneuraminic acid (referred to as sialic acid). Sialic acid is usually the terminal residue of both N-linked and O-linked oligosaccharides and, by virtue of its negative charge, may confer acidic properties to the glycosylated compound. Such site(s) may be incorporated in the linker of the compounds of this invention and are preferably glycosylated by a cell during recombinant production of the polypeptide compounds (e.g., in mammalian cells such as CHO, BHK, COS). However, such sites may further be glycosylated by synthetic or semi-synthetic procedures known in the art.

Some exemplary peptides of this invention are shown below. Single letter amino acid abbreviations are used, and the linker is shown separated by dashes for clarity. Additional abbreviations: BrAc means bromoacetyl (BrCH₂C(O)) and PEG is polyethylene glycol.

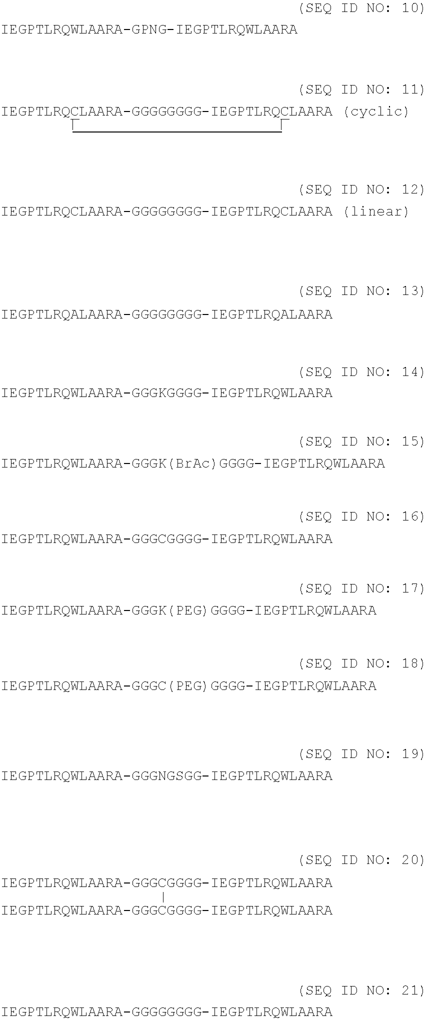

In each of the above compounds, an N-terminal Met (or M residue, using the one-letter code) is contemplated as well. Multimers (e.g., tandem and non-tandem, covalently bonded and non-covalently bonded) of the above compounds are also contemplated.

In a second embodiment of this invention, the compounds described above may further be fused to one or more Fc groups, either directly or through linker groups. In general, the formula of this second group of compounds is:

(Fc)ₘ-(L₂)_{q}-TMP₁-(L₁)ₙ-TMP₂-(L₃)ᵣ-(Fc)ₚ

wherein TMP₁, TMP₂ and n are each as described above; L₁, L₂ and L₃ are linker groups which are each independently selected from the linker groups described above; Fc is an Fc region of an immunoglobulin; m, p, q and r are each independently selected from the group consisting of 0 and 1, wherein at least one of m or p is 1, and further wherein if m is 0 then q is 0, and if p is 0, then r is 0; and physiologically acceptable salts thereof.

Accordingly, the compounds of this second group have structures as defined for the first group of compounds as described above, but these compounds are further fused to at least one Fc group either directly or through one or more linker groups.

The Fc sequence of the above compounds may be selected from the human immunoglobulin IgG-1 heavy chain, see Ellison, J. W. et al., Nucleic Acids Res. 10:4071-4079 (1982), or any other Fc sequence known in the art (e.g. other IgG classes including but not limited to IgG-2, IgG-3 and IgG-4, or other immunoglobulins).

It is well known that Fc regions of antibodies are made up of monomeric polypeptide segments that may be linked into dimeric or multimeric forms by disulfide bonds or by non-covalent association. The number of intermolecular disulfide bonds between monomeric subunits of native Fc molecules ranges from 1 to 4 depending on the class (e.g., IgG, IgA, IgE) or subclass (e.g., IgG1, IgG2, IgG3, IgA1, IgGA2) of antibody involved. The term “Fc” as used herein is generic to the monomeric, dimeric, and multimeric forms of Fc molecules. It should be noted that Fc monomers will spontaneously dimerize when the appropriate Cys residues are present unless particular conditions are present that prevent dimerization through disulfide bond formation. Even if the Cys residues that normally form disulfide bonds in the Fc dimer are removed or replaced by other residues, the monomeric chains will generally dimerize through non-covalent interactions. The term “Fc” herein is used to mean any of these forms: the native monomer, the native dimer (disulfide bond linked), modified dimers (disulfide and/or non-covalently linked), and modified monomers (i.e., derivatives).

Variants, analogs or derivatives of the Fc portion may be constructed by, for example, making various substitutions of residues or sequences.

Variant (or analog) polypeptides include insertion variants, wherein one or more amino acid residues supplement an Fc amino acid sequence. Insertions may be located at either or both termini of the protein, or may be positioned within internal regions of the Fc amino acid sequence. Insertional variants with additional residues at either or both termini can include for example, fusion proteins and proteins including amino acid tags or labels. For example, the Fc molecule may optionally contain an N-terminal Met, especially when the molecule is expressed recombinantly in a bacterial cell such as E. coli.

In Fc deletion variants, one or more amino acid residues in an Fc polypeptide are removed. Deletions can be effected at one or both termini of the Fc polypeptide, or with removal of one or more residues within the Fc amino acid sequence. Deletion variants, therefore, include all fragments of an Fc polypeptide sequence.

In Fc substitution variants, one or more amino acid residues of an Fc polypeptide are removed and replaced with alternative residues. In one aspect, the substitutions are conservative in nature, however, the invention embraces substitutions that ore also non-conservative.

For example, cysteine residues can be deleted or replaced with other amino acids to prevent formation of some or all disulfide crosslinks of the Fc sequences. In particular, the amino acids at positions 7 and 10 of SEQ ID NO: 5 are cysteine residues. One may remove each of these cysteine residues or substitute one or more such cysteine residues with other amino acids, such as Ala or Ser. As another example, modifications may also be made to introduce amino acid substitutions to (1) ablate the Fc receptor binding site; (2) ablate the complement (C1q) binding site; and/or to (3) ablate the antibody dependent cell-mediated cytotoxicity (ADCC) site. Such sites are known in the art, and any known substitutions are within the scope of Fc as used herein. For example, see Molecular Immunology, Vol. 29, No. 5, 633-639 (1992) with regards to ADCC sites in IgG1.

Likewise, one or more tyrosine residues can be replaced by phenylalanine residues as well. In addition, other variant amino acid insertions, deletions (e.g., from 1-25 amino acids) and/or substitutions are also contemplated and are within the scope of the present invention. Conservative amino acid substitutions will generally be preferred. Furthermore, alterations may be in the form of altered amino acids, such as peptidomimetics or D-amino acids.

Fc sequences of the TMP compound may also be derivatized, i.e., bearing modifications other than insertion, deletion, or substitution of amino acid residues. Preferably, the modifications are covalent in nature, and include for example, chemical bonding with polymers, lipids, other organic, and inorganic moieties. Derivatives of the invention may be prepared to increase circulating half-life, or may be designed to improve targeting capacity for the polypeptide to desired cells, tissues, or organs.

It is also possible to use the salvage receptor binding domain of the intact Fc molecule as the Fc part of the inventive compounds, such as described in WO 96/32478, entitled “Altered Polypeptides with Increased Half-Life”. Additional members of the class of molecules designated as Fc herein are those that are described in WO 97/34631, entitled “Immunoglobulin-Like Domains with Increased Half-Lives”. Both of the published PCT applications cited in this paragraph are hereby incorporated by reference.

The Fc fusions may be at the N or C terminus of TMP₁ or TMP₂ or at both the N and C termini of the TMPs. It has been surprisingly discovered that peptides in which an Fc moiety is ligated to the N terminus of the TMP group is more bioactive than the other possibilities, so the fusion having an Fc domain at the N terminus of TMP₁ (i.e., r and p are both 0 and m and q are both 1 in general formula) is preferred. When the Fc chain is fused at the N-terminus of the TMP or linker, such fusion will generally occur at the C-terminus of the Fc chain, and vice versa.

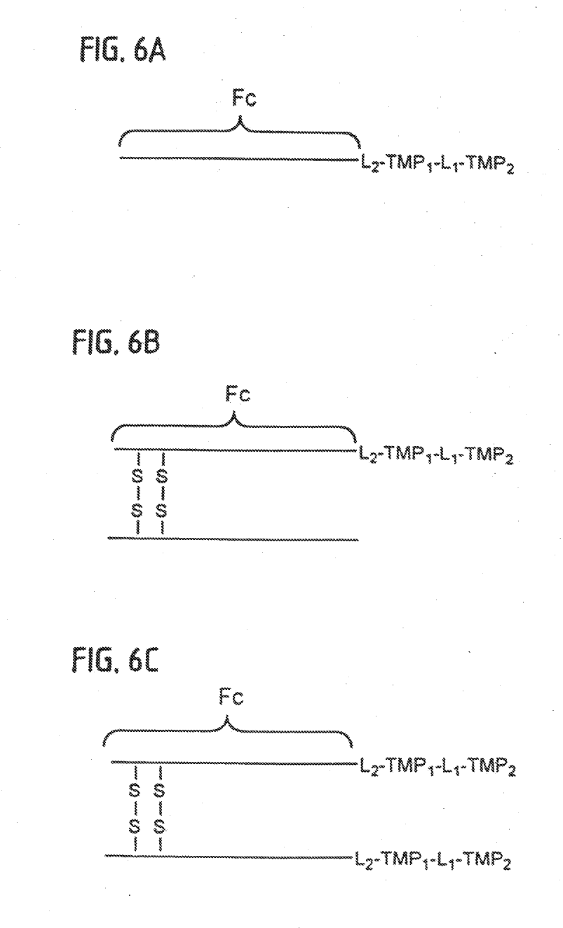

Also preferred are compounds that are dimers (e.g., tandem and non-tandem) of the compounds set forth in the general formula as set out above. In such cases, each Fc chain will be linked to a tandem dimer of TMP peptides. A schematic example of such a compound is shown in FIG. 6 C. A preferred example of this type of compound is based on FIG. 6 C, wherein Fc is a dimer of the compound of SEQ ID NO: 5, each L₂ is (Gly)₅, TMP₁ and TMP₂ are each the compound of SEQ ID NO: 1, and each L₁ is (Gly)₈. This compound is also referred to herein as “Fc-TMP₁-L-TMP₂”. It is also represented as a dimer (through the Fc portion) of SEQ ID NO: 34.

The analogous compound wherein the Fc group is attached through a linker to the C-terminus of the TMP₂ groups in FIG. 6 C is also contemplated and is referred to herein as TMP₁-L-TMP₂-Fc.

Some specific examples of compounds from the second group are provided as follows:

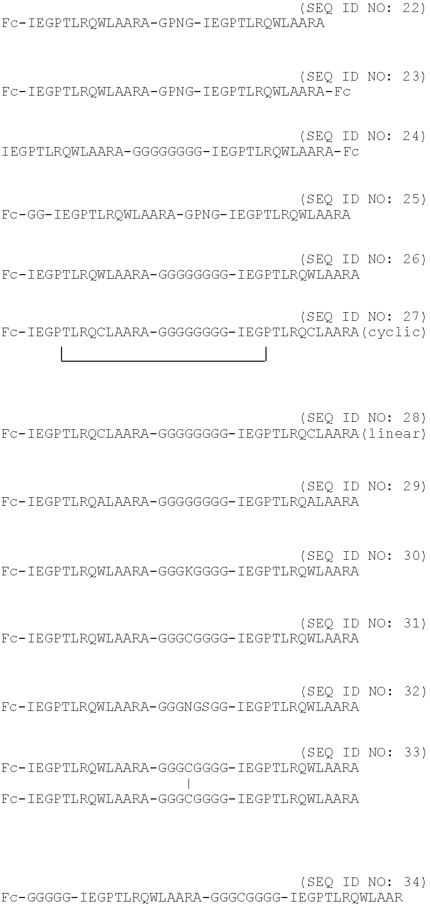

In each of the above compounds, an additional N-terminal Met (or M residue, using the one-letter code) is contemplated as well. The Met residue may be attached at the N-terminus of the Fc group in those cases wherein there is an Fc group attached to the N-terminus of the TMP. In those cases wherein the Fc group is attached at the C-terminus of the TMP, the Met residues could be attached to the N-terminus of the TMP group.

In each of the above cases Fc is preferably the Fc region of the human immunoglobulin IgG1 heavy chain or a biologically active fragment, derivative, or dimer thereof, see Ellison, J. W. et al., Nucleic Acids Res. 10:4071-4079 (1982). The Fc sequence shown in SEQ ID NO: 5 is the most preferred Fc for the above compounds. Also preferred are the above compounds in which the Fc is a dimeric form of the sequence of SEQ ID NO: 5 and each Fc chain is attached to a TMP tandem dimer.

Additionally, although many of the preferred compounds of the second embodiment include one or more tandem dimers in that they possess two linked TMP moieties, other compounds of this invention include tandem multimers of the TMPs, i.e., compounds of the following exemplary structures:

Fc-TMP₁-L-TMP₂-L-TMP₃;

Fc-TMP₁-L-TMP₂-L-TMP₃-L-TMP₄;

Fc-TMP₁-L-TMP₂-L-TMP₃-L-TMP₄-L-TMP₅;

TMP₁-L-TMP₂-L-TMP₃-L-Fc;

TMP₁-L-TMP₂-L-TMP₃-L-TMP₄-L-Fc;

TMP₁-L-TMP₂-L-TMP₃-L-TMP₄-L-TMP₋₅-L-Fc;

wherein TMP₁, TMP₂, TMP₃, TMP₄, and TMP₅ can have the same or different structures, and wherein Fc and each TMP and L is defined as set forth above, and the linkers are each optional. In each case above, the Fc group can be monomeric or dimeric, and in cases where the Fc is dimeric, one or more TMP multimer can be attached to each Fc chains. Also contemplated are other examples wherein the TMP dimers or multimers are attached to both the N and C-termini of one or both Fc chains, including the case wherein TMP dimers or multimers are attached to all four termini of two Fc chains.

In one embodiment, AMP2 is one of the IMP molecules of the invention.

Preferably, the compounds of this second embodiment of the invention will have from about 200 to 400 amino acids in total (i.e., they will be polypeptides).

In another embodiment, the invention provides a core peptide comprising a sequence of amino acids as follows:

X₉-X₈-G-X₁-X₂-X₃-X₄-X₅-X₆-X₇

wherein X₉ is A, C, E, G, I, L, M, P, R, Q, S, T, or V; and X₈ is A, C, D, E, K, L, Q, R, S, T, or V; and X₆ is a b-(2-naphthyl)alanine (referred to herein as “2-Nal”) residue. More preferably, X₉ is A or I; X₈ is D, E, or K; X₁ is C, L, M, P, Q, V; X₂ is F, K, L, N, Q, R, S, T or V; X₃ is C, F, I, L, M, R, S, V or W; X₄ is any of the 20 genetically coded L-amino acids; X₅ is A, D, E, G, K, M, Q, R, S, T, V or Y; and X₇ is C, G, I, K, L, M, N, R or V.

A particularly preferred peptide includes the amino acid sequence: I E G P T L R Q (2-Nal)L A A R A (SEQ ID NO:47), and pegylated forms thereof.

In another embodiment, the peptide compounds of the present invention are preferably dimerized or oligomerized to increase the affinity and/or activity of the compounds. Examples of preferred dimerized peptide compounds include, but are not limited to, the following:

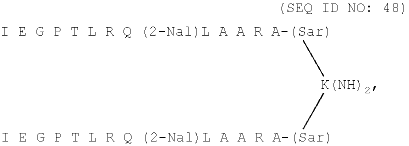

wherein X₁₀ is a sarcosine or β-alanine residue as set out below:

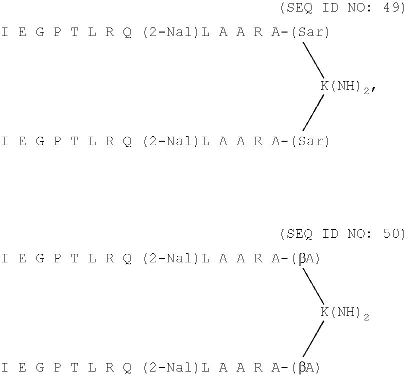

and pegylated forms thereof.

The above structure may also be represented by the following structure:

(H-IEGPTLRQ(2-Nal)LAARX₁₀)₂K—NH₂ (SEQ ID NO: 51)

and pegylated forms thereof.

Additional examples of some preferred dimerized peptide compounds include, but are not limited to, the following:

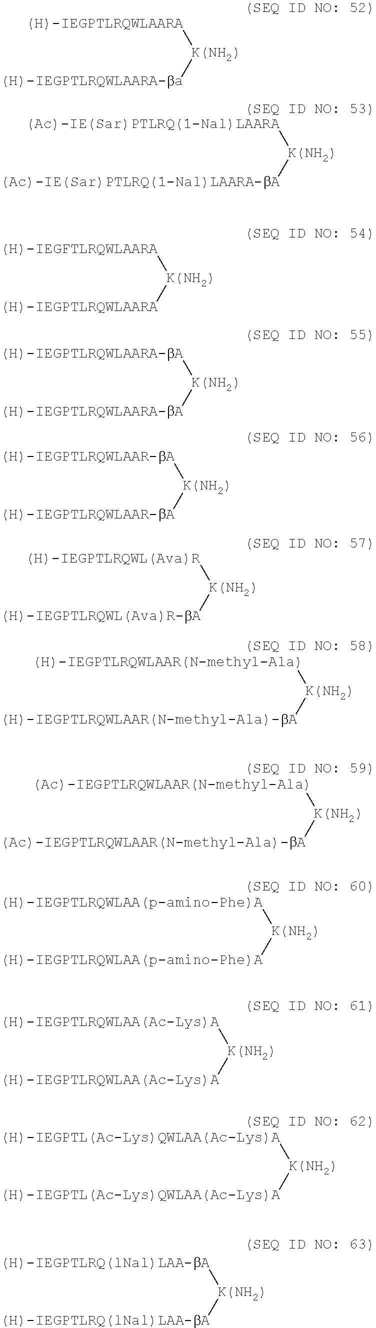

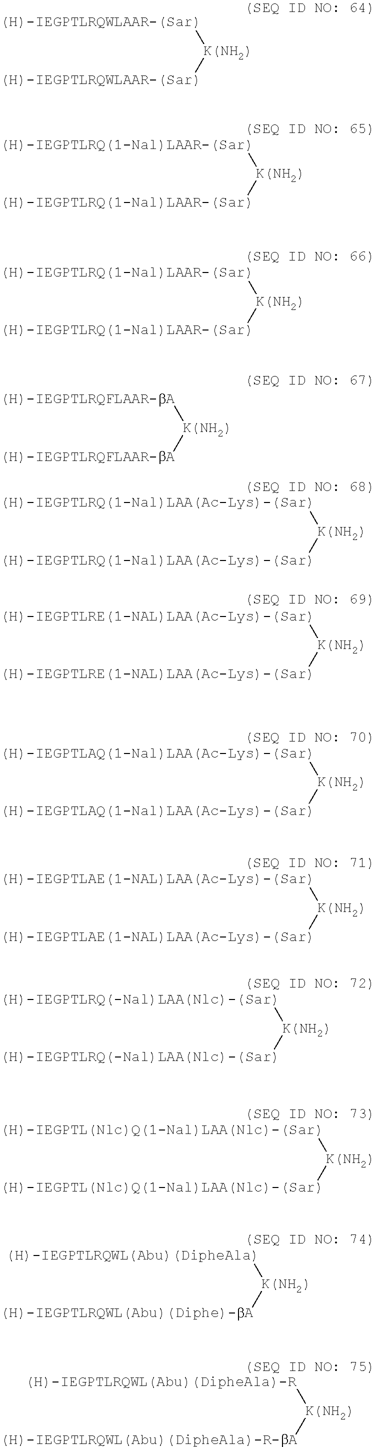

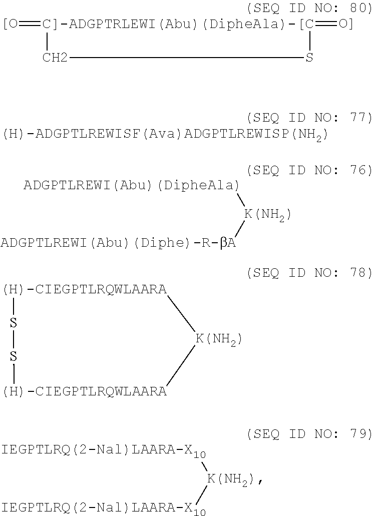

and pegylated forms thereof.

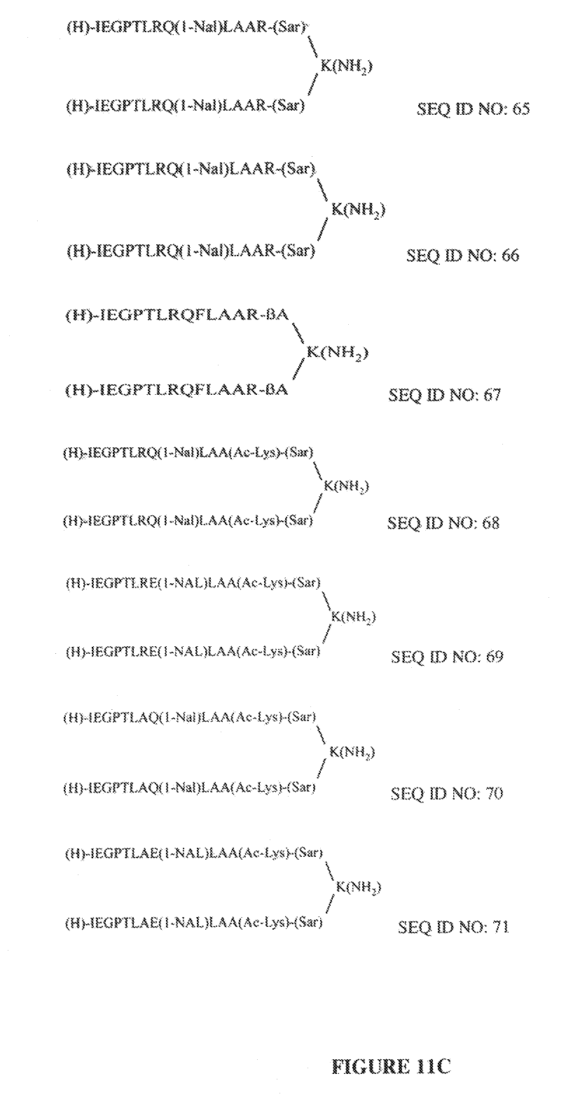

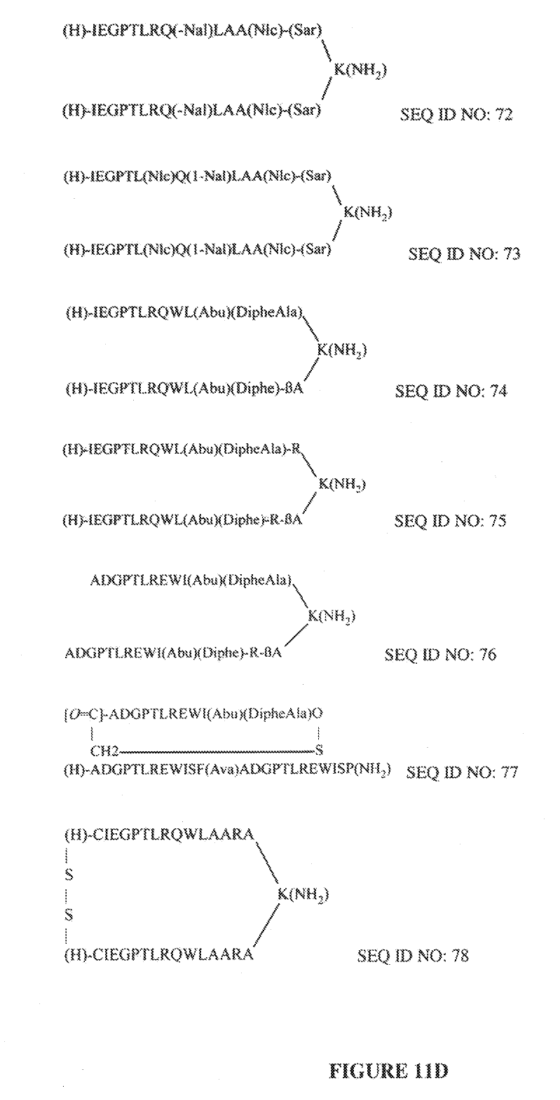

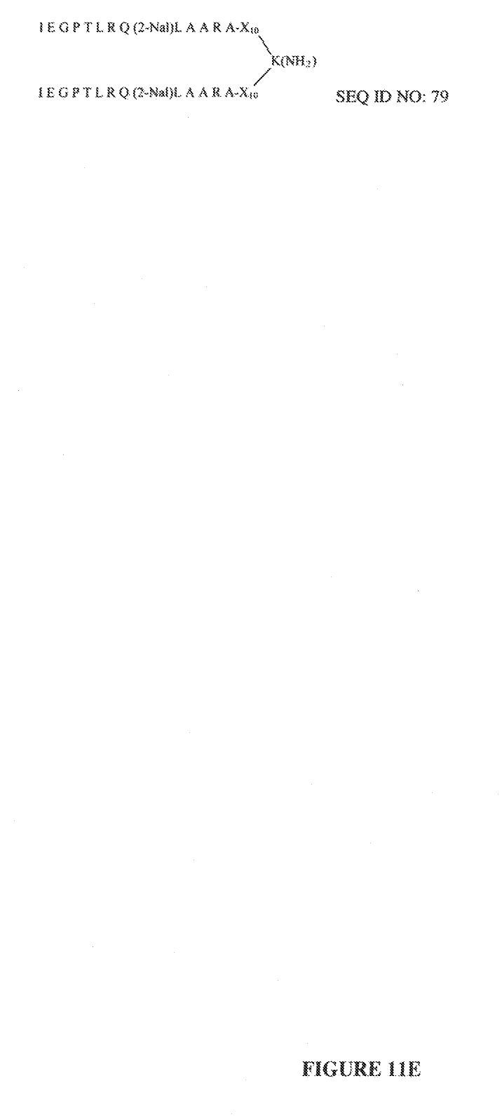

These compounds are also set out in FIG. 11. In addition, these compounds are also provided in International Publication No. WO 2005/023834, U.S. Patent Application Publication Nos. US 2005/0137133, US 2005/0282277, and US 2006/0040866, all incorporated by reference in their entireties herein.

In another embodiment, the invention includes modified antibodies that bind to the mpl receptor and elicit TPO agonisit activity as disclosed in U.S. Patent Application Publication No. US 2004/0091475, incorporated by reference in its entirety herein.

In a further embodiment, the invention includes microproteins comprising mpl binding sequences, which bind to the mpl receptor and elicit TPO agonist activity (i.e. mpl-activating antibodies) as disclosed in International Publication No. WO 2006/094813, incorporated by reference in its entirety herein. In a preferred embodiment, a functional TMP or TPO peptide sequence is grafted into the microprotein.

In still another embodiment, the invention includes TPO mimetic sequences grafted into a human antibody framework. Such biologicially active peptides bind to the mpl receptor and elicit TPO agonist activity as disclosed in International Publication Nos. WO 2004/050017 and WO 02/46238, both incorporated by reference in their entireties herein. For example, the invention includes, but is not limited to, TPO mimetic peptides grafted into the heavy chain CDR3 region of the tetanus toxoid antibody. The invention also contemplates that these molecules may include multiple copies of a TPO mimetic peptide within a single light or heavy chain of an antibody.

In a further embodiment, the invention includes thrombopoietin synthetic antibodies (also known as synthebodies) as disclosed in International Publication No. WO 02/078612, incorporated by reference in its entirety herein. Such TPO synthebodies bind to the c-mpl receptor and demonstrate TPO agonist activity.

### Methods of Making

The compounds of this invention may be made in a variety of ways. Since many of the compounds will be peptides, or will include a peptide, methods for synthesizing peptides are of particular relevance here. For example, solid phase synthesis techniques may be used. Suitable techniques are well known in the art, and include those described in Merrifield, in Chem. Polypeptides, pp. 335-61 (Katsoyannis and Panayotis eds. 1973); Merrifield, J. Am. Chem. Soc. 85:2149 (1963); Davis et al., Biochem. Intl. 10:394-414 (1985); Stewart and Young, Solid Phase Peptide Synthesis (1969); U.S. Pat. No. 3,941,763; Finn et al., The Proteins, 3rd ed., vol. 2, pp. 105-253 (1976); and Erickson et al., The Proteins, 3rd ed., vol. 2, pp. 257-527 (1976). Solid phase synthesis is the preferred technique of making individual peptides since it is the most cost-effective method of making small peptides.

The peptides may also be made in transformed host cells using recombinant DNA techniques. To do so, a recombinant DNA molecule coding for the peptide is prepared. Methods of preparing such DNA and/or RNA molecules are well known in the art. For instance, sequences coding for the peptides could be excised from DNA using suitable restriction enzymes. The relevant sequences can be created using the polymerase chain reaction (PCR) with the inclusion of useful restriction sites for subsequent cloning. Alternatively, the DNA/RNA molecule could be synthesized using chemical synthesis techniques, such as the phosphoramidite method. Also, a combination of these techniques could be used.

The invention also includes a vector encoding the peptides in an appropriate host. The vector comprises the DNA molecule that encodes the peptides operatively linked to appropriate expression control sequences. Methods of effecting this operative linking, either before or after the peptide-encoding DNA molecule is inserted into the vector, are well known. Expression control sequences include promoters, activators, enhancers, operators, ribosomal binding sites, start signals, stop signals, cap signals, polyadenylation signals, and other signals involved with the control of transcription or translation.

The resulting vector comprising the peptide-encoding DNA molecule is used to transform an appropriate host. This transformation may be performed using methods well known in the art.

Any of a large number of available and well-known host cells may be used in the practice of this invention. The selection of a particular host is dependent upon a number of factors recognized by the art. These factors include, for example, compatibility with the chosen expression vector, toxicity to the host cell of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence.

Within these general guidelines, useful microbial hosts include bacteria (such as E. coli), yeast (such as Saccharomyces sp. and Pichia pastoris) and other fungi, insects, plants, mammalian (including human) cells in culture, or other hosts known in the art.

Next, the transformed host is cultured under conventional fermentation conditions so that the desired peptides are expressed. Such fermentation conditions are well known in the art.

Finally, the peptides are purified from the fermentation culture or from the host cells in which they are expressed. These purification methods are also well known in the art.

Compounds that contain derivatized peptides or which contain non-peptide groups may be synthesized by well-known organic chemistry techniques.

### Uses of the Compounds

The compounds of this invention have the ability to bind to and activate the c-Mpl receptor, and/or have the ability to stimulate the production (both in vivo and in vitro) of platelets (“thrombopoietic activity”) and platelet precursors (“megakaryocytopoietic activity”). To measure the activity (-ies) of these compounds, one can utilize standard assays, such as those described in WO95/26746 entitled “Compositions and Methods for Stimulating Megakaryocyte Growth and Differentiation”. In vivo assays are further described in the Examples section herein.

The conditions to be treated by the methods and compositions of the present invention are generally those which involve an existing megakaryocyte/platelet deficiency or an expected or anticipated megakaryocyte/platelet deficiency in the future (e.g., because of planned surgery or platelet donation). Such conditions may be the result of a deficiency (temporary or permanent) of active Mpl ligand in vivo. The generic term for platelet deficiency is thrombocytopenia, and hence the methods and compositions of the present invention are generally available for prophylactically or therapeutically treating thrombocytopenia in patients in need thereof.

The World Health Organization has classified the degree of thrombocytopenia on the number of circulating platelets in the individual (Miller, et al., Cancer 47:210-211 (1981)). For example, an individual showing no signs of thrombocytopenia (Grade 0) will generally have at least 100,000 platelets/mm³. Mild thrombocytopenia (Grade 1) indicates a circulating level of platelets between 79,000 and 99,000/mm³. Moderate thrombocytopenia (Grade 2) shows between 50,000 and 74,000 platelets/mm³ and severe thrombocytopenia is characterized by between 25,000 and 49,000 platelets/mm³. Life-threatening or debilitating thrombocytopenia is characterized by a circulating concentration of platelets of less than 25,000/mm³.

Thrombocytopenia (platelet deficiencies) may be present for various reasons, including chemotherapy and other therapy with a variety of drugs, radiation therapy, surgery, accidental blood loss, and other specific disease conditions. Exemplary specific disease conditions that involve thrombocytopenia and may be treated in accordance with this invention are: aplastic anemia; idiopathic or immune thrombocytopenia (ITP), including idiopathic thrombocytopenic purpura associated with breast cancer; HIV associated ITP and HIV-related thrombotic thrombocytopenic purpura; metastatic tumors which result in thrombocytopenia; systemic lupus erythematosus; including neonatal lupus syndrome splenomegaly; Fanconi's syndrome; vitamin B12 deficiency; folic acid deficiency; May-Hegglin anomaly; Wiskott-Aldrich syndrome; chronic liver disease; myelodysplastic syndrome associated with thrombocytopenia; paroxysmal nocturnal hemoglobinuria; acute profound thrombocytopenia following C7E3 Fab (Abciximab) therapy; alloimmune thrombocytopenia, including maternal alloimmune thrombocytopenia; thrombocytopenia associated with antiphospholipid antibodies and thrombosis; autoimmune thrombocytopenia; drug-induced immune thrombocytopenia, including carboplatin-induced thrombocytopenia, heparin-induced thrombocytopenia; fetal thrombocytopenia; gestational thrombocytopenia; Hughes' syndrome; lupoid thrombocytopenia; accidental and/or massive blood loss; myeloproliferative disorders; thrombocytopenia in patients with malignancies; thrombotic thrombocytopenia purpura, including thrombotic microangiopathy manifesting as thrombotic thrombocytopenic purpura/hemolytic uremic syndrome in cancer patients; autoimmune hemolytic anemia; occult jejunal diverticulum perforation; pure red cell aplasia; autoimmune thrombocytopenia; nephropathia epidemica; rifampicin-associated acute renal failure; Paris-Trousseau thrombocytopenia; neonatal alloimmune thrombocytopenia; paroxysmal nocturnal hemoglobinuria; hematologic changes in stomach cancer; hemolytic uremic syndromes in childhood; hematologic manifestations related to viral infection including hepatitis A virus and CMV-associated thrombocytopenia. Other hepatic diseases or conditions that involve thrombocytopenia and may be treated in accordance with this invention, in addition to viral hepatitis A (HAV) include, but are not limited to, alcoholic hepatitis, autoimmune hepatitis, drug-induced hepatitis, epidemic hepatitis, infectious hepatitis, long-incubation hepatitis, noninfectious hepatitis, serum hepatitis, short-incubation hepatitis, toxic hepatitis, transfusion hepatitis, viral hepatitis B (HBV), viral hepatitis C(HCV), viral hepatitis D (HDV), delta hepatitis, viral hepatitis E (HEV), viral hepatitis F (HFV), viral hepatitis G (HGV), liver disease, inflammation of the liver, hepatic failure, and other hepatic disease. Also, certain treatments for AIDS result in thrombocytopenia (e.g., AZT). Certain wound healing disorders might also benefit from an increase in platelet numbers.

With regard to anticipated platelet deficiencies, e.g., due to future surgery, a compound of the present invention could be administered several days to several hours prior to the need for platelets. With regard to acute situations, e.g., accidental and massive blood loss, a compound of this invention could be administered along with blood or purified platelets.

The compounds of this invention may also be useful in stimulating certain cell types other than megakaryocytes if such cells are found to express Mpl receptor. Conditions associated with such cells that express the Mpl receptor, which are responsive to stimulation by the Mpl ligand, are also within the scope of this invention.

The compounds of this invention may be used in any situation in which production of platelets or platelet precursor cells is desired, or in which stimulation of the c-Mpl receptor is desired. Thus, for example, the compounds of this invention may be used to treat any condition in a mammal wherein there is a need of platelets, megakaryocytes, and the like. Such conditions are described in detail in the following exemplary sources: WO95/26746; WO95/21919; WO95/18858; WO95/21920 and are incorporated herein.

The compounds of this invention may also be useful in maintaining the viability or storage life of platelets and/or megakaryocytes and related cells. Accordingly, it could be useful to include an effective amount of one or more such compounds in a composition containing such cells.

By “mammal” is meant any mammal, including humans, domestic animals including dogs and cats; exotic and/or zoo animals including monkeys; laboratory animals including mice, rats, and guinea pigs; farm animals including horses, cattle, sheep, goats, and pigs; and the like. The preferred mammal is human.

### Pharmaceutical Compositions

The present invention also provides methods of using pharmaceutical compositions of the inventive compounds. Such pharmaceutical compositions may be for administration for injection, or for oral, nasal, transdermal or other forms of administration, including, e.g., by intravenous, intradermal, intramuscular, intramammary, intraperitoneal, intrathecal, intraocular, retrobulbar, intrapulmonary (e.g., aerosolized drugs) or subcutaneous injection (including depot administration for long term release); by sublingual, anal, vaginal, or by surgical implantation, e.g., embedded under the splenic capsule, brain, or in the cornea. The treatment may consist of a single dose or a plurality of doses over a period of time. In general, comprehended by the invention are pharmaceutical compositions comprising effective amounts of a compound of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hyaluronic acid may also be used, and this may have the effect of promoting sustained duration in the circulation. The pharmaceutical compositions optionally may include still other pharmaceutically acceptable liquid, semisolid, or solid diluents that serve as pharmaceutical vehicles, excipients, or media, including but are not limited to, polyoxyethylene sorbitan monolaurate, magnesium stearate, methyl- and propylhydroxybenzoate, starches, sucrose, dextrose, gum acacia, calcium phosphate, mineral oil, cocoa butter, and oil of theobroma. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder, such as lyophilized form Implantable sustained release formulations are also contemplated, as are transdermal formulations.

Contemplated for use herein are oral solid dosage forms, which are described generally in Remington's Pharmaceutical Sciences, 18th Ed. 1990 (Mack Publishing Co. Easton Pa. 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given by Marshall, K., Modern Pharmaceutics, Edited by G. S. Banker and C. T. Rhodes Chapter 10, 1979, herein incorporated by reference. In general, the formulation will include the inventive compound, and inert ingredients which allow for protection against the stomach environment, and release of the biologically active material in the intestine.

Also specifically contemplated are oral dosage forms of the above inventive compounds. If necessary, the compounds may be chemically modified so that oral delivery is efficacious. Generally, the chemical modification contemplated is the attachment of at least one moiety to the compound molecule itself, where said moiety permits (a) inhibition of proteolysis; and (b) uptake into the blood stream from the stomach or intestine. Also desired is the increase in overall stability of the compound and increase in circulation time in the body. Examples of such moieties include: Polyethylene glycol, copolymers of ethylene glycol and propylene glycol, carboxymethyl cellulose, dextran, polyvinyl alcohol, polyvinyl pyrrolidone and polyproline (Abuchowski and Davis, Soluble Polymer-Enzyme Adducts, Enzymes as Drugs, Hocenberg and Roberts, eds., Wiley-Interscience, New York, N.Y., (1981), pp 367-383; Newmark, et al., J. Appl. Biochem. 4:185-189 (1982)). Other polymers that could be used are poly-1,3-dioxolane and poly-1,3,6-tioxocane. Preferred for pharmaceutical usage, as indicated above, are polyethylene glycol moieties.

For the oral delivery dosage forms, it is also possible to use a salt of a modified aliphatic amino acid, such as sodium N-(8-[2-hydroxybenzoyl]amino) caprylate (SNAG), as a carrier to enhance absorption of the therapeutic compounds of this invention. The clinical efficacy of a heparin formulation using SNAG has been demonstrated in a Phase II trial conducted by Emisphere Technologies. See U.S. Pat. No. 5,792,451, “Oral drug delivery composition and methods”.

The therapeutic can be included in the formulation as fine multiparticulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the protein (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An antifrictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment, a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethonium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the protein or derivative either alone or as a mixture in different ratios.

Additives which potentially enhance uptake of the compound are for instance the fatty acids oleic acid, linoleic acid and linolenic acid.

Controlled release formulation may be desirable. The drug could be incorporated into an inert matrix which permits release by either diffusion or leaching mechanisms e.g., gums. Slowly degenerating matrices may also be incorporated into the formulation, e.g., alginates, polysaccharides. Another form of a controlled release of this therapeutic is by a method based on the Oros therapeutic system (Alza Corp.), i.e., the drug is enclosed in a semipermeable membrane which allows water to enter and push drug out through a single small opening due to osmotic effects. Some enteric coatings also have a delayed release effect.

Other coatings may be used for the formulation. These include a variety of sugars which could be applied in a coating pan. The therapeutic agent could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide the optimum film coating. Film coating may be carried out in a pan coater or in a fluidized bed or by compression coating.

Also contemplated herein is pulmonary delivery of the present protein (or derivatives thereof). The protein (or derivative) is delivered to the lungs of a mammal while inhaling and traverses across the lung epithelial lining to the blood stream. (Other reports of this include Adjei et al., Pharmaceutical Research 7:565-569 (1990); Adjei et al., International Journal of Pharmaceutics 63:135-144 (1990)(leuprolide acetate); Braquet et al., Journal of Cardiovascular Pharmacology 13 (suppl.5): s.143-146 (1989)(endothelin-1); Hubbard et al., Annals of Internal Medicine 3:206-212 (1989)(α1-antitrypsin); Smith et al., J. Clin. Invest. 84:1145-1146 (1989)(α1-proteinase); Oswein et al., “Aerosolization of Proteins”, Proceedings of Symposium on Respiratory Drug Delivery II, Keystone, Colo., March, 1990 (recombinant human growth hormone); Debs et al., The Journal of Immunology 140:3482-3488 (1988)(interferon-γ and tumor necrosis factor α) and Platz et al., U.S. Pat. No. 5,284,656 (granulocyte colony stimulating factor).

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the inventive compound. Typically, each formulation is specific to the type of device employed and may involve the use of an appropriate propellant material, in addition to diluents, adjuvants and/or carriers useful in therapy.

The inventive compound should most advantageously be prepared in particulate form with an average particle size of less than 10 μm (or microns), most preferably about 0.5 to 5 μm, for most effective delivery to the distal lung.

Carriers include carbohydrates such as trehalose, mannitol, xylitol, sucrose, lactose, and sorbitol. Other ingredients for use in formulations may include DPPC, DOPE, DSPC and DOPC. Natural or synthetic surfactants may be used. Polyethylene glycol may be used (even apart from its use in derivatizing the protein or analog). Dextrans, such as cyclodextran, may be used. Bile salts and other related enhancers may be used. Cellulose and cellulose derivatives may be used. Amino acids may be used, such as use in a buffer formulation.

Also, the use of liposomes, microcapsules or microspheres, inclusion complexes, or other types of carriers is contemplated.

Formulations suitable for use with a nebulizer, either jet or ultrasonic, will typically comprise the inventive compound dissolved in water at a concentration of about 0.1 to 25 mg of biologically active protein per mL of solution. The formulation may also include a buffer and a simple sugar (e.g., for protein stabilization and regulation of osmotic pressure). The nebulizer formulation may also contain a surfactant, to reduce or prevent surface induced aggregation of the protein caused by atomization of the solution in forming the aerosol.

Formulations for use with a metered-dose inhaler device will generally comprise a finely divided powder containing the inventive compound suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing the inventive compound and may also include a bulking agent, such as lactose, sorbitol, sucrose, mannitol, trehalose, or xylitol in amounts which facilitate dispersal of the powder from the device, e.g., 50 to 90% by weight of the formulation.

Nasal delivery of the inventive compound is also contemplated. Nasal delivery allows the passage of the protein to the blood stream directly after administering the therapeutic product to the nose, without the necessity for deposition of the product in the lung. Formulations for nasal delivery include those with dextran or cyclodextran. Delivery via transport across other mucous membranes is also contemplated.

### Dosages

The dosage regimen involved in a method for treating the above-described conditions will be determined by the attending physician, considering various factors which modify the action of drugs, e.g. the age, condition, body weight, sex and diet of the patient, the severity of any infection, time of administration and other clinical factors. Generally, the dose should be in the range of about 0.1 μg to 100 mg of the inventive compound per kilogram of body weight per day, preferably about 0.1 to 1000 μg/kg; more preferably about 0.1 to 150 μg/kg; yet more preferably about 0.1 to 10 μg/kg; and even more preferably about 3.0 to 10 μg/kg given in daily doses or in equivalent doses at longer or shorter intervals, e.g., every other day, twice weekly, weekly, or twice or three times daily.

The inventive compound may be administered by an initial bolus followed by a continuous infusion to maintain therapeutic circulating levels of drug product. As another example, the inventive compound may be administered as a one-time dose. Those of ordinary skill in the art will readily optimize effective dosages and administration regimens as determined by good medical practice and the clinical condition of the individual patient. The frequency of dosing will depend on the pharmacokinetic parameters of the agents and the route of administration. The optimal pharmaceutical formulation will be determined by one skilled in the art depending upon the route of administration and desired dosage. See for example, Remington's Pharmaceutical Sciences, 18th Ed. (1990, Mack Publishing Co., Easton, Pa. 18042) pages 1435-1712, the disclosure of which is hereby incorporated by reference. Such formulations may influence the physical state, stability, rate of in vivo release, and rate of in vivo clearance of the administered agents. Depending on the route of administration, a suitable dose may be calculated according to body weight, body surface area or organ size. Further refinement of the calculations necessary to determine the appropriate dosage for treatment involving each of the above mentioned formulations is routinely made by those of ordinary skill in the art without undue experimentation, especially in light of the dosage information and assays disclosed herein, as well as the pharmacokinetic data observed in the human clinical trials discussed above. Appropriate dosages may be ascertained through use of established assays for determining blood levels dosages in conjunction with appropriate dose-response data. The final dosage regimen will be determined by the attending physician, considering various factors which modify the action of drugs, e.g. the drug's specific activity, the severity of the damage and the responsiveness of the patient, the age, condition, body weight, sex and diet of the patient, the severity of any infection, time of administration and other clinical factors. As studies are conducted, further information will emerge regarding the appropriate dosage levels and duration of treatment for various diseases and conditions.

The therapeutic methods, compositions and compounds of the present invention may also be employed, alone or in combination with other cytokines, soluble Mpl receptor, hematopoietic factors, interleukins, growth factors or antibodies in the treatment of disease states characterized by other symptoms as well as platelet deficiencies. It is anticipated that the inventive compound will prove useful in treating some forms of thrombocytopenia in combination with general stimulators of hematopoiesis, such as IL-3 or GM-CSF. Other megakaryocytic stimulatory factors, i.e., meg-CSF, stem cell factor (SCF), leukemia inhibitory factor (LIF), oncostatin M (OSM), or other molecules with megakaryocyte stimulating activity may also be employed with Mpl ligand. Additional exemplary cytokines or hematopoietic factors for such co-administration include IL-1 alpha, IL-1 beta, IL-2, IL-3, IL-4, IL-5, IL-6, IL-11, colony stimulating factor-1 (CSF-1), M-CSF, SCF, GM-CSF, granulocyte colony stimulating factor (G-CSF), EPO, interferon-alpha (IFN-alpha), consensus interferon, IFN-beta, IFN-gamma, IL-7, IL-8, IL-9, IL-10, IL-12, IL-13, IL-14, IL-15, IL-16, IL-17, IL-18, thrombopoietin (TPO), angiopoietins, for example Ang-1, Ang-2, Ang-4, Ang-Y, the human angiopoietin-like polypeptide, vascular endothelial growth factor (VEGF), angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil, chemotactic factor 2 α, cytokine-induced neutrophil chemotactic factor 2 β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor α 1, glial cell line-derived neutrophic factor receptor α 2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor receptor, TNF, including TNF0, TNF1, TNF2, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, vascular endothelial growth factor, and chimeric proteins and biologically or immunologically active fragments thereof. It may further be useful to administer, either simultaneously or sequentially, an effective amount of a soluble mammalian Mpl receptor, which appears to have an effect of causing megakaryocytes to fragment into platelets once the megakaryocytes have reached mature form. Thus, administration of an inventive compound (to enhance the number of mature megakaryocytes) followed by administration of the soluble Mpl receptor (to inactivate the ligand and allow the mature megakaryocytes to produce platelets) is expected to be a particularly effective means of stimulating platelet production. The dosage recited above would be adjusted to compensate for such additional components in the therapeutic composition. Progress of the treated patient can be monitored by conventional methods.

In cases where the inventive compounds are added to compositions of platelets and/or megakaryocytes and related cells, the amount to be included will generally be ascertained experimentally by techniques and assays known in the art. An exemplary range of amounts is about 0.1 μg-1 mg inventive compound per 10⁶ cells.

It is understood that the application of the teachings of the present invention to a specific problem or situation will be within the capabilities of one having ordinary skill in the art in light of the teachings contained herein. Examples of the products of the present invention and representative processes for their isolation, use, and manufacture appear below.

### EXAMPLES

The following sets forth exemplary methods for making some of the compounds of the first group disclosed herein.

Materials and Methods

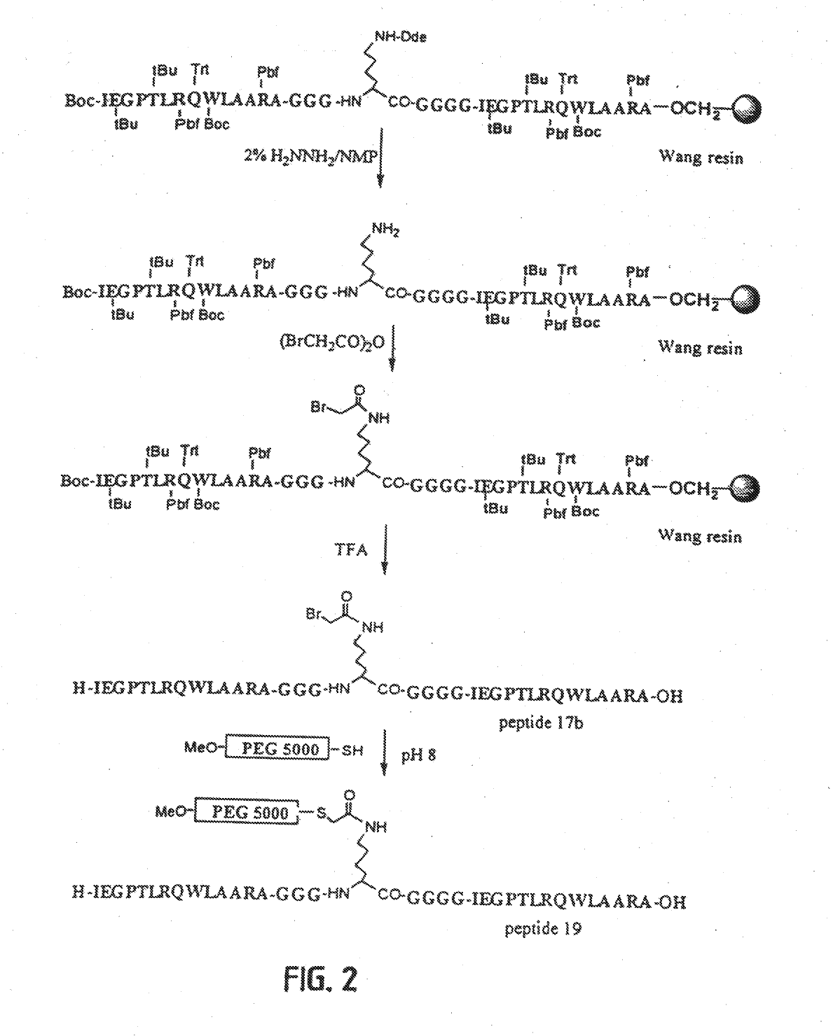

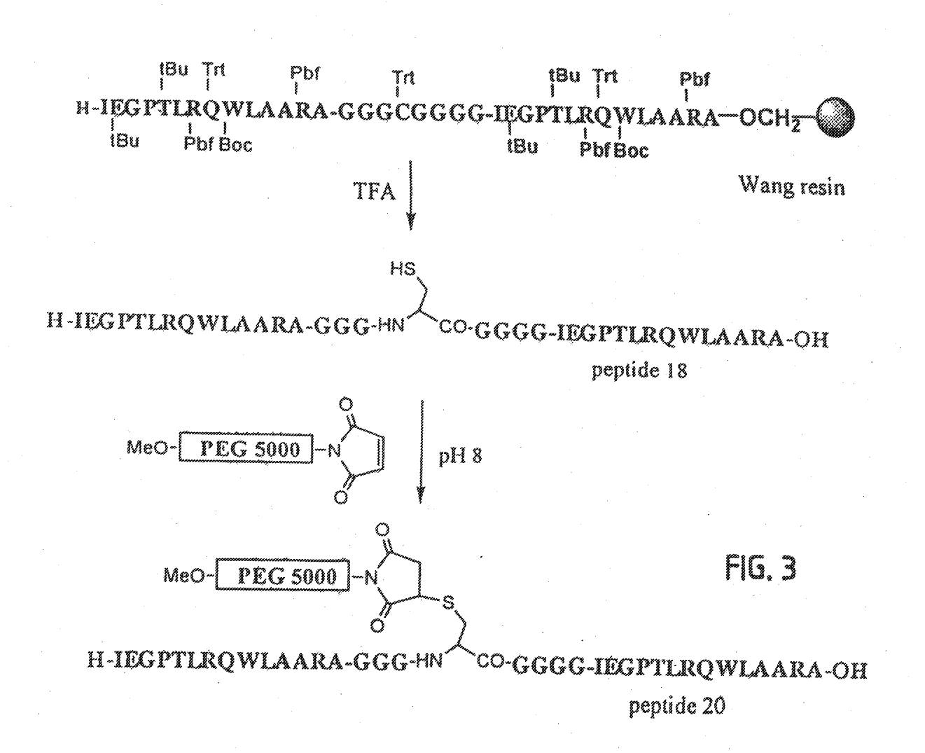

All amino acid derivatives (all of L-configurations) and resins used in peptide synthesis were purchased from Novabiochem. Peptide synthesis reagents (DCC, HOBt, etc.) were purchased in the solution forms from Applied Biosystems, Inc. The two PEG derivatives were from Shearwater Polymers, Inc. All solvents (dichloromethane, N-methylpyrrolidinone, methanol, acetonitrile) were from EM Sciences. Analytical HPLC was run on a Beckman system with a Vydac column (0.46 cm×25 cm, C18 reversed phase, 5 mm), at a flow rate of 1 ml/min and with dual UV detection at 220 and 280 nm. Linear gradients were used for all HPLC operations with two mobile phases: Buffer A—H₂O (0.1% TFA) and Buffer B—acetonitrile (0.1% TFA). The numbered peptides referred to herein, e.g., 17b, 18, 19, and 20, are numbered in reference to Table 1, and some of them are further illustrated in FIGS. 2 and 3.

Peptide synthesis. All peptides were prepared by the well established stepwise solid phase synthesis method. Solid-phase synthesis with Fmoc chemistry was carried out using an ABI Peptide Synthesizer. Typically, peptide synthesis began with a preloaded Wang resin on a 0.1 mmol scale. Fmoc deprotection was carried out with the standard piperidine protocol. The coupling was effected using DCC/HOBt. Side-chain protecting groups were: Glu(O-t-Bu), Thr(t-Bu), Arg(Pbf), Gln(Trt), Trp(t-Boc) and Cys(Trt). For the first peptide precursor for pegylation, Dde was used for side chain protection of the Lys on the linker and Boc-Ile-OH was used for the last coupling. Dde was removed by using anhydrous hydrazine (2% in NMP, 3×2 min), followed by coupling with bromoacetic anhydride preformed by the action of DCC. For peptide 18, the cysteine side chain in the linker was protected by a trityl group. The final deprotection and cleavage of all peptidyl-resins was effected at RT for 4 hr, using trifluoroacetic acid (TFA) containing 2.5% H₂O, 5% phenol, 2.5% triisopropylsilane and 2.5% thioanisole. After removal of TFA, the cleaved peptide was precipitated with cold anhydrous ether. Disulfide formation of the cyclic peptide was performed directly on the crude material by using 15% DMSO in H₂O (pH 7.5). All crude peptides were purified by preparative reverse phase HPLC and the structures were confirmed by ESI-MS and amino acid analysis.

Alternatively, all peptides described above could also be prepared by using the t-Boc chemistry. In this case, the starting resins would be the classic Merrifield or Pam resin, and side chain protecting groups would be: Glu(OBzl), Thr(Bzl), Arg(Tos), Trp(CHO), Cys(p-MeBzl). Hydrogen fluoride (HF) would be used for the final cleavage of the peptidyl resins.

All the tandem dimeric peptides described in this study that have linkers composed of natural amino acids can also be prepared by recombinant DNA technology.

PEGylation. A novel, convergent strategy for the pegylation of synthetic peptides was developed which consists of combining, through forming a conjugate linkage in solution, a peptide and a PEG moiety, each bearing a special functionality that is mutually reactive toward the other. The precursor peptides can be easily prepared with the conventional solid phase synthesis as described above. As described below, these peptides are “preactivated” with an appropriate functional group at a specific site. The precursors are purified and fully characterized prior to reacting with the PEG moiety. Ligation of the peptide with PEG usually takes place in aqueous phase and can be easily monitored by reverse phase analytical HPLC. The pegylated peptides can be easily purified by preparative HPLC and characterized by analytical HPLC, amino acid analysis and laser desorption mass spectrometry.

Preparation of peptide 19. Peptide 17b (12 mg) and MeO-PEG-SH 5000 (30 mg, 2 equiv.) were dissolved in 1 ml aqueous buffer (pH 8). The mixture was incubated at RT for about 30 min and the reaction was checked by analytical HPLC which showed a >80% completion of the reaction. The pegylated material was isolated by preparative HPLC.

Preparation of peptide 20. Peptide 18 (14 mg) and MeO-PEG-maleimide (25 mg) were dissolved in about 1.5 ml aqueous buffer (pH 8). The mixture was incubated at RT for about 30 min, at which time ˜70% transformation was complete as monitored with analytical HPLC by applying an aliquot of sample to the HPLC column. The pegylated material was purified by preparative HPLC.

Bioactivity assay. The TPO in vitro bioassay is a mitogenic assay utilizing an IL-3 dependent clone of murine 32D cells that have been transfected with human mpl receptor. This assay is described in greater detail in WO 95/26746. Cells are maintained in MEM medium containing 10% Fetal Clone II and 1 ng/ml mIL-3. Prior to sample addition, cells are prepared by rinsing twice with growth medium lacking mIL-3. An extended twelve point TPO standard curve is prepared, ranging from 3333 to 39 pg/ml. Four dilutions, estimated to fall within the linear portion of the standard curve, (1000 to 125 pg/ml), are prepared for each sample and run in triplicate. A volume of 100 μl of each dilution of sample or standard is added to appropriate wells of a 96 well microtiter plate containing 10,000 cells/well. After forty-four hours at 37° C. and 10% CO₂, MTS (a tetrazolium compound which is bioreduced by cells to a formazan) is added to each well. Approximately six hours later, the optical density is read on a plate reader at 490 nm. A dose response curve (log TPO concentration vs. O.D.-Background) is generated and linear regression analysis of points which fall in the linear portion of the standard curve is performed. Concentrations of unknown test samples are determined using the resulting linear equation and a correction for the dilution factor.

Abbreviations. HPLC: high performance liquid chromatography; ESI-MS: Electron spray ionization mass spectrometry; MALDI-MS: Matrix-assisted laser desorption ionization mass spectrometry; PEG: Poly(ethylene glycol). All amino acids are represented in the standard three-letter or single-letter codes t-Boc: tert-Butoxycarbonyl; tBu: tert-Butyl; Bzl: Benzyl; DCC: Dicylcohexylcarbodiimide; HOBt: 1-Hydroxybenzotriazole; NMP: N-methyl-2-pyrrolidinone; Pbf: 2,2,4,6,7-pendamethyldihydro-benzofuran-5-sulfonyl; Trt: trityl; Dde: 1-(4,4-dimethyl-2,6-dioxo-cyclohexylidene)ethyl.

### Results

TMP tandem dimers with polyglycine linkers. The design of sequentially linked TMP dimers was based on the assumption that a dimeric form of TMP was required for its effective interaction with c-Mpl (the TPO receptor) and that depending on how they were wound up against each other in the receptor context, the two TMP molecules could be tethered together in the C- to N-terminus configuration in a way that would not perturb the global dimeric conformation. Clearly, the activity of the tandem linked dimers may also depend on proper selection of the length and composition of the linker that joins the C- and -termini of the two sequentially aligned TMP monomers. Since no structural information of the TMP bound to c-Mpl was available, a series of dimeric peptides with linkers composed of 0 to 10 and 14 glycine residues (Table 1) were synthesized. Glycine was chosen because of its simplicity and flexibility. It was reasoned that a flexible polyglycine peptide chain might allow for the free folding of the two tethered TMP repeats into the required conformation, while more sterically hindered amino acid sequences may adopt undesired secondary structures whose rigidity might disrupt the correct packing of the dimeric peptide in the receptor context.

The resulting peptides are readily accessible by conventional solid phase peptide synthesis methods (Merrifiled, R. B., Journal of the American Chemical Society 85:2149 (1963)) with either Fmoc or t-Boc chemistry. Unlike the synthesis of the C-terminally linked parallel dimer (SEQ ID NO: 2) which required the use of an orthogonally protected lysine residue as the initial branch point to build the two peptide chains in a pseudosymmetrical way (Cwirla, S. E. et al., Science 276:1696-1699 (1997)), the synthesis of our tandem dimers was a straightforward, stepwise assembly of the continuous peptide chains from the C— to N-terminus. Since dimerization of TMP had a more dramatic effect on the proliferative activity than binding affinity as shown for the C-terminal dimer (Cwirla, S. E. et al., Science 276:1696-1699 (1997)), the synthetic peptides were tested directly for biological activity in a TPO-dependent cell-proliferation assay using an IL-3 dependent clone of murine 32D cells transfected with the full-length c-Mpl (Palacios, R. et al., Cell 41:727 (1985)). As the test results showed (see Table 1 below), all of the polyglycine linked tandem dimers demonstrated >1000 fold increases in potency as compared to the monomer, and were even more potent than the C-terminal dimer in this cell proliferation assay. The absolute activity of the C-terminal dimer in our assay was lower than that of the native TPO protein, which is different from the previously reported findings in which the C-terminal dimer was found to be as active as the natural ligand (Cwirla, S. E. et al., Science 276:1696-1699 (1997)). This might be due to differences in the conditions used in the two assays. Nevertheless, the difference in activity between tandem dimers© terminal of first monomer linked to N terminal of second monomer) and parallel dimers© terminal of first monomer linked to C terminal of second monomer) in the same assay clearly demonstrated the superiority of tandem dimerized product compared to parallel dimer products. It is interesting to note that a wide range of length is tolerated by the linker. The optimal linker with the selected TMP monomers (SEQ ID NO: 1) apparently is composed of 8 glycines.

Other tandem dimers. Subsequent to this first series of TMP tandem dimers, several other molecules were designed either with different linkers or containing modifications within the monomer itself The first of these molecules, peptide 13, has a linker composed of GPNG, a sequence known to have a high propensity to form a β-turn-type secondary structure. Although still about 100-fold more potent than the monomer, this peptide was found to be >10-fold less active than the GGGG-linked analog. Thus, introduction of a relatively rigid n-turn at the linker region seemed to cause a slight distortion of the optimal agonist conformation in this short linker form.

The Trp9 in the TMP sequence is a highly conserved residue among the active peptides isolated from random peptide libraries. There is also a highly conserved Trp in the consensus sequences of EPO mimetic peptides and this Trp residue was found to be involved in the formation of a hydrophobic core between the two EPO Mimetic Peptides (EMPs) and contributed to hydrophobic interactions with the EPO receptor (Livnah, O. et al., Science 273:464-471 (1996)). By analogy, it was thought that the Trp9 residue in TMP might have a similar function in dimerization of the peptide ligand, and in an attempt to modulate and estimate the effects of noncovalent hydrophobic forces exerted by the two indole rings, several analogs were constructed resulting from mutations at the Trp. So in peptide 14, the Trp residue in each of the two TMP monomers was replaced with a Cys, and an intramolecular disulfide bond was formed between the two cysteines by oxidation which was envisioned to mimic the hydrophobic interactions between the two Trp residues in peptide dimerization. Peptide 15 is the reduced form of peptide 14. In peptide 16, the two Trp residues were replaced by Ala. As the assay data show, all three analogs were inactive. These data further demonstrated that Trp is important for the activity of the TPO mimetic peptide, not just for dimer formation.

The next two peptides (peptide 17a, and 18) each contain in their 8-amino acid linker a Lys or Cys residue. These two compounds are precursors to the two pegylated peptides (peptide 19 and 20) in which the side chain of the Lys or Cys is modified by a polyethylene glycol (PEG) moiety. It was decided to introduce a PEG moiety in the middle of a relatively long linker, so that the large PEG component (5 kDa) is far enough away from the important binding sites in the peptide molecule. PEG is a known biocompatible polymer which is increasingly used as a covalent modifier to improve the pharmacokinetic profiles of peptide- and protein-based therapeutics.

A modular, solution based method was devised for convenient pegylation of synthetic or recombinant peptides. The method is based on the now well established chemoselective ligation strategy which utilizes the specific reaction between a pair of mutually reactive functionalities. So, for pegylated peptide 19, the lysine side chain was preactivated with a bromoacetyl group to give peptide 17b to accommodate reaction with a thiol-derivatized PEG. To do that, an orthogonal protecting group, Dde, was employed for the protection of the lysine ε-amine. Once the whole peptide chain was assembled, the N-terminal amine was reprotected with t-Boc. Dde was then removed to allow for the bromoacetylation. This strategy gave a high quality crude peptide which was easily purified using conventional reverse phase HPLC. Ligation of the peptide with the thiol-modified PEG took place in aqueous buffer at pH 8 and the reaction completed within 30 min. MALDI-MS analysis of the purified, pegylated material revealed a characteristic, bell-shaped spectrum with an increment of 44 Da between the adjacent peaks. For PEG-peptide 20, a cysteine residue was placed in the linker region and its side chain thiol group would serve as an attachment site for a maleimide-containing PEG. Similar conditions were used for the pegylation of this peptide. As the assay data revealed, these two pegylated peptides had even higher in vitro bioactivity as compared to their unpegylated counterparts.

Peptide 21 has in its 8-amino acid linker a potential glycosylation motif, NGS. Since the exemplary tandem dimers are made up of natural amino acids linked by peptide bonds, expression of such a molecule in an appropriate eukaryotic cell system should produce a glycopeptide with the carbohydrate moiety added on the side chain carboxyamide of Asn. Glycosylation is a common post-translational modification process which can have many positive impacts on the biological activity of a given protein by increasing its aqueous solubility and in vivo stability. As the assay data show, incorporation of this glycosylation motif into the linker maintained high bioactivity. The synthetic precursor of the potential glycopeptide had in effect an activity comparable to that of the -(Gly)₈- linked analog. Once glycosylated, this peptide is expected to have the same order of activity as the pegylated peptides, because of the similar chemophysical properties exhibited by a PEG and a carbohydrate moiety.

The last peptide is a dimer of a dimer. It was prepared by oxidizing peptide 18, which formed an intermolecular disulfide bond between the two cysteine residues located at the linker. This peptide was designed to address the possibility that TMP was active as a tetramer. The assay data showed that this peptide was not more active than an average tandem dimer on an adjusted molar basis, which indirectly supports the idea that the active form of TMP is indeed a dimer, otherwise dimerization of a tandem dimer would have a further impact on the bioactivity.

The following Table I summarizes relative activities of the above-described compounds in terms of the EC50 based on in vitro assays as described above.

TABLE I

| Relative | | Po- |
|---|---|---|
| Compound | | tency |
| TPO | | 4.0 |
| TMP monomer (SEQ ID NO: 1) | | 1.0 |
| TMP C-C dimer (SEQ ID NO: 2) | | 3.5 |
| TMP-(Gly)ₙ-TMP: | | |
| 1 | n = 0 | 4.5 |
| 2 | n = 1 | 4.0 |
| 3 | n = 2 | 4.0 |
| 4 | n = 3 | 4.0 |
| 5 | n = 4 | 4.0 |
| 6 | n = 5 | 4.0 |
| 7 | n = 6 | 4.0 |
| 8 | n = 7 | 4.0 |
| 9 | n = 8 | 4.5 |
| 10 | n = 9 | 4.0 |
| 11 | n = 10 | 4.0 |
| 12 | n = 14 | 4.0 |
| 13 | TMP-GPNG-TMP (SEQ ID NO. 10) | 3.0 |
| 14 | 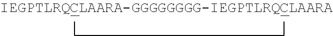 | 0.5 |
| | (SEQ ID NO. 11) | |
| 15 | IEGPTLRQCLAARA-GGGGGGGG-IEGPTLRQCLAARA | 0.5 |
| | (SEQ ID NO. 12) | |
| 16 | IEGPTLRQALAARA-GGGGGGGG-IEGPTLRQALAARA | 0.5 |
| | (SEQ ID NO. 13) | |
| 17a | TMP-GGGKGGGG-TMP (SEQ ID NO. 14) | 4.0 |
| 17b | TMP-GGGK(BrAc)GGGG-TMP (SEQ ID NO. 15) | ND |
| 18 | TMP-GGGCGGGG-TMP (SEQ ID NO. 16) | 4.0 |
| 19 | TMP-GGGK(PEG)GGGG-TMP (SEQ ID NO. 17) | 5.0 |
| 20 | TMP-GGGC(PEG)GGGG-TMP (SEQ ID NO. 18) | 5.0 |
| 21 | TMP-GGGNGSGG-TMP (SEQ ID NO. 19) | 4.0 |
| 22 | 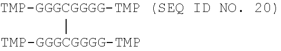 | 4.0 |

NOTE: In Table 1, numerals indicate approximately 1 log of activity, so that the difference in activity between “1” and “4” is approximately 1000-fold. An increment of 0.5 is an intermediate point, so that the difference in activity between “1” and “3.5” is approximately 500-fold. “ND” means not determined.

II. The following sets forth exemplary methods for making some of the compounds of the second group disclosed herein.

### Preparation of an Fc Fusion Compound of the Type Shown in FIG. 6 C.

A DNA sequence coding for the Fc region of human IgG1 fused in-frame to a dimer of the TPO-mimetic peptide (SEQ ID NO: 34) was placed under control of the luxPR promoter in the plasmid expression vector pAMG21 as follows.

The fusion gene was constructed using standard PCR technology. Templates for PCR reactions were the fusion vector containing the Fc sequence

and a synthetic gene encoding the remainder of the compound of SEQ ID NO: 34. The synthetic gene was constructed from the 4 overlapping oligonucleotides shown below:

(SEQ ID NO: 35)
1830-52
AAA GGT GGA GGT GGT GGT ATC GAA GGT CCG
ACT CTG CGT CAG TGG CTG GCT GCT CGT GCT
(SEQ ID NO: 36)
1830-53
ACC TCC ACC ACC AGC ACG AGC AGC CAG
CCA CTG ACG CAG AGT CGG ACC
(SEQ ID NO: 37)
1830-54
GGT GGT GGA GGT GGC GGC GGA GGT ATT GAG GGC
CCA ACC CTT CGC CAA TGG CTT GCA GCA CGC GCA
(SEQ ID NO: 38)
1830-55
AAA AAA AGG ATC CTC GAG ATT ATG CGC GTG CTG
CAA GCC ATT GGC GAA GGG TTG GGC CCT CAA TAC
CTC CGC CGC C

The 4 oligonucleotides were annealed to form the duplex shown below:

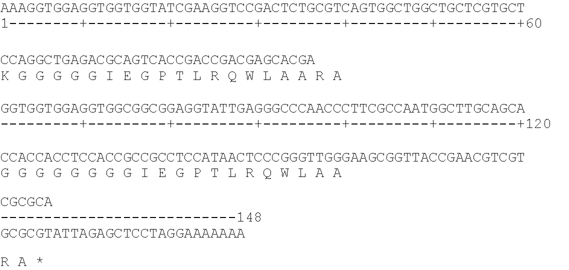

SEQ ID NO: 39 [co-linear oligonucleotides 1830-52 and 1830-54]

SEQ ID NO: 40 [co-linear oligonucleotides 1830-53 and 1830-55] and SEQ ID NO: 41 [the encoded amino acid sequence]

This duplex was amplified in a PCR reaction using 1830-52 and 1830-55 as the sense and antisense primers.

The Fc portion of the molecule was generated in a PCR reaction with Fc DNA using the primers

| (SEQ ID NO: 42) | |
|---|---|
| 1216-52 | AAC ATA AGT ACC TGT AGG ATC G |
| (SEQ ID NO: 43) | |
| 1830-51 | TTCGATACCACCACCTCCACCTTTACCCGGAG- |
| | ACAGGGAGAGGCTCTTCTGC |

The oligonucleotides 1830-51 and 1830-52 contain an overlap of 24 nucleotides, allowing the two genes to be fused together in the correct reading frame by combining the above PCR products in a third reaction using the outside primers, 1216-52 and 1830-55

The final PCR gene product (the full length fusion gene) was digested with restriction endonucleases XbaI and BamHI, and then ligated into the vector pAMG21 (see below), also digested with XbaI and BamHI. Ligated DNA was transformed into competent host cells of E. coli strain 2596 (GM221, described below). Clones were screened for the ability to produce the recombinant protein product and to possess the gene fusion having the correct nucleotide sequence. Protein expression levels were determined from 50 ml shaker flask studies. Whole cell lysates were analyzed for expression of the fusion via Coomassie stained PAGE gels.

The amino acid sequence of the fusion protein is shown below the corresponding nucleotide sequence:

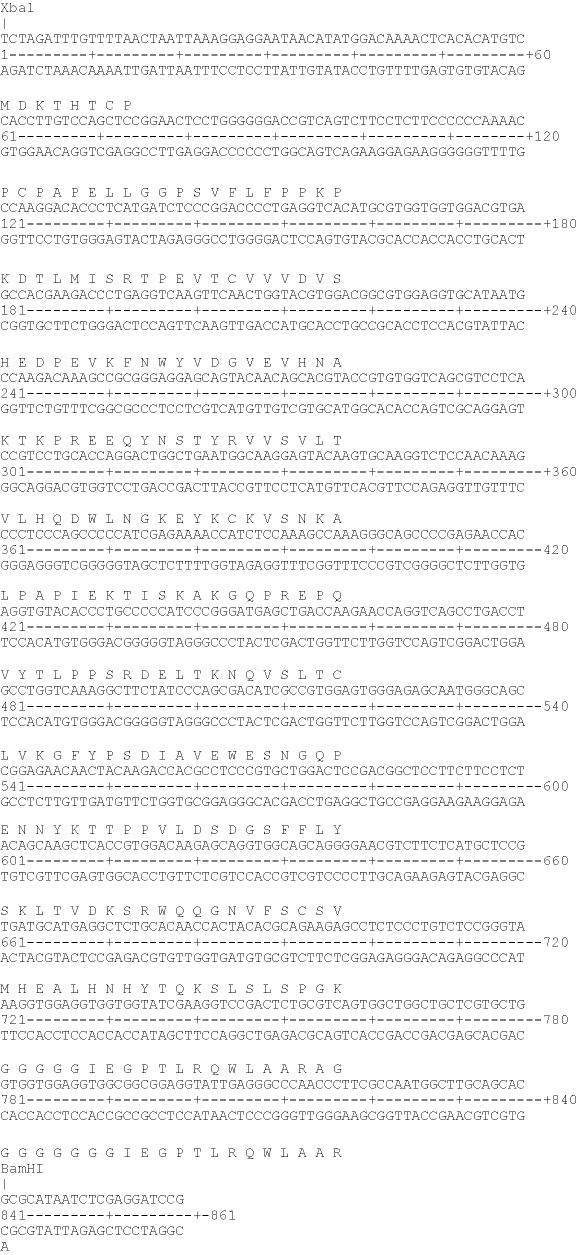

SEQ ID NO: 44 [single strand reading 5′→3′ above],

SEQ ID NO: 45 [single strand reading 3′→5′ above] and

SEQ ID NO: 46 [the encoded amino acid sequence] pAMG21

The expression plasmid pAMG21 is available from the ATCC under accession number 98113, which was deposited on Jul. 24, 1996.

### GM221 (Amgen Host Strain #2596)

The Amgen host strain #2596 is an E. coli K-12 strain that has been modified to contain both the temperature sensitive lambda repressor cI857s7 in the early ebg region and the lacI^{Q} repressor in the late ebg region (68 minutes). The presence of these two repressor genes allows the use of this host with a variety of expression systems, however both of these repressors are irrelevant to the expression from luxP_{R}. The untransformed host has no antibiotic resistances.

The ribosome binding site of the cI857s7 gene has been modified to include an enhanced RBS. It has been inserted into the ebg operon between nucleotide position 1170 and 1411 as numbered in Genbank accession number M64441 Gb_Ba with deletion of the intervening ebg sequence.

The construct was delivered to the chromosome using a recombinant phage called MMebg-cI857s7 enhanced RBS #4 into F′ tet/393. After recombination and resolution only the chromosomal insert described above remains in the cell. It was renamed F′ tet/GM101.

F′ tet/GM101 was then modified by the delivery of a lacI^{Q} construct into the ebg operon between nucleotide position 2493 and 2937 as numbered in the Genbank accession number M64441Gb_Ba with the deletion of the intervening ebg sequence. The construct was delivered to the chromosome using a recombinant phage called AGebg-LacIQ#5 into F′ tet/GM101. After recombination and resolution only the chromosomal insert described above remains in the cell. It was renamed F′ tet/GM221. The F′ tet episome was cured from the strain using acridine orange at a concentration of 25 ug/ml in LB. The cured strain was identified as tetracyline sensitive and was stored as GM221.

The Fc fusion construct contained in plasmid pAMG21 (referred to herein as pAMG21-Fc-TMP-TMP), which in turn is contained in the host strain GM221 has been deposited at the ATCC under accession number 98957, with a deposit date of Oct. 22, 1998.

Expression. Cultures of pAMG21-Fc-TMP-TMP in E. coli GM221 in Luria Broth medium containing 50 μg/ml kanamycin were incubated at 37° C. prior to induction. Induction of Fc-TMP-TMP gene product expression from the luxPR promoter was achieved following the addition of the synthetic autoinducer N-(3-oxohexanoyl)-DL-homoserine lactone to the culture media to a final concentration of 20 ng/ml and cultures were incubated at 37° C. for a further 3 hours. After 3 hours, the bacterial cultures were examined by microscopy for the presence of inclusion bodies and were then collected by centrifugation. Refractile inclusion bodies were observed in induced cultures indicating that the Fc-TMP-TMP was most likely produced in the insoluble fraction in E. coli. Cell pellets were lysed directly by resuspension in Laemmli sample buffer containing 10% β-mercaptoethanol and were analyzed by SDS-PAGE. An intense Coomassie stained band of approximately 30 kDa was observed on an SDS-PAGE gel. The expected gene product would be 269 amino acids in length and have an expected molecular weight of about 29.5 kDa. Fermentation was also carried out under standard batch conditions at the 10 L scale, resulting in similar expression levels of the Fc-TMP-TMP to those obtained at bench scale. Purification of Fc-TMP-TMP.

Cells were broken in water (1/10) by high pressure homogenization (2 passes at 14,000 PSI) and inclusion bodies were harvested by centrifugation (4200 RPM in J-6B for 1 hour). Inclusion bodies were solubilized in 6 M guanidine, 50 mM Tris, 8 mM DTT, pH 8.7 for 1 hour at a 1/10 ratio. The solubilized mixture was diluted 20 times into 2 M urea, 50 mM Tris, 160 mM arginine, 3 mM cysteine, pH 8.5. The mixture was stirred overnight in the cold. At this point in the procedure the Fc-TMP-TMP monomer subunits dimerize to form the disulfide-linked compound having the structure shown in FIG. 6C. and then concentrated about 10 fold by ultafiltration. It was then diluted 3 fold with 10 mM Tris, 1.5 M urea, pH 9. The pH of this mixture was then adjusted to pH 5 with acetic acid. The precipitate was removed by centrifugation and the supernatant was loaded onto a SP-Sepharose Fast Flow column equilibrated in 20 mM NaAc, 100 mM NaCl, pH 5(10 mg/ml protein load, room temperature). The protein was eluted off using a 20 column volume gradient in the same buffer ranging from 100 mM NaCl to 500 mM NaCl. The pool from the column was diluted 3 fold and loaded onto a SP-Sepharose HP column in 20 mM NaAc, 150 mM NaCl, pH 5 (10 mg/ml protein load, room temperature). The protein was eluted off using a 20 column volume gradient in the same buffer ranging from 150 mM NaCl to 400 mM NaCl. The peak was pooled and filtered.

III. The following is a summary of in vivo data in mice with various compounds of this invention.

Mice. Normal female BDF1 approximately 10-12 weeks of age.

Bleed schedule. Ten mice per group treated on day 0, two groups started 4 days apart for a total of 20 mice per group. Five mice bled at each time point, mice were bled a minimum of three times a week. Mice were anesthetized with isoflurane and a total volume of 140-160 μl of blood was obtained by puncture of the orbital sinus. Blood was counted on a Technicon H1E blood analyzer running software for murine blood. Parameters measured were white blood cells, red blood cells, hematocrit, hemoglobin, platelets, neutrophils.

Treatments. Mice were either injected subcutaneously for a bolus treatment or implanted with 7 day micro-osmotic pumps for continuous delivery. Subcutaneous injections were delivered in a volume of 0.2 ml. Osmotic pumps were inserted into a subcutaneous incision made in the skin between the scapulae of anesthetized mice. Compounds were diluted in PBS with 0.1% BSA. All experiments included one control group, labeled “carrier” that were treated with this diluent only. The concentration of the test articles in the pumps was adjusted so that the calibrated flow rate from the pumps gave the treatment levels indicated in the graphs.

Compounds. A dose titration of the compound was delivered to mice in 7 day micro-osmotic pumps. Mice were treated with various compounds at a single dose of 100 ug/kg in 7 day osmotic pumps. Some of the same compounds were then given to mice as a single bolus injection.

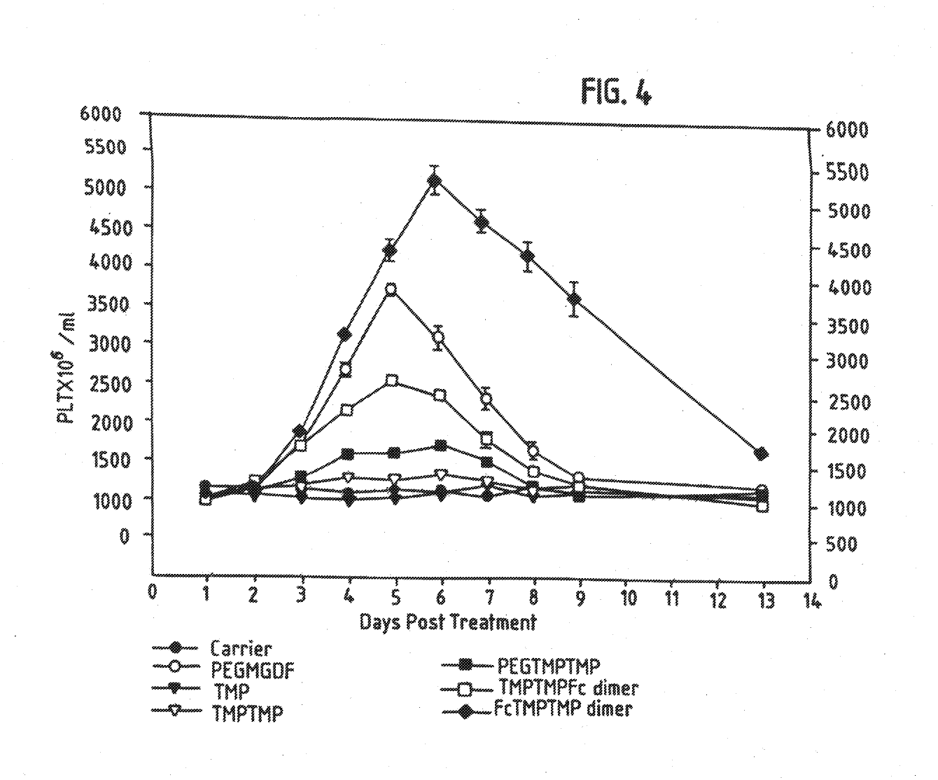

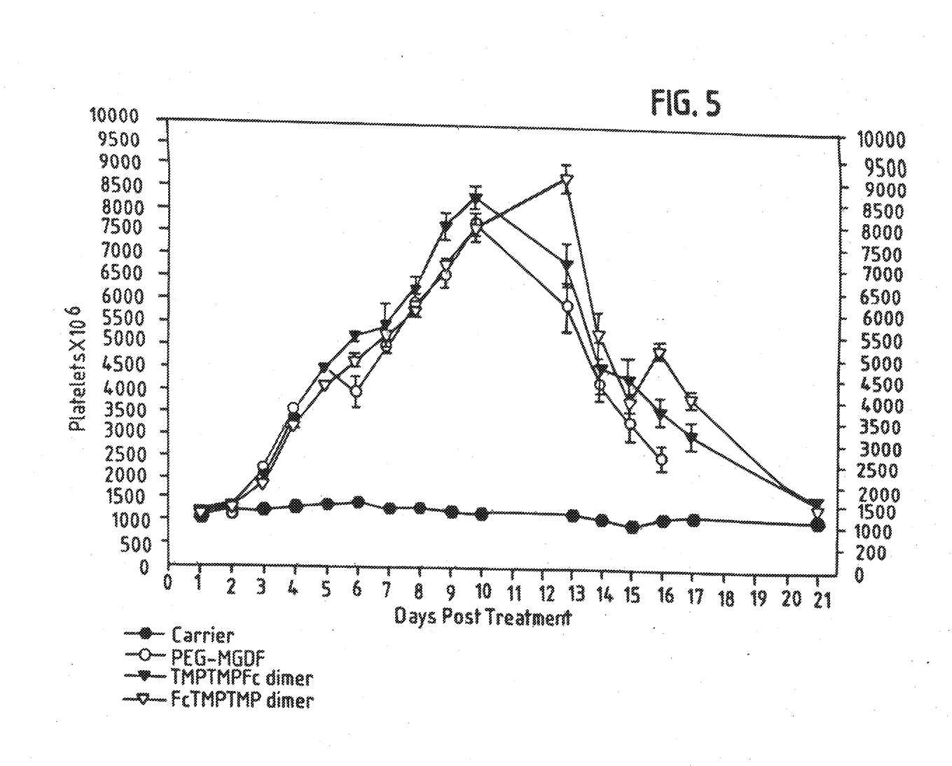

Activity test results. The results of the activity experiments are shown in FIGS. 4 and 5. In dose response assays using 7-day micro-osmotic pumps (data not shown) the maximum effect was seen with the compound of SEQ ID NO: 18 was at 100 μg/kg/day; the 10 μg/kg/day dose was about 50% maximally active and 1 μg/kg/day was the lowest dose at which activity could be seen in this assay system. The compound at 10 μg/kg/day dose was about equally active as 100 μg/kg/day unpegylated rHu-MGDF in the same experiment.

IV. Discussion

It is well accepted that MGDF acts in a way similar to human growth hormone (hGH), i.e., one molecule of the protein ligand binds two molecules of the receptor for its activation (Wells, J. A. et al., Ann. Rev. Biochem. 65:609-634 (1996))). This interaction is mimicked by the action of the much smaller TMP peptide. However, the present studies suggest that this mimicry requires the concerted action of two TMP molecules, as covalent dimerization of TMP in either a C—C parallel or C—N sequential fashion increased the in vitro biological potency of the original monomer by a factor of greater than 10³. The relatively low biopotency of the monomer is probably due to inefficient formation of the noncovalent dimer. A preformed covalent dimer has the ability to eliminate the entropy barrier for the formation of a noncovalent dimer which is exclusively driven by weak, noncovalent interactions between two molecules of the small, 14-residue peptide.

It is interesting to note that most of the tandem dimers are more potent than the C-terminal parallel dimers. Tandem dimerization seems to give the molecule a better fit conformation than does the C—C parallel dimerization. The seemingly unsymmetric feature of a tandem dimer might have brought it closer to the natural ligand which, as an unsymmetric molecule, uses two different sites to bind two identical receptor molecules.

Introduction of the PEG moiety was envisaged to enhance the in vivo activity of the modified peptide by providing it a protection against proteolytic degradation and by slowing down its clearance through renal filtration. It was unexpected that pegylation could further increase the in vitro bioactivity of a tandem dimerized TMP peptide in the cell-based proliferation assay.

V. The following is a summary of in vivo data in monkeys with various compounds of this invention.

In order to evaluate hematological parameters in female rhesus monkeys associated with administration of AMP2 via subcutaneous administration, the following protocol was designed and carried out. Five groups of three monkeys each were assembled. Group 1 served as control and received acetate buffer (20 mM sodium acetate, 0.25 M sodium chloride, pH 5) containing neither AMP2 nor pegylated, recombinant human MGDF (PEG-rHuMGDF). Group 2 received one or more dosage of AMP2 at intervals indicated below; Group 3 received 1000 μg/kg AMP2 at intervals indicated below; Group 4 received 5000 μg/kg AMP2 at intervals indicated below; and Group 5 received 100 μg/kg PEG-rHuMGDF at intervals indicated below.

The day on which the first single dose was administered was designated as Day 0 of Cycle 1. In Cycle 2, doses were administered on Days 21, 23, 25, 28, 30 and 32. During Cycle 3, a single dose was administered on Day 84, and in Cycle 4, a single dose was administration on Day 123. Animals were observed for clinical signs once daily during the acclimation period, three times daily (prior to dosing, immediately to 30 minutes following dosing, and 2 to 3 hours following dosing) on the dosing days, and once daily on the non-dosing days. Food consumption was calculated daily based on the number of food pieces given and the number left over for each animal from 7 days prior to the initiation of the dosing period to the end of the recovery period. Body weight for each animal was measured twice prior to the dosing regimen and twice during the dosing and recovery periods. Blood samples for hematology were prepared once prior to the initiation of dosing and once on Days 1, 3, 5, 7, 9, 11, 13, 15, 20, 22, 24, 26, 29, 31, 33, 35, 37, 39, 41, 43, 45, 47, 49, 55, 62, 69, 76, 83, 85, 87, 89, 91, 93, 95, 97, 99, 101, 103, 105, 111, 122, 124, 126, 128, 130, 132, 134, 136, 138, 140, 142, 144, 150. For pharmacokinetic analysis. 0.5 ml serum samples were collected once prior to dosing and once at 1, 4 and 24 hours after dosing. Samples were collected on Days 0, 21, 32, 84, and 123 and stored at approximately −70° C. until analysis. For antibody analysis, 2 ml blood samples were collected one week prior to the single dose and once on Days 0 (prior to dosing), 6, 13, 20, 27, 34, 41, 48, 55, 62, 69, 76, 83, 90, 97, 104, 111, 118, 129, 136, 143 and 150. Samples were stored at −70° C. until analysis.

Results indicated that platelet values increased in all treated groups with the largest increases seen in the PEG-rHuMGDF and high dose AMP2 groups. In Cycle 1, peak platelet values increased approximately 3.3-fold and 3.1-fold in the PEG-rHuMGDF group (Day 9) and 5000 mg/kg AMP2 group ((Day 9), respectively, compared to the mean platelet count in the control group. The low dose AMP2 platelet values increased approximately 1.5-fold higher than control on the same specified study days. Similar responses were noted in all other cycles.

However, in Cycle 4, the PEG-rHuMGDF group did not demonstrate as large of an increased platelet count as in the previous cycles. The PEG-rHuMGDF group has increased platelet counts of approximately 2-fold that in the control group 9 days after the dose of this cycle. For comparison, the mean platelet count in the highest dose AMP2 group in Cycle 4 was 3.3-fold higher than the control group. Additionally, PEG-rHuMGDF animals has a mean platelet count 53% lower than the control group mean platelet count at the start of Cycle 4 (per dose) and the mean platelet count for the group at the end of Cycle 4*(27 days post dose) was 79% lower than that of the control group. For all AMP2 animals, the mean platelet counts at the start and end of Cycle 4 were ±15% of the platelet count in the control group.

In Cycle 1 and 2, a trend toward a decrease in red blood cell (RBC) counts was noted in all treated groups as compared to control. The decrease was most evident by Days 41 to 43 and the largest decrease in RBC was noted in the PEG-rHuMGDF group. The counts began returning to normal levels (as compared to control) as early as Day 47. The white blood cell (WBC) levels during Cycles 1 and 2 were dramatically increased (2.6-fold) as compared to control on Day 35. A slight increase was noted in the 5000 mg/kg AMP2 group on Day 33. Values headed toward normal (control) levels beginning on Day 37. A similar response was seen in Cycle 3 with no apparent change in WBC in Cycle 4 in any of the treated groups.

During Cycle 3, RBC counts were slightly decreased by Day 13 (following the single Cycle 3 dose) in all treated groups except for the 500 mg/kg AMP2 group. RBC values began returning to normal levels (as compared to control) by Day 17.

In Cycle 4, RBC counts decreased in all treated groups as compared to control except in the 500 mg/kg AMP2 group. Unlike the other cycles, there was more than one nadir present in this cycle. These decreases appeared from Day 1-9 post dose and began to recover as early as Day 11.

The results indicated that an increase in platelet counts, above that of control animals could be detected 7 to 9 days following dosing in all treated animals in all cycles tested. It appeared that the repeated dose phase caused a higher response in platelet production as compared to the single dose phases. By Cycle 4, the platelet response elicited by the PEG-rHuMGDF group was lower compared to the previous cycles and compared to that of the high dose AMP2 response. Decreases in RBC counts were noted in Cycles 1, 2, 3 and 4 in most treated groups at some point during each cycle of the study, however, all hematology parameters returned to normal levels (as compared to control) after dosing cessation.

Overall, these results indicated that treatment with AMP2 was well tolerated in the rhesus monkeys and that AMP2 resulted in increased platelet counts after various cycles of treatment. It did not appear, based on the platelet count results, that there was a biologically significant immune-mediated response to AMP2. In contrast, treatment in the various cycles with PEG-rHuMGDF did show an inhibition in platelet response by Cycle 4, suggesting that antibodies to PEG-rHuMGDF have been generated and these anti-MGDF antibodies may be crossreacting with endogenous rhesus TPO.

VI. The following example describes how patients were recruited for clinical studies using an AMP2 molecule of the invention.

Nine sites in the United States recruited patients with ITP into two sequential trials. The Institutional Review Boards of the participating medical centers approved the protocols, and all patients gave written informed consent before study entry or any screening procedures. Eligibility criteria included: a history of ITP (according to American Society of Hematology guideline criteria 5) of 3 months or more; at least 1 prior treatment for ITP; a mean of two platelet counts during screening <30×10⁹/L (and no count >35×10⁹/L) or <50×10⁹/L (and no count >55×10⁹/L) for patients receiving a constant dose and schedule of corticosteroids; an age of 18 to 65 years; no known risk for thromboembolic events; no history of cardiovascular disease; no active malignancy; and no known history of bone marrow disorder.

Predefined time intervals since the last administration of specified ITP treatments were required before screening for study entry. For example, the intervals were two weeks for IVIG and four months for rituximab. Patients were permitted to enter the study while receiving a constant dose and schedule of corticosteroids and regardless of splenectomy status.

The invention contemplates that such treatment for ITP described herein may be used to treat thrombocytopenia resulting from other diseases, disorders, conditions, or side effects of other medical treatment as well. For example, thrombocytopenia resulting from a hepatitis, such as viral hepatitis A (HAV), alcoholic hepatitis, autoimmune hepatitis, drug-induced hepatitis, epidemic hepatitis, infectious hepatitis, long incubation hepatitis, noninfectious hepatitis, serum hepatitis, short incubation hepatitis, toxic hepatitis, transfusion hepatitis, viral hepatitis B (HBV), viral hepatitis C(HCV), viral hepatitis D (HDV), delta hepatitis, viral hepatitis E (HEV), viral hepatitis F (HFV), viral hepatitis G (HGV), liver disease, inflammation of the liver, hepatic failure, or other hepatic disease may be treated using such compounds as described herein. Also, certain treatments for AIDS which result in thrombocytopenia (e.g., AZT) and certain wound healing disorders might also benefit from treatment using such compounds as described herein.

Likewise, the use of the compounds of this invention may be used in any situation in which production of platelets or platelet precursor cells is desired, or in which stimulation of the c-Mpl receptor is desired. Thus, for example, the compounds of this invention may be used to treat any condition in a mammal wherein there is a need of platelets, megakaryocytes, and the like. Such conditions are described in detail in the following exemplary sources: WO95/26746; WO95/21919; WO95/18858; WO95/21920 and are incorporated herein.

VII. The following example describes a clinical study design using an AMP2 molecule of the invention.

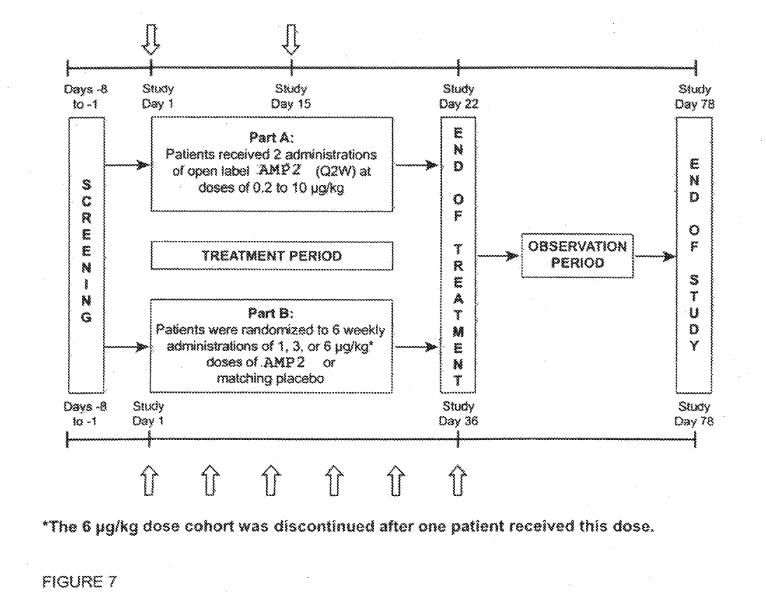

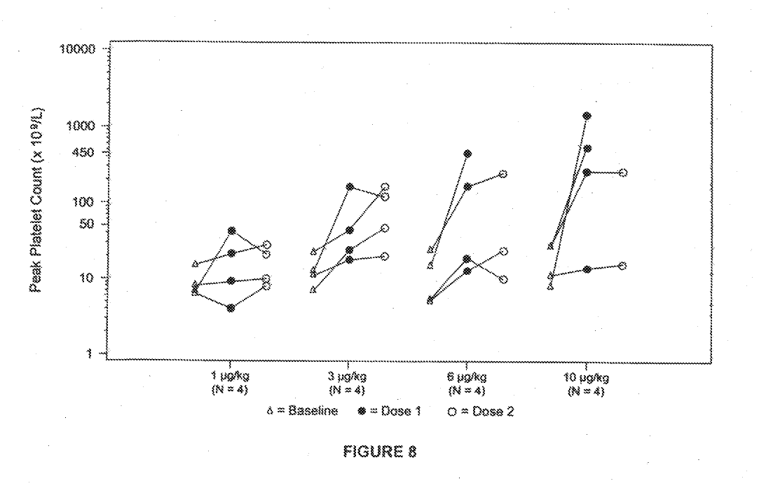

This study of an AMP2 molecule consisted of two parts (see FIG. 7). There was no overlap between patients in Part A and Part B.

Part A was a phase 1, multicenter, open-label, sequential cohort dose escalation trial. The primary objective was to assess the safety, tolerability, and platelet count response of two administrations of an AMP2 molecule in adults with ITP. Secondary objectives were to determine the dose that would elevate platelet counts into a targeted therapeutic window (doubling of baseline and platelet count of 50-450×10⁹/L) and to determine the effect of two exposures to an AMP2 molecule separated by 2-3 weeks.

An AMP2 molecule was administered to cohorts of four patients at doses of 0.2, 0.5, 1, 3, 6, and 10 μg/kg. Drug was administered on study day 1, and patients were observed for 15 days. Health status, complete blood counts, blood chemistries, and anti-AMP2 antibody status were monitored. If the platelet count was =50×10⁹/L on day 15 of the study, a second identical dose was administered. If the platelet count was >50×10⁹/L on day 15, the dose was delayed until day 22; if still >50×10⁹/L, then the second dose was not given. A Data Review Committee, composed of investigators and selected sponsor staff, reviewed data from the ongoing and previous cohorts before making dose escalation decisions. All antibody data from the day 29 samples were reviewed before dosing in the next cohort proceeded. After the treatment period was completed, patients were followed for an 8-week observation period before an end-of-study visit (study day 78).

Part B was a phase 2, multicenter, double-blind, randomized, placebo-controlled, parallel-group trial of three dose levels of an AMP2 molecule. The objectives were to evaluate the safety of an AMP2 molecule in thrombocytopenic patients with ITP and to determine a weekly dose of an AMP2 molecule that elevated platelet counts to a target range (doubling of baseline and platelet count of 50 450×10⁹/L). Eligible patients were randomized to receive one of three dose levels of an AMP2 molecule (1, 3, 6 μg/kg) or placebo (in a 4:1 allocation of an AMP2 molecule to placebo) once weekly for 6 weeks. A protocol amendment later eliminated the 6 μg/kg dose cohort; only one patient was randomized to this cohort. No dose adjustments were allowed, although doses were withheld in the event of excessively high (>350×10⁹/L) platelet counts. Patients were followed for an additional 6 weeks after the last dose of study drug.

In collaboration with the investigators, Amgen designed the study, conducted statistical analyses, and interpreted the data, which Amgen holds. The investigators had unrestricted access to the primary data and were not limited by Amgen.

VIII. The following example describes how TPO and antibody assays were performed.

Blood samples for the assays were drawn at baseline and specified time points under standard clinical conditions. The serum was separated, frozen, and shipped to Amgen for testing. TPO levels were measured using a modification of a commercially available TPO enzyme-linked immunosorbent assay kit (R&D Systems, Minneapolis, Minn.) (Aledort et al., Am. J. Hematol. 76:205-213, 2004). The presence of antibodies that neutralized an AMP2 molecule or TPO was determined using a cell-based bioassay previously described. (Aledort et al., supra; Wang et al., Clin. Pharmacol. Ther. 76:628-638, 2004). The assay is sensitive to approximately 400 ng/ml for the control rabbit-anti-human AMP2 antibody and 200 ng/mL of the control rabbit-antihuman TPO antibody.

IX. The following example provides methods of statistical analysis.

Demographics (age, race, sex) and baseline disease characteristics (see Table II below) were summarized using descriptive statistics. Hematology and other laboratory values and their changes were reported by descriptive statistics. Patients who enrolled in the study, received study drug, completed the study, or withdrew from the study (and the reasons for withdrawal) were summarized by dose cohort, except for the single patient randomized to 6 μg/kg in Part B. Neither part of the study was powered to show a difference between the dose groups, and no statistical hypothesis testing was performed.

TABLE II
DEMOGRAPHICS AND BASELINE CHARACTERISTICS

| | Part A | | | | Part B | | | | | | | |
|---|---|---|---|---|---|---|---|---|---|---|---|---|
| | 2 administrations, | | | | Weekly administration | | | | | | | |
| | 2 weeks apart | | | | for 6 weeks | | | | | | | |
| | AMP2 | | | | AMP2 | | | | | | | |
| | 0.2 to 1 μg/kg | | 3 to 10 μg/kg | | 1 μg/kg | | 3 μg/kg | | 6 μg/kg | | Placebo | |
| | N = 12 | | N = 12 | | N = 8 | | N = 8 | | N = 1 | | N = 4 | |
| Sex - n (%) | | | | | | | | | | | | |
| Female | 8 | (67) | 9 | (75) | 6 | (75) | 5 | (63) | 1 | (100) | 3 | (75) |
| Male | 4 | (33) | 3 | (25) | 2 | (25) | 3 | (38) | 0 | | 1 | (25) |
| Race - n (%) | | | | | | | | | | | | |
| White | 12 | (100) | 10 | (83) | 5 | (63) | 5 | (63) | 1 | (100) | 3 | (75) |
| Black | 0 | | 2 | (17) | 1 | (13) | 0 | | 0 | | 0 | |
| Other | 0 | | 0 | | 2 | (25) | 3 | (38) | 0 | | 1 | (25) |
| Age - yr | | | | | | | | | | | | |
| Median | 45 | | 47 | | 45 | | 53 | | 42 | | 55 | |
| Min, Max | 26, 60 | | 21, 65 | | 20, 63 | | 19, 62 | | — | | 39, 64 | |
| Weight - kg | | | | | | | | | | | | |
| Median | 80 | | 102 | | 73 | | 79 | | 88 | | 87 | |
| Min, Max | 55, 176 | | 57, 135 | | 59, 112 | | 57, 86 | | — | | 68, 110 | |
| Platelets - ×10⁹/L | | | | | | | | | | | | |
| Median | 9 | | 12 | | 17 | | 12 | | 15 | | 29 | |
| Min, Max | 4, 31 | | 5, 27 | | 4.3, 25 | | 5.3, 22.7 | | — | | 6, 49.3 | |
| TPO - pg/mL | | | | | | | | | | | | |
| Median | 75 | | 62 | | 92 | | 105 | | 110 | | 87 | |
| Min, Max | 31, 135 | | 30, 173 | | 68, 185 | | 47, 118 | | — | | 46, 123 | |
| Time Since | | | | | | | | | | | | |
| ITP Diagnosis - yr | | | | | | | | | | | | |
| Median | 7.1 | | 4.7 | | 5.6 | | 9.1 | | 6.4 | | 3.4 | |
| Min, Max | 1.2, 44.4 | | 1.2, 19.5 | | 0.5, 24.9 | | 0.4, 37.0 | | — | | 0.8, 3.7 | |
| Concomitant | | | | | | | | | | | | |
| Corticosteroid Use - n (%) | | | | | | | | | | | | |
| Yes | 2 | (17) | 5 | (42) | 1 | (13) | 3 | (38) | 0 | | 3 | (75) |
| No | 10 | (83) | 7 | (58) | 7 | (88) | 5 | (63) | 1 | (100) | 1 | (25) |
| Splenectomy Status - n (%) | | | | | | | | | | | | |
| Yes | 11 | (92) | 8 | (67) | 5 | (63) | 7 | (88) | 1 | (100) | 1 | (25) |

| | | | | | | | | | | | |
|---|---|---|---|---|---|---|---|---|---|---|---|
| No | 1 | (8) | 4 | (33) | 3 | (38) | 1 | (13) | 0 | 3 | (75) |

In each part of the study, a general linear model was used to investigate the relationship between peak platelet count and baseline TPO level, adjusted for the AMP2 molecule dose received. Logistic regression models, adjusted for the AMP2 molecule dose, were used to fit platelet response status (doubling of baseline and platelet count >50×10⁹/L) as an outcome with each of the following variables as a predictor: baseline serum TPO concentrations, baseline platelet count, splenectomy status, and concurrent ITP therapy.

Platelet counts measured after the administration of rescue medications were excluded from the efficacy analysis. A rescue medication was defined as: 1) concomitant medication indicated for increasing platelet counts not being administered at baseline, or 2) any increase in the dose or frequency of corticosteroids administered at baseline.

X. The following example describes Study Part A.

### Study Population

Twenty-four ITP patients were enrolled into six dose cohorts (four each at 0.2, 0.5, 1, 3, 6, and 10 μg/kg). Table II summarizes baseline demographic information and hematology variables for the 0.2 to 1 μg/kg and the 3 to 10 μg/kg cohorts. Patients were predominantly female (n=17) and white (n=22), with a median age of 45 years and a median weight of 86 kg. The median platelet count at baseline was 11×10⁹/L, and the median time since the diagnosis of ITP was 6.2 years. Seven patients (29%) were using concomitant corticosteroids at baseline, and 19 (79%) had undergone a splenectomy.

### Efficacy

With the exception of one patient in the 0.2 μg/kg dose cohort, who had received rituximab 4 weeks previously, patients in the 0.2, 0.5, and 1 μg/kg cohorts did not achieve the target platelet response (doubling of baseline and platelet count of 50-450×10⁹/L). However, in the 3, 6, and 10 μg/kg cohorts, 4 of 12 patients (33%) achieved the target response. The median time to achieve the target response was 8 days in the 3 μg/kg cohort, 6.5 days in the 6 μg/kg cohort, and 7 days in the 10 μg/kg cohort, with an overall range of 5 to 8 days. Three additional patients in the 3, 6, and 10 μg/kg cohorts had platelet count increases to >450×10⁹/L, for a total of 7 of 12 patients (58%) with a peak platelet count >50×10⁹/L (FIG. 8). The increase in platelet counts appeared to be dose-dependent. Mean peak platelet counts after the first injection of an AMP2 molecule were 163×10⁹/L in the 3 μg/kg cohort, 309×10⁹/L in the 6 μg/kg cohort, and 746×10⁹/L in the 10 μg/kg cohort. Median times to reach peak platelet counts were 11, 10, and 14 days, respectively. Platelet count responses were highly variable.

No statistically significant relationship was observed between peak platelet count and baseline TPO level. Of the baseline factors analyzed in the logistic regression models, only platelet count in part A was predictive of platelet response (the higher the baseline platelet count, the greater likelihood of a platelet response, p=0.0491).

### Safety

Since doses less than 3 μg/kg did not demonstrate a biological effect on the platelet count, the adverse event data were considered in two groups: those in dose cohorts 0.2 to 1 μg/kg and those in dose cohorts 3 to 10 μg/kg (see Table III). The AMP2 molecule was generally well tolerated at the doses tested. The most frequently reported adverse events were contusion/ecchymosis in 6 of 12 patients in the 0.2 to 1 μg/kg cohorts and 10 of 12 patients in the 3 to 10 μg/kg cohorts (overall 16/24, 67%) and mild to moderate headache in 6 of 12 patients in the 0.2 to 1 μg/kg cohorts and 5 of 12 patients in the 3 to 10 μg/kg cohorts (overall 11/24, 46%).

Serious adverse events were reported for 3 patients (see Table III). One patient in the 0.2 μg/kg cohort experienced grade 3 vertigo, reported as unrelated to the AMP2 molecule, and was briefly hospitalized. Another patient in the 0.2 μg/kg cohort experienced a life-threatening cerebral hemorrhage, reported as unrelated to the AMP2 molecule. One patient in the 10 μg/kg cohort developed a transient platelet count decrease to <10×10⁹/L after discontinuation of treatment, reported as related to the AMP2 molecule.

No patients in this study tested positive for anti-AMP2 or anti-TPO antibodies. With the exception of platelet counts, no clinically significant changes were observed in hematologic or serum chemistry laboratory values.

TABLE III
SUMMARY OF ADVERSE EVENTS

| | Part A | | | | Part B | | | | | |
|---|---|---|---|---|---|---|---|---|---|---|
| | 2 administrations, | | | | Weekly administration | | | | Part A + | |
| | 2 weeks apart | | | | for 6 weeks | | | | Part B | |
| | AMP2 | | | | AMP2 | | | | AMP2 | |
| | 0.2 to 1 μg/kg | | 3 to 10 μg/kg | | 1 to 6 μg/kg | | Placebo | | All Doses | |
| | N = 12 | | N = 12 | | N = 17 | | N = 4 | | N = 41 | |
| Adverse Event | n (%) | | n (%) | | n (%) | | n (%) | | n (%) | |
| Contusion and/or ecchymosis | 6 | (50) | 10 | (83) | 10 | (59) | 3 | (75) | 26 | (63) |
| Headache | 6 | (50) | 5 | (42) | 5 | (29) | 0 | | 16 | (39) |
| Petechiae | 3 | (25) | 5 | (42) | 4 | (24) | 1 | (25) | 12 | (29) |
| Epistaxis | 1 | (8) | 2 | (17) | 7 | (41) | 2 | (50) | 10 | (24) |
| Fatigue | 5 | (42) | 3 | (25) | 1 | (6) | 0 | | 9 | (22) |
| Oral mucosal blistering | 1 | (8) | 2 | (17) | 5 | (29) | 0 | | 8 | (20) |
| Gingival bleeding | 1 | (8) | 2 | (17) | 4 | (24) | 1 | (25) | 7 | (17) |
| Dizziness | 1 | (8) | 3 | (25) | 2 | (12) | 1 | (25) | 6 | (15) |

| Upper respiratory tract infection | 4 | (33) | 2 | (17) | 0 | | 0 | 6 | (15) |
|---|---|---|---|---|---|---|---|---|---|
| NOS | | | | | | | | | |
| Excoriation | 1 | (8) | 1 | (8) | 3 | (18) | 0 | 5 | (12) |

| | | | | | | | | | |
|---|---|---|---|---|---|---|---|---|---|
| Nausea | 3 | (25) | 0 | 2 | (12) | 1 | (25) | 5 | (12) |

| | | | | | | | | | |
|---|---|---|---|---|---|---|---|---|---|
| Arthralgia | 3 | (25) | 1 | (8) | 0 | 1 | (25) | 4 | (10) |

| | | | | | | | | | |
|---|---|---|---|---|---|---|---|---|---|
| Edema peripheral | 1 | (8) | 2 | (17) | 1 | (6) | 0 | 4 | (10) |

| | | | | | | | | |
|---|---|---|---|---|---|---|---|---|
| Rash NOS | 3 | (25) | 1 | (8) | 0 | 0 | 4 | (10) |

| Thrombocytopenia | 0 | 1 | (8) | 3 | (18) | 0 | | 4 | (10) |
|---|---|---|---|---|---|---|---|---|---|
| Back pain | 0 | 2 | (17) | 1 | (6) | 2 | (50) | 3 | (7) |

| | | | | | | | | |
|---|---|---|---|---|---|---|---|---|
| Diarrhea | 0 | 0 | 3 | (18) | 1 | (25) | 3 | (7) |

| | | | | | | | | | |
|---|---|---|---|---|---|---|---|---|---|
| Dyspnea | 1 | (8) | 1 | (8) | 1 | (6) | 0 | 3 | (7) |

| | | | | | | | | |
|---|---|---|---|---|---|---|---|---|
| Musculoskeletal stiffness | 2 | (17) | 0 | 1 | (6) | 0 | 3 | (7) |
| Pain in extremity | 2 | (17) | 0 | 1 | (6) | 0 | 3 | (7) |

| | | | | | | | | |
|---|---|---|---|---|---|---|---|---|
| Pharyngolaryngeal pain | 0 | 2 | (17) | 1 | (6) | 0 | 3 | (7) |

| | | | | | | | |
|---|---|---|---|---|---|---|---|
| Purpura | 0 | 0 | 3 | (18) | 0 | 3 | (7) |

| | | | | | | | | |
|---|---|---|---|---|---|---|---|---|
| Purpura NOS | 2 | (17) | 1 | (8) | 0 | 0 | 3 | (7) |

| | | | | | | | | | |
|---|---|---|---|---|---|---|---|---|---|
| Stomatitis | 0 | 1 | (8) | 2 | (12) | 2 | (50) | 3 | (7) |

| | | | | | | | | |
|---|---|---|---|---|---|---|---|---|
| Upper respiratory tract infection | 0 | 0 | 3 | (18) | 1 | (25) | 3 | (7) |

| | | | | | | | | |
|---|---|---|---|---|---|---|---|---|
| Venipuncture site bruise | 1 | (8) | 0 | 2 | (12) | 0 | 3 | (7) |

| | | | | | | | | | |
|---|---|---|---|---|---|---|---|---|---|
| Vaginal hemorrhage* | 0 | 1 | (11) | 1 | (8) | 1 | (33) | 2 | (7) |

| | | | | | | | | |
|---|---|---|---|---|---|---|---|---|
| Abdominal pain | 0 | 0 | 2 | (12) | 1 | (25) | 2 | (5) |

| | | | | | | | | |
|---|---|---|---|---|---|---|---|---|
| Anxiety | 0 | 1 | (8) | 1 | (6) | 0 | 2 | (5) |
| Constipation | 0 | 1 | (8) | 1 | (6) | 0 | 2 | (5) |

| | | | | | | | | |
|---|---|---|---|---|---|---|---|---|
| Cough | 1 | (8) | 1 | (8) | 0 | 0 | 2 | (5) |
| Dyspepsia | 1 | (8) | 1 | (8) | 0 | 0 | 2 | (5) |

| | | | | | | | |
|---|---|---|---|---|---|---|---|
| Erythema | 0 | 0 | 2 | (12) | 0 | 2 | (5) |
| Flushing | 0 | 0 | 2 | (12) | 0 | 2 | (5) |
| Hematochezia | 0 | 0 | 2 | (12) | 0 | 2 | (5) |
| Herpes simplex | 0 | 0 | 2 | (12) | 0 | 2 | (5) |

| | | | | | | | | | |
|---|---|---|---|---|---|---|---|---|---|
| Injection site bruising | 1 | (8) | 1 | (8) | 0 | 1 | (25) | 2 | (5) |

| | | | | | | | |
|---|---|---|---|---|---|---|---|
| Insomnia | 0 | 0 | 2 | (12) | 0 | 2 | (5) |

| | | | | | | | | |
|---|---|---|---|---|---|---|---|---|
| Joint stiffness | 1 | (8) | 1 | (8) | 0 | 0 | 2 | (5) |
| Mouth ulceration | 1 | (8) | 1 | (8) | 0 | 0 | 2 | (5) |

| | | | | | | | |
|---|---|---|---|---|---|---|---|
| Muscle cramp | 0 | 0 | 2 | (12) | 0 | 2 | (5) |

| | | | | | | | |
|---|---|---|---|---|---|---|---|
| Pain NOS | 2 | (17) | 0 | 0 | 0 | 2 | (5) |

| | | | | | | | |
|---|---|---|---|---|---|---|---|
| Rash erythematous | 0 | 0 | 2 | (12) | 0 | 2 | (5) |

| Sinusitis NOS | 0 | | 2 | (17) | 0 | 0 | 2 | (5) |
|---|---|---|---|---|---|---|---|---|
| Tooth abscess | 1 | (8) | 1 | (8) | 0 | 0 | 2 | (5) |

| | | | | | | | | | |
|---|---|---|---|---|---|---|---|---|---|
| Toothache | 0 | 1 | (8) | 1 | (6) | 1 | (25) | 2 | (5) |

| | | | | | | | |
|---|---|---|---|---|---|---|---|
| Dysmenorrhea* | 0 | 0 | 1 | (8) | 0 | 1 | (3) |
| Menorrhagia* | 0 | 0 | 1 | (8) | 0 | 1 | (3) |

| | | | | | | | |
|---|---|---|---|---|---|---|---|
| Abdominal discomfort | 0 | 1 | (8) | 0 | 0 | 1 | (2) |

| | | | | | | | | |
|---|---|---|---|---|---|---|---|---|
| Abdominal pain upper | 1 | (8) | 0 | 0 | 1 | (25) | 1 | (2) |

| | | | | | | | |
|---|---|---|---|---|---|---|---|
| Anemia | 0 | 0 | 1 | (6) | 0 | 1 | (2) |

| | | | | | | | |
|---|---|---|---|---|---|---|---|
| Aphthous stomatitis | 0 | 1 | (8) | 0 | 0 | 1 | (2) |
| Asthenia | 0 | 1 | (8) | 0 | 0 | 1 | (2) |

| | | | | | | | |
|---|---|---|---|---|---|---|---|
| Blood pressure increased | 0 | 0 | 1 | (6) | 0 | 1 | (2) |

| | | | | | | | |
|---|---|---|---|---|---|---|---|
| Cerebral hemorrhage | 1 | (8) | 0 | 0 | 0 | 1 | (2) |

| | | | | | | | |
|---|---|---|---|---|---|---|---|
| Cognitive disorder | 0 | 0 | 1 | (6) | 0 | 1 | (2) |
| Decreased appetite | 0 | 0 | 1 | (6) | 0 | 1 | (2) |
| Depressed mood | 0 | 0 | 1 | (6) | 0 | 1 | (2) |
| Depression | 0 | 0 | 1 | (6) | 0 | 1 | (2) |

| | | | | | | | |
|---|---|---|---|---|---|---|---|
| Diarrhea NOS | 1 | (8) | 0 | 0 | 0 | 1 | (2) |

| | | | | | | | |
|---|---|---|---|---|---|---|---|
| Ear hemorrhage | 0 | 1 | (8) | 0 | 0 | 1 | (2) |

| | | | | | | | |
|---|---|---|---|---|---|---|---|
| Ejaculation disorder NOS* | 1 | (25) | 0 | 0 | 0 | 1 | (8) |

| | | | | | | | |
|---|---|---|---|---|---|---|---|
| Esophageal spasm | 0 | 0 | 1 | (6) | 0 | 1 | (2) |

| | | | | | | | |
|---|---|---|---|---|---|---|---|
| Eye hemorrhage NOS | 1 | (8) | 0 | 0 | 0 | 1 | (2) |
| Eye injury NOS | 1 | (8) | 0 | 0 | 0 | 1 | (2) |
| Feces Discolored | 1 | (8) | 0 | 0 | 0 | 1 | (2) |

| | | | | | | | |
|---|---|---|---|---|---|---|---|
| Fibrosis NOS | 0 | 1 | (8) | 0 | 0 | 1 | (2) |
| Flatulence | 0 | 1 | (8) | 0 | 0 | 1 | (2) |

| | | | | | | | |
|---|---|---|---|---|---|---|---|
| Fungal infection | 0 | 0 | 1 | (6) | 0 | 1 | (2) |

| | | | | | | | |
|---|---|---|---|---|---|---|---|
| Gastroesophageal reflux disease | 1 | (8) | 0 | 0 | 0 | 1 | (2) |
| Hematoma NOS | 1 | (8) | 0 | 0 | 0 | 1 | (2) |

| | | | | | | | |
|---|---|---|---|---|---|---|---|
| Hematuria | 0 | 0 | 1 | (6) | 0 | 1 | (2) |
| Hemoptysis | 0 | 0 | 1 | (6) | 0 | 1 | (2) |
| Hemorrhage | 0 | 0 | 1 | (6) | 0 | 1 | (2) |

| | | | | | | | |
|---|---|---|---|---|---|---|---|
| Hyperkeratosis | 0 | 1 | (8) | 0 | 0 | 1 | (2) |
| Hypoesthesia | 0 | 1 | (8) | 0 | 0 | 1 | (2) |

| | | | | | | | | |
|---|---|---|---|---|---|---|---|---|
| Idiopathic thrombocytopenic | 1 | (8) | 0 | 0 | 1 | (25) | 1 | (2) |
| purpura | | | | | | | | |

| | | | | | | | |
|---|---|---|---|---|---|---|---|
| Joint dislocation | 0 | 0 | 1 | (6) | 0 | 1 | (2) |

| | | | | | | | |
|---|---|---|---|---|---|---|---|
| Joint swelling | 0 | 1 | (8) | 0 | 0 | 1 | (2) |

| | | | | | | | |
|---|---|---|---|---|---|---|---|
| Laryngitis | 0 | 0 | 1 | (6) | 0 | 1 | (2) |
| Lethargy | 0 | 0 | 1 | (6) | 0 | 1 | (2) |

| | | | | | | | |
|---|---|---|---|---|---|---|---|
| Lipodystrophy acquired | 0 | 1 | (8) | 0 | 0 | 1 | (2) |

| | | | | | | | |
|---|---|---|---|---|---|---|---|
| Loose stools | 0 | 0 | 1 | (6) | 0 | 1 | (2) |

| | | | | | | | |
|---|---|---|---|---|---|---|---|
| Menstruation irregular* | 0 | 1 | (11) | 0 | 0 | 1 | (3) |

| | | | | | | | |
|---|---|---|---|---|---|---|---|
| Migraine | 0 | 0 | 1 | (6) | 0 | 1 | (2) |

| | | | | | | | |
|---|---|---|---|---|---|---|---|
| Muscle spasms | 0 | 1 | (8) | 0 | 0 | 1 | (2) |
| Myalgia | 0 | 1 | (8) | 0 | 0 | 1 | (2) |

| | | | | | | | |
|---|---|---|---|---|---|---|---|
| Nail bed bleeding | 0 | 0 | 1 | (6) | 0 | 1 | (2) |

| | | | | | | | |
|---|---|---|---|---|---|---|---|
| Nasopharyngitis | 0 | 1 | (8) | 0 | 0 | 1 | (2) |

| | | | | | | | | |
|---|---|---|---|---|---|---|---|---|
| Neck pain | 1 | (8) | 0 | 0 | 1 | (25) | 1 | (2) |

| | | | | | | | |
|---|---|---|---|---|---|---|---|
| Oral mucosal petechiae | 0 | 1 | (8) | 0 | 0 | 1 | (2) |
| Osteopenia | 0 | 1 | (8) | 0 | 0 | 1 | (2) |

| | | | | | | | |
|---|---|---|---|---|---|---|---|
| Paraesthesia | 1 | (8) | 0 | 0 | 0 | 1 | (2) |

| | | | | | | | |
|---|---|---|---|---|---|---|---|
| Premenstrual syndrome* | 0 | 1 | (11) | 0 | 0 | 1 | (3) |

| | | | | | | | |
|---|---|---|---|---|---|---|---|
| Pruritus | 0 | 0 | 1 | (6) | 0 | 1 | (2) |

| | | | | | | | |
|---|---|---|---|---|---|---|---|
| Pyrexia | 0 | 1 | (8) | 0 | 0 | 1 | (2) |

| Rash | 0 | 0 | 1 | (6) | 0 | | 1 | (2) |
|---|---|---|---|---|---|---|---|---|
| Rash papular | 0 | 0 | 1 | (6) | 0 | | 1 | (2) |
| Rash scaly | 0 | 0 | 1 | (6) | 0 | | 1 | (2) |
| Rectal hemorrhage | 0 | 0 | 1 | (6) | 2 | (50) | 1 | (2) |

| | | | | | | | |
|---|---|---|---|---|---|---|---|
| Retinal hemorrhage | 0 | 1 | (8) | 0 | 0 | 1 | (2) |

| | | | | | | | |
|---|---|---|---|---|---|---|---|
| Rigors | 1 | (8) | 0 | 0 | 0 | 1 | (2) |

| | | | | | | | |
|---|---|---|---|---|---|---|---|
| Scoliosis | 0 | 1 | (8) | 0 | 0 | 1 | (2) |
| Sinus headache | 0 | 1 | (8) | 0 | 0 | 1 | (2) |

| | | | | | | | |
|---|---|---|---|---|---|---|---|
| Sinusitis | 0 | 0 | 1 | (6) | 0 | 1 | (2) |

| | | | | | | | |
|---|---|---|---|---|---|---|---|
| Sinusitis acute NOS | 1 | (8) | 0 | 0 | 0 | 1 | (2) |

| | | | | | | | |
|---|---|---|---|---|---|---|---|
| Skin laceration | 0 | 1 | (8) | 0 | 0 | 1 | (2) |

| | | | | | | | |
|---|---|---|---|---|---|---|---|
| Skin ulcer | 0 | 0 | 1 | (6) | 0 | 1 | (2) |
| Sleep disorder | 0 | 0 | 1 | (6) | 0 | 1 | (2) |

| | | | | | | | |
|---|---|---|---|---|---|---|---|
| Tachycardia NOS | 0 | 1 | (8) | 0 | 0 | 1 | (2) |

| | | | | | | | |
|---|---|---|---|---|---|---|---|
| Thermal burn | 0 | 0 | 1 | (6) | 0 | 1 | (2) |

| | | | | | | | |
|---|---|---|---|---|---|---|---|
| Tongue hemorrhage | 0 | 1 | (8) | 0 | 0 | 1 | (2) |

| | | | | | | | |
|---|---|---|---|---|---|---|---|
| Tremor | 0 | 0 | 1 | (6) | 0 | 1 | (2) |

| | | | | | | | |
|---|---|---|---|---|---|---|---|
| Upper respiratory tract | 1 | (8) | 0 | 0 | 0 | 1 | (2) |
| infection viral NOS | | | | | | | |

| | | | | | | | |
|---|---|---|---|---|---|---|---|
| Urinary tract infection | 0 | 0 | 1 | (6) | 0 | 1 | (2) |

| | | | | | | | |
|---|---|---|---|---|---|---|---|
| Varicose veins NOS | 0 | 1 | (8) | 0 | 0 | 1 | (2) |
| Vascular insufficiency | 0 | 1 | (8) | 0 | 0 | 1 | (2) |

| | | | | | | | |
|---|---|---|---|---|---|---|---|
| Vertigo | 1 | (8) | 0 | 0 | 0 | 1 | (2) |

| | | | | | | | |
|---|---|---|---|---|---|---|---|
| Vision blurred | 0 | 1 | (8) | 0 | 0 | 1 | (2) |

| | | | | | | | |
|---|---|---|---|---|---|---|---|
| Vomiting | 0 | 0 | 1 | (6) | 0 | 1 | (2) |

| | | | | | | | |
|---|---|---|---|---|---|---|---|
| Vomiting NOS | 1 | (8) | 0 | 0 | 0 | 1 | (2) |
| Wound NOS | 1 | (8) | 0 | 0 | 0 | 1 | (2) |

| | | | | | | |
|---|---|---|---|---|---|---|
| Asthma | 0 | 0 | 0 | 1 | (25) | 0 |
| Confusional state | 0 | 0 | 0 | 1 | (25) | 0 |
| Deep vein thrombosis | 0 | 0 | 0 | 1 | (25) | 0 |
| Eye hemorrhage | 0 | 0 | 0 | 1 | (25) | 0 |
| Flank pain | 0 | 0 | 0 | 1 | (25) | 0 |
| Hemorrhage intracranial | 0 | 0 | 0 | 1 | (25) | 0 |
| Injection site pain | 0 | 0 | 0 | 1 | (25) | 0 |
| Mouth hemorrhage | 0 | 0 | 0 | 1 | (25) | 0 |

NOS = not otherwise specified
*Proportions based on number of female (or male) patients

XI. The following example describes Study Part B.

### Study Population

Twenty-one patients were enrolled: Four patients randomized to placebo, 8 to each of the 1 μg/kg and 3 μg/kg cohorts, and 1 to the 6 μg/kg cohort. The placebo and the AMP2 molecule groups were comparable with respect to demographics and baseline platelet count (see Table II). Fifteen of the 21 patients were female and 14 were white. Median age was 49 years, median weight was 78 kg, and median platelet count at baseline was 16×10⁹/L. The median time since the diagnosis of ITP was 5.2 years. Seven patients (33%) were using corticosteroids at baseline, and 14 (67%) had undergone a splenectomy.

### Efficacy

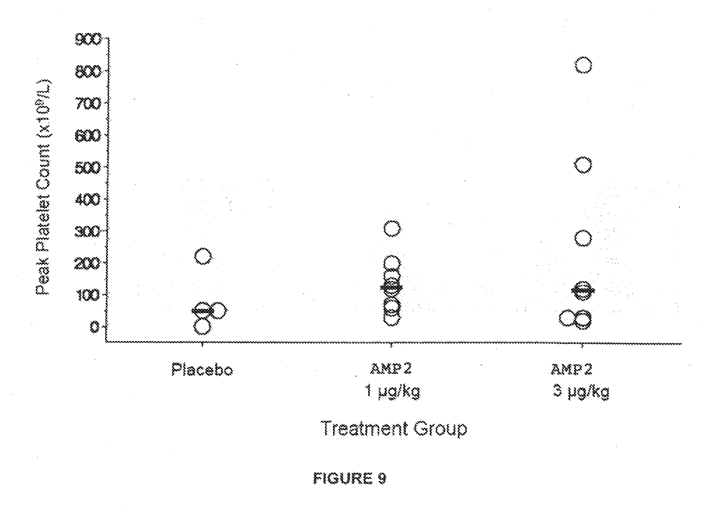

The only patient assigned to the 6 μg/kg dose had a platelet count increase to 520×10⁹/L on day 21. Because of the high count and the probability that the patient received the highest dose (later confirmed), the Data Review Committee closed the 6 μg/kg cohort to additional patients. Weekly doses of an AMP2 molecule at 1 and 3 μg/kg substantially increased the platelet count in most patients (FIG. 9). In the 1 μg/kg cohort, 7 of 8 patients (88%) achieved the target platelet response (doubling of baseline and platelet count of 50-450×10⁹/L). Five of eight patients (63%) in the 3 μg/kg cohort achieved (3 patients) or exceeded (2 patients) the target platelet response. Overall, 12 of 16 patients (75%) treated with an AMP2 molecule achieved (10 patients) or exceeded (2 patients) the target platelet response, 9 by the first assessment on day 8.

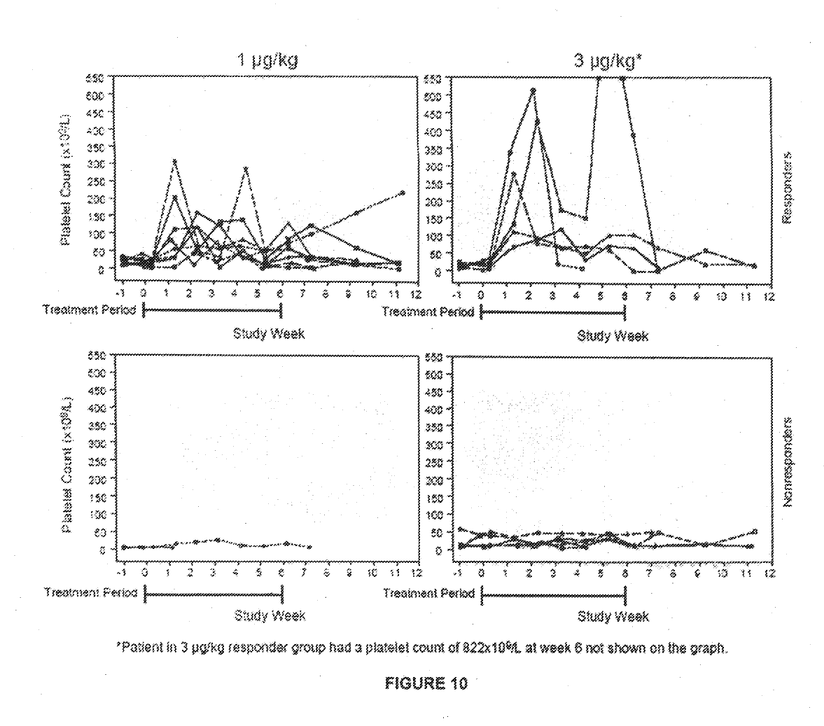

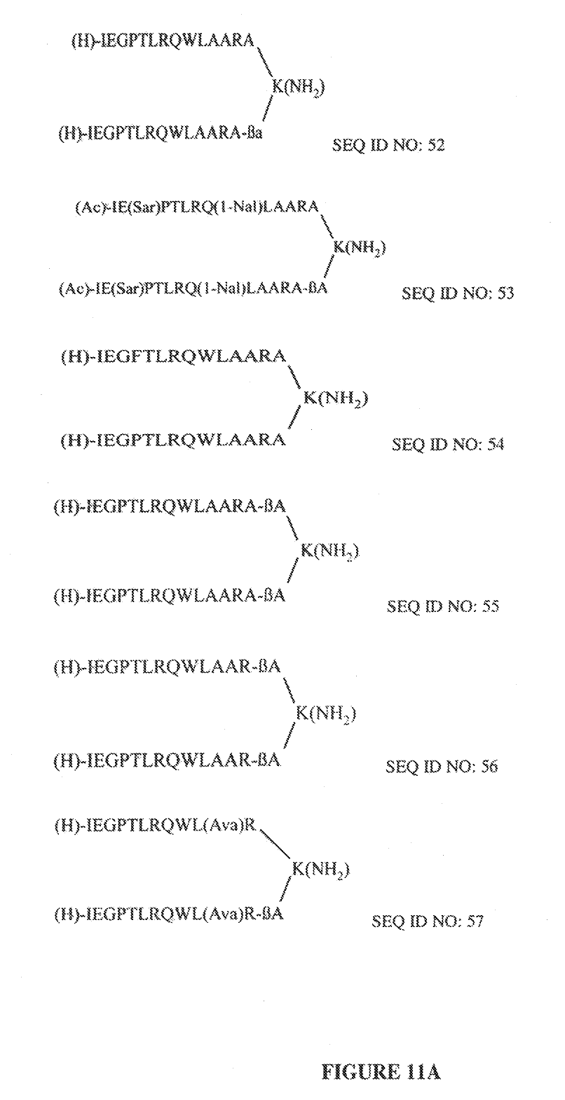

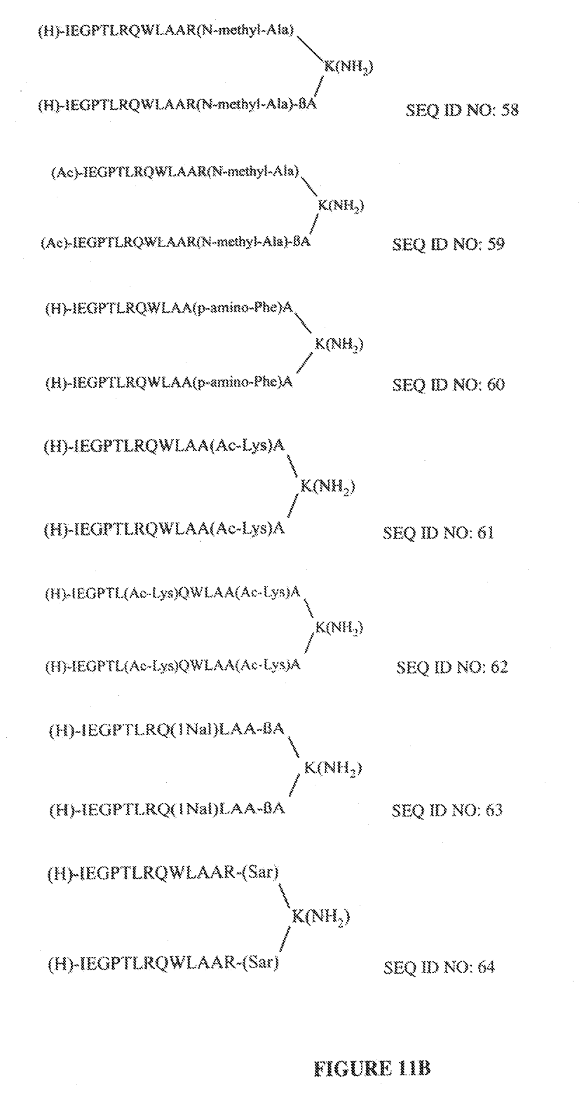

Five patients (63%) at each dose (1 and 3 μg/kg) achieved a peak platelet count of >100×10⁹/L. A total of 14 patients (88%) treated with an AMP2 molecule had a platelet count increase of =20×10⁹/L over their baseline count. The variability of individual platelet count responses over time for the 20 patients in the 1 μg/kg, 3 μg/kg, and placebo groups is shown in FIG. 10. The mean peak platelet count was 135×10⁹/L and 241×10⁹/L in the 1 μg/kg and 3 μg/kg cohorts, respectively, versus 81×10⁹/L for placebo. These counts represent mean increases to 8.5 fold, 17 fold, and 2.7 fold of baseline. One of the 4 placebo patients had a spontaneous remission; this patient had undergone a splenectomy 3.5 months before entering the study.

No statistically significant relationship was observed between peak platelet count and baseline TPO level.

### Safety

The adverse event data, as summarized in Table III, compare patients receiving an AMP2 molecule (n=17) and those receiving placebo (n=4). The AMP2 molecule was generally well tolerated at the doses tested in this study. The most frequently reported adverse events in the AMP2 molecule and placebo groups, respectively, were contusion/ecchymosis (59% and 75%), epistaxis (41% and 50%), mild to moderate headache (29% and 0%), and oral mucosal blistering (also 29% and 0%). All patients who reported oral mucosal blistering had experienced oral bleeding in the past including at entry into the study. Most bleeding events occurred during the post-treatment period or in non-responders.

Three patients (2 placebo and 1 AMP2 molecule) experienced serious adverse events. The 2 placebo-treated patients had a total of 3 serious adverse events: asthma in 1 patient and deep vein thrombosis following intracranial hemorrhage in a second patient. The patient treated with an AMP2 molecule (3 μg/kg) had vaginal bleeding in the setting of severe but transient thrombocytopenia 19 days after discontinuation of AMP2 molecule.

No patients in this study tested positive for anti-AMP2 or anti-TPO antibodies. With the exception of platelet counts, no clinically significant changes were observed in hematologic or serum chemistry laboratory values.

XII. The following example shows that an AMP2 molecule increased platelet counts in patients suffering from ITP in Both Studies A and B.

An AMP2 molecule, a novel thrombopoiesis-stimulating protein without sequence homology to TPO, was evaluated in a total of 41 patients with severe, refractory ITP, many of whom had been unresponsive to conventional treatments including splenectomy (n=32). No patients developed neutralizing antibodies to the AMP2 molecule. Except for headache and transient post-treatment thrombocytopenia, all toxicities appeared to be related to the underlying disease. Mild-to-moderate headache was reported in 36% of patients across both Study A and Study B. The headache occurred within 24 hours of treatment, was usually present for a few hours, responded to acetaminophen when required, and did not lead to discontinuation of treatment. The AMP2 molecule did not appear to affect the ongoing rate of platelet destruction, and most patients experienced a decrease in platelets back to their baseline values after discontinuation of treatment. However, in four patients, the platelet count fell below the patients' prior baseline. Some of these patients received rescue treatment with IVIG or corticosteroids. This transient post-treatment worsening of thrombocytopenia had no relationship to any clinical variable, such as the peak platelet count, and may reflect a temporary decrease in endogenous TPO due to its increased clearance by the pharmacologically expanded megakaryocyte mass (Kuter et al., Blood 100:3457-3469, 2002; Stoffel et al., Blood 87:567-573, 1996).

Patients completing this and other AMP2 molecule studies were eligible to enroll in an ongoing open-label extension study of weekly dosing, with dose adjustment based on platelet count. Preliminary data after 48 weeks of treatment have been reported elsewhere and have shown that treatment was generally effective and well tolerated (Bussel) et al., Blood 106:220a, 2005). However, two patients who had participated in the study described herein were observed to have a mild-to-moderate increase in bone marrow reticulin, but with no collagen fibrosis (Stepan et al., Blood 106:1240a, 2005). Both patients were asplenic, and both were receiving relatively high doses of an AMP2 molecule (>10 μg/kg) with minimal or no response. Increased reticulin is an anticipated and reversible effect of TPO stimulation, as has been seen in animal models (Ulich et al., Blood 87:5006-5015, 1996; Yanagida et al., Br. J. Haematol. 99:739-745, 1997) and in humans (Douglas et al., Am. J. Clin. Pathol. 117:844-850, 2002).

The AMP2 molecule was effective in substantially increasing the platelet count in ITP patients. Combining the results of effective doses in Part A (>1 μg/kg, n=12) and all doses in Part B (=1 μg/kg, n=17) of this study, the targeted platelet response (doubling of baseline and platelet count of 50-450×10⁹/L) was achieved by 14 of 29 patients (48%) treated. This protocol definition of efficacy underestimates the actual platelet responses. An additional six patients exceeded the targeted platelet count range. Thus, 20 of 29 patients (69%) met or exceeded the targeted platelet count. An even higher response rate may have been seen if all the patients received higher and/or multiple doses. No complications, including thrombotic events, were reported for patients with platelet counts above 450×10⁹/L.

In Part B of the study, considerable week-to-week fluctuation in platelet count response was observed despite constant dose administration. This variability suggests that individual dose adjustment may be required.

The mechanism by which an AMP2 molecule increases platelet counts may involve prevention of megakaryocyte apoptosis, as well as the stimulation of megakaryocyte progenitors and megakaryocyte maturation and endomitosis, as has been demonstrated with other thrombopoietic growth factors. In experiments in patients with thrombocytopenia related to HIV infection, PEG rHuMGDF decreased apoptosis of megakaryocytes and increased effective thrombopoiesis (Harker et al., Blood 92:707a, 1998).

An AMP2 molecule is a viable treatment strategy for patients with ITP because it appears to be both well tolerated and effective. Patients in this study were eligible to enroll in an open label study with an AMP2 molecule to assess long-term safety, efficacy, and durability of response. This ongoing study and other randomized trials will further define the role of an AMP2 molecule in the treatment of chronic ITP, including predictors of response, duration of response, thorough characterization of post-treatment thrombocytopenia, and bone marrow reticulin changes.

The foregoing description of a preferred embodiment of the invention has been presented for purposes of illustration and description, and is not intended to be exhaustive or to limit the invention to the precise form disclosed. The description was selected to best explain the principles of the invention and practical application of these principals to enable others skilled in the art to best utilize the invention in various embodiments and various modifications as are suited to the particular use contemplated. It is intended that the scope of the invention not be limited by the specification, but be defined by the claims set forth below.

The invention now being fully described, it will be apparent to one of ordinary skill in the art that many changes and modifications can be made thereto, without departing from the spirit and scope of the invention as set forth herein.

We claim:

1. A method of stimulating megakaryocyte or platelet production comprising administering a compound that binds to an mpl receptor in a dosage amount of about 3 μg/kg to about 10 μg/kg and comprising a structure selected from the group consisting of 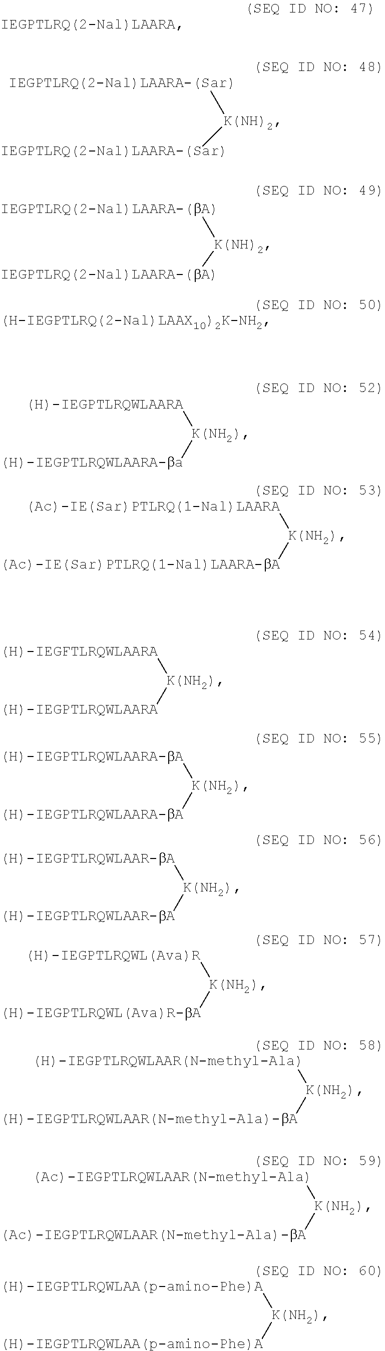 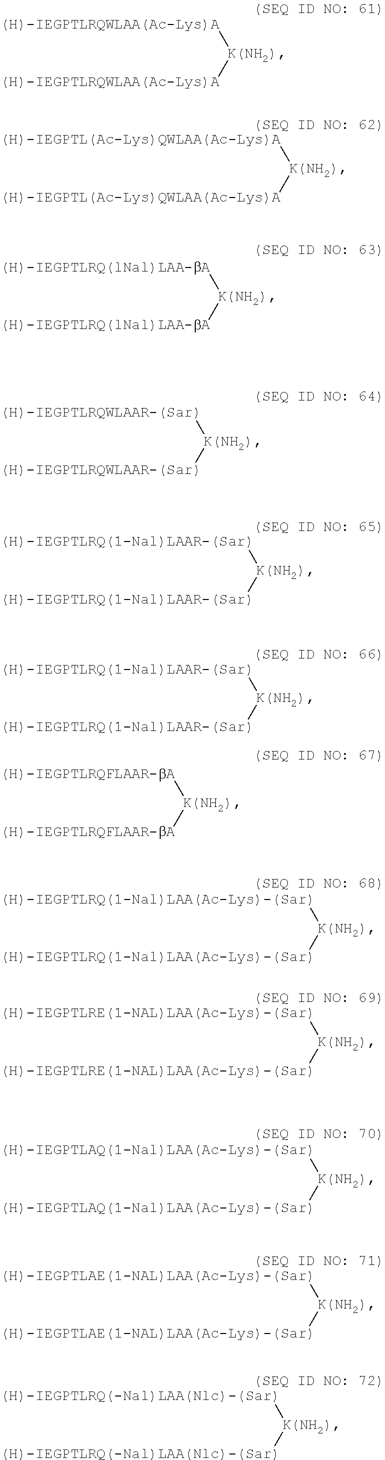 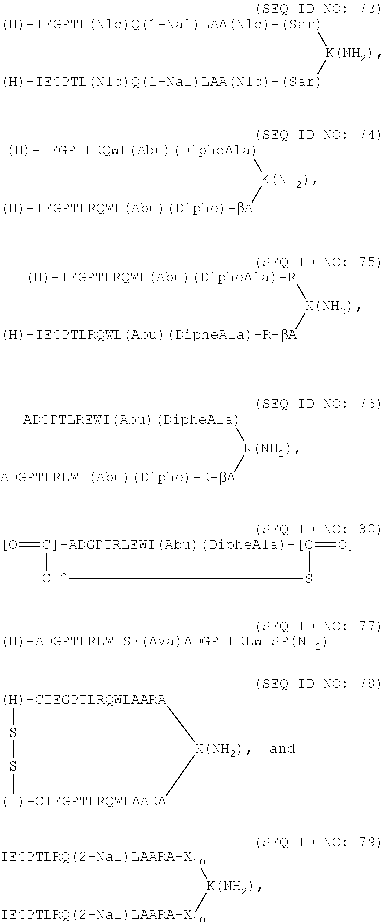
and pegylated forms thereof.

2. The method of any of claim 1, wherein said compound is formulated in amount to double megakaryocyte or platelet production over a baseline level.

3. The method of any of claim 1, said compound is formulated in amount to increase megakaryocyte or platelet production to a level of about 20×10⁹/L to about 2000×10⁹/L.

4. The method of claim 3, said compound is formulated in amount to increase megakaryocyte or platelet production to a level of about 50×10⁹/L to about 250×10⁹/L.

5. The method of claim 3, said compound is formulated in amount to increase megakaryocyte or platelet production to a level of about 300×10⁹/L to about 1000×10⁹/L.

6. The method of any of claim 1, wherein said disease state is a hepatic disease or condition associated with thrombocytopenia.

7. The method of claim 6, wherein said hepatic disease or condition is selected from the group consisting of alcoholic hepatitis, autoimmune hepatitis, drug-induced hepatitis, epidemic hepatitis, infectious hepatitis, long-incubation hepatitis, noninfectious hepatitis, serum hepatitis, short-incubation hepatitis, toxic hepatitis, transfusion hepatitis, viral hepatitis B (HBV), viral hepatitis C(HCV), viral hepatitis D (HDV), delta hepatitis, viral hepatitis E (HEV), viral hepatitis F (HFV), viral hepatitis G (HGV), liver disease, inflammation of the liver, and hepatic failure.

8. The method of any of claim 1, wherein the disease state is thrombocytopenia resulting from the treatment of AIDS.
